SEQUENCE LISTING (58 sequences)

SEQ ID NO: 1 - DNA, 8188 bp, Artificial Sequence
  Synthetic- Plasmid
gcggccgcgatctctcacctaccaaacaatgcccccctgcaaaaaataaattcatataaaaaacatacagataaccatctgcggtgataaattatctctggcggtgttgacataaataccactggcggtgatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaacccaatgaacaattggatgcgttgagctaaccggtactaatgaaccgtgaggcttaaccgagaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacgggaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagataactagtggagggcgcttaccactttgtgattcatgactggggtgaagtcgtaacaaggtaaccgtaggggaacctgcggttggatcacctccttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgagcttaagcttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggctttcctttccatcaaaaaaatattgatgaaatgagctgttgacaattaatcatcggctcgtataatgtgtggaattgtcacacaggaaacagaattcccggggatctgggggatcatcgatggttgtaaaagaattcggtggagcggtagttcagtcggttagaatacctgcctgtcacgcagggggtcgcgggttcgagtcccgtccgttccgccaccctaattaggggcgtagttcaattggtagagcaccggtctccaaaaccgggtgttgggagttcgagtctctccgcccctgccagaaatcatccttgtcgatgggagcagtaaaacctctacaggcttgtagctcaggtggttagagcgcacccctgataagggtgaggtcggtggttcaagtccactcaggcctaccaaatttgcacggcaaatttgaagaggttttaactacatgttatggggctatagctcagctgggagagcgcctgctttgcacgcaggaggtctgcggttcgatcccgcatagctccaccatctctgtagtgattaagagcgtgataagcaattttcgtgtccccttcgtctagaggcccaggacaccgccctttcacggcggtaacaggggttcgaatcccctaggggacgccactctaggaaatccgccataaaacaaaaggctcagtcggaagactgggccttttgttttacgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 2 - DNA, 9029 bp, Artificial Sequence
  Synthetic poRbs plasmid
gcggccgcgatctctcacctaccaaacaatgcccccctgcaaaaaataaattcatataaaaaacatacagataaccatctgcggtgataaattatctctggcggtgttgacataaataccactggcggtgatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccattccggggatccgtcgacctgcagttcgaagttcctattctctagaaagtataggaacttcagagcgcttttgaagctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctaataaggggatcttgaagttcctattccgaagttcctattctctagaaagtataggaacttcgaagcagctccagcctacaatcgatgcgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagc

SEQ ID NO: 3 - DNA, 9029 bp, Artificial Sequence; identical to SEQ ID NO: 2
  Synthetic- poGFP plasmid

SEQ ID NO: 4 - DNA, 7701 bp, Artificial Sequence
  Synthetic- poLuc plasmid
attcgcggccgcttctagagtccctatcagtgatagagattgacatccctatcagtgatagagatactgagcactactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggaatgcttagttttgcacattcagaaaaagacaactatatagatagtttatttttacatgcgtgtatgaacataccattaattgttccttctctagttgataattatcgaaaaataaatatagcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacaggtttacgcaagaaaatggtttgttatagtcgaataaactatatctgttattttttccaaccacagatctatggaagacgccaaaaacataaagaaaggcccggcgccattctatccgctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgccaaaaagttgcgcggaggagttgtgtttgtggacgaagtaccgaaaggtcttaccggaaaactcgacgcaagaaaaatcagagagatcctcataaaggccaagaagggcggaaagtccaaattgtaagtcgaccggctgctaacaaagcccgcggccgctgaagatcgatctcgacgagtgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtcaccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacactcgaggatctctttcctttccatcaaaaaaatattgatgaaatgagctgtggggcgttatttaggttttttcttctttcgaaaaaatctttctttatgaagttaaaagctatgtattcaatagcatattttgaatatggacatagaatagtgcttatcactattgcatatagcatcttatctgacacaaggaaataatacccttcgctgttttttgttataaggtatatatatataagtgtgcagtacaggccaaataaaatattttttatgtagtatcttaaatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagagctttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaaatccagatccttgacccgcatttgcaaaccctcactgatccgcatggggcccccgttccatacagaagctgggcgaagtaatcgcaacatattcgcggccgcttctagagtccctatcagtgatagagattgacatccctatcagtgatagagatactgagcactactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggaatgcttagttttgcacattcagaaaaagacaactatatagatagtttatttttacatgcgtgtatgaacataccattaattgttccttctctagttgataattatcgaaaaataaatatagccaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacaggtttacgcaagaaaatggtttgttatagtcgaataaactatatctgttattttttccaaccactgatccgcatggggcccccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacat

SEQ ID NO: 5 - DNA, 7701 bp, Artificial Sequence; identical to SEQ ID NO: 4
  Synthetic- poRFP/oGFP plasmid

SEQ ID NO: 6 - DNA, 1020 bp, Artificial Sequence
  Synthetic- GFP-TnaC template for in vitro translation
tcgatttttgtgatgctcgtcaggggggcggagcctatggaaacgaattcagatctcgatcccgcgaaattaatacgactcactatagggagaccacaacggtttccctctagaaataattttgtttaactttaagaaggagatatacatatgagcaaaggtgaagaactgtttaccggcgttgtgccgattctggtggaactggatggcgatgtgaacggtcacaaattcagcgtgcgtggtgaaggtgaaggcgatgccacgattggcaaactgacgctgaaatttatctgcaccaccggcaaactgccggtgccgtggccgacgctggtgaccaccctgacctatggcgttcagtgttttagtcgctatccggatcacatgaaacgtcacgatttctttaaatctgcaatgccggaaggctatgtgcaggaacgtacgattagctttaaagatgatggcaaatataaaacgcgcgccgttgtgaaatttgaaggcgataccctggtgaaccgcattgaactgaaaggcacggattttaaagaagatggcaatatcctgggccataaactggaatacaactttaatagccataatgtttatattacggcggataaacagaaaaatggcatcaaagcgaattttaccgttcgccataacgttgaagatggcagtgtgcagctggcagatcattatcagcagaataccccgattggtgatggtccggtgctgctgccggataatcattatctgagcacgcagaccgttctgtctaaagatccgaacgaaaaaggcacgcgggaccacatggttctgcacgaatatgtgaatgcggcaggtattacgtggagccataaatggttcaatattgacaacaaaattgtcgatcaccgcccttgatttgcccttctgtagccatcaccagagccaaaccgattagattcaatgtgatctatttgtttgctatatcttaattttgccttttgcaaaggtcatctctcgtttatttacttgttttag

SEQ ID NO: 7 - DNA, 900 bp, Artificial Sequence
  Synthetic- GFP-TnaC (W12R) template for in vitro translation
tcgatttttgtgatgctcgtcaggggggcggagcctatggaaacgaattcagatctcgatcccgcgaaattaatacgactcactatagggagaccacaacggtttccctctagaaataattttgtttaactttaagaaggagatatacatatgagcaaaggtgaagaactgtttaccggcgttgtgccgattctggtggaactggatggcgatgtgaacggtcacaaattcagcgtgcgtggtgaaggtgaaggcgatgccacgattggcaaactgacgctgaaatttatctgcaccaccggcaaactgccggtgccgtggccgacgctggtgaccaccctgacctatggcgttcagtgttttagtcgctatccggatcacatgaaacgtcacgatttctttaaatctgcaatgccggaaggctatgtgcaggaacgtacgattagctttaaagatgatggcaaatataaaacgcgcgccgttgtgaaatttgaaggcgataccctggtgaaccgcattgaactgaaaggcacggattttaaagaagatggcaatatcctgggccataaactggaatacaactttaatagccataatgtttatattacggcggataaacagaaaaatggcatcaaagcgaattttaccgttcgccataacgttgaagatggcagtgtgcagctggcagatcattatcagcagaataccccgattggtgatggtccggtgctgctgccggataatcattatctgagcacgcagaccgttctgtctaaagatccgaacgaaaaaggcacgcgggaccacatggttctgcacgaatatgtgaatgcggcaggtattacgtggagccataaacggttcaatattgacaacaaaattgtcgatcaccgcccttga

SEQ ID NO: 8 - DNA, 12 bp, Artificial Sequence
  Synthetic- Sequence in Figure 2b (left)
cgggaagacccc

SEQ ID NO: 9 - DNA, 13 bp, Artificial Sequence
  Synthetic- Sequence in Figure 2b (right)
cggaaagaccccc

SEQ ID NO: 10 - DNA, 12 bp, Artificial Sequence
  Synthetic- sequence in Figure 7b (left)
cgggaagacccc

SEQ ID NO: 11 - DNA, 13 bp, Artificial Sequence
  Synthetic- sequence in Figure 7b (right)
cggaaagaccccc

SEQ ID NO: 12 - DNA, 55 bp, Artificial Sequence
  Synthetic- primer NA1
aataggggttccgcgcacatttcccggcgcgtaaaacaaaaggcccagtcttccg

SEQ ID NO: 13 - DNA, 65 bp, Artificial Sequence
  Synthetic- Primer NA2
actcttcctgtcgtcatatctacaagccggctttcctttccatcaaaaaaatattgatgaaatga

SEQ ID NO: 14 - DNA, 25 bp, Artificial Sequence
  Synthetic- Primer NA3
tatacgagccgatgattaattgtca

SEQ ID NO: 15 - DNA, 18 bp, Artificial Sequence
  Synthetic- Primer NA4
gcctggcggcagtagcgc

SEQ ID NO: 16 - DNA, 56 bp, Artificial Sequence
  Synthetic- Primer NA5
aatcatggcaattctggaagaatagcgcggccgcgatctctcacctaccaaacaat

SEQ ID NO: 17 - DNA, 61 bp, Artificial Sequence
  Synthetic- Primer NA6
ctgcaggtcgacggatcccccggaatggcgcgccggcttgtagatatgacgacaggaagag

SEQ ID NO: 18 - DNA, 51 bp, Artificial Sequence
  Synthetic- Primer NA7
tcctgtcgtcatatctacaagccggcgcgccattccggggatccgtcgacc

SEQ ID NO: 19 - DNA, 50 bp, Artificial Sequence
  Synthetic- Primer NA8
gtctgaccactcggattatcccgcatcgattgtaggctggagctgcttcg

SEQ ID NO: 20 - DNA, 50 bp, Artificial Sequence; identical to SEQ ID NO: 19
  Synthetic- Primer NA9

SEQ ID NO: 21 - DNA, 55 bp, Artificial Sequence
  Synthetic- Primer NA10
attgtttggtaggtgagagatcgcggccgcgctatttcttccagaattcccatga

SEQ ID NO: 22 - DNA, 60 bp, Artificial Sequence
  Synthetic- Primer NA11
caagaaaatggtttgttatagtcgaataaactatatctgttattttttccaaccacagat

SEQ ID NO: 23 - DNA, 60 bp, Artificial Sequence
  Synthetic- Primer NA12
gaaagaagaaaaaacctaaataacgccccacagctcatttcatcaatatttttttgatgg

SEQ ID NO: 24 - DNA, 52 bp, Artificial Sequence
  Synthetic- Primer NA13
atagttggcgaagtaatcgcaacaataattcgcggccgcttctagagtccct

SEQ ID NO: 25 - DNA, 60 bp, Artificial Sequence
  Synthetic- Primer NA14
atctgtggttggaaaaaataacagatatagtttattcgactataacaaaccattttcttg

SEQ ID NO: 26 - DNA, 50 bp, Artificial Sequence
  Synthetic- Primer NA15
accctcactgatccgcatggggcccccgttccatacagaagctgggcgaa

SEQ ID NO: 27 - DNA, 50 bp, Artificial Sequence
  Synthetic- Primer NA16
agggactctagaagcggccgcgaatatgttgcgattacttcgccaactat

SEQ ID NO: 28 - DNA, 50 bp, Artificial Sequence
  Synthetic- Primer NA17
ttcgcccagcttctgtatggaacgggggccccatgcggatcagtgagggt

SEQ ID NO: 29 - DNA, 60 bp, Artificial Sequence
  Synthetic- Primer NA18
ccatcaaaaaaatattgatgaaatgagctgtggggcgttatttaggttttttcttctttc

SEQ ID NO: 30 - DNA, 30 bp, Artificial Sequence
  Synthetic- Primer NA19
aaactatcaggtcaagtctgcttttattat

SEQ ID NO: 31 - DNA, 29 bp, Artificial Sequence
  Synthetic- Primer NA20
cgagtaaataatattcacaatgtaccatt

SEQ ID NO: 32 - DNA, 52 bp, Artificial Sequence
  Synthetic- Primer NA21
ctgttattttttccaaccacagatctatggaagacgccaaaaacataaagaa

SEQ ID NO: 33 - DNA, 56 bp, Artificial Sequence
  Synthetic- Primer NA22
cggccgcgggctttgttagcagccggtcgacttacaatttggactttccgcccttc

SEQ ID NO: 34 - DNA, 76 bp, Artificial Sequence
  Synthetic- Primer NA23
tcgtcgagatcgatcttcagcggccgcgggctttgttagcagccggtcgactcaagggcggtgatcgacaattttg

SEQ ID NO: 35 - DNA, 54 bp, Artificial Sequence
  Synthetic- Primer NA24
tactgttattttttccaaccacagatctatgagcaaaggtgaagaactgtttac

SEQ ID NO: 36 - DNA, 25 bp, Artificial Sequence
  Synthetic- Primer NA25
caaaattgtcgatcaccgcccttga

SEQ ID NO: 37 - DNA, 20 bp, Artificial Sequence
  Synthetic- Primer NA26
acgttcaaatccgctcccgg

SEQ ID NO: 38 - DNA, 70 bp, Artificial Sequence
  Synthetic- Primer NA27
cagaacatattgactatccggtattacccggcatgacaggagtaaaaatggtgtaggctggagctgcttc

SEQ ID NO: 39 - DNA, 70 bp, Artificial Sequence
  Synthetic- Primer NA28
atgcgacccttgtgtatcaaacaagacgattaaaaatcttcgttagtttcatgggaattagccatggtcc

SEQ ID NO: 40 - DNA, 26 bp, Artificial Sequence
  Synthetic- Primer NA29
tcgatttttgtgatgctcgtcagggg

SEQ ID NO: 41 - DNA, 55 bp, Artificial Sequence
  Synthetic- Primer NA30
atcgacaattttgttgtcaatattgaaccatttatggctccacgtaatacctgcc

SEQ ID NO: 42 - DNA, 52 bp, Artificial Sequence
  Synthetic- Primer NA31
gacaattttgttgtcaatattgaaccgtttatggctccacgtaatacctgcc

SEQ ID NO: 43 - DNA, 60 bp, Artificial Sequence
  Synthetic- Primer NA32
gtgaatgcggcaggtattacgtggagccataaatggttcaatattgacaacaaaattgtc

SEQ ID NO: 44 - DNA, 60 bp, Artificial Sequence
  Synthetic- Primer NA33
gtgaatgcggcaggtattacgtggagccataaacggttcaatattgacaacaaaattgtc

SEQ ID NO: 45 - DNA, 30 bp, Artificial Sequence
  Synthetic- Primer NA34
ctaaaacaagtaaataaacgagagatgacc

SEQ ID NO: 46 - DNA, 50 bp, Artificial Sequence
  Synthetic- Primer NA35
cagcactgtgcaaacacgaaagtggacgtatacggtgtgacgcctgcccg

SEQ ID NO: 47 - DNA, 60 bp, Artificial Sequence
  Synthetic- Primer NA36
tgcaggtcgacggatccccggaatggcgcgtattcgagccggatgagtaattgtcaattt

SEQ ID NO: 48 - DNA, 17 bp, Artificial Sequence
  Synthetic- A2058G primer
gtaaggttcacggggtc

SEQ ID NO: 49 - DNA, 20 bp, Artificial Sequence
  Synthetic- A2451C primer
ctcttgggcggtatcagcct

SEQ ID NO: 50 - DNA, 19 bp, Artificial Sequence
  Synthetic- A2451C primer
cgtaccactttaaatggcg

SEQ ID NO: 51 - DNA, 20 bp, Artificial Sequence
  Synthetic- A2602U primer
cacggcagatagggaccgaa

SEQ ID NO: 52 - DNA, 25 bp, Artificial Sequence
  Synthetic- A2060C primer
aagctatagtaaaggttcacggggt

SEQ ID NO: 53 - DNA, 25 bp, Artificial Sequence
  Synthetic- A2062G primer
tgtcaagctatagtaaaggttcacg

SEQ ID NO: 54 - DNA, 25 bp, Artificial Sequence
  Synthetic- A2450U primer
tgaactcttgggcggtatcagcctg

SEQ ID NO: 55 - DNA, 25 bp, Artificial Sequence
  Synthetic- U2500G primer
ccccaggatgtgatgagccgacatg

SEQ ID NO: 56 - DNA, 20 bp, Artificial Sequence
  Synthetic- A2503G primer
ccaggatgtgatgagccgac

SEQ ID NO: 57 - DNA, 23 bp, Artificial Sequence
  Synthetic- C2611U primer
ttctccagcgcccacggcagata

SEQ ID NO: 58 - DNA, 23 bp, Artificial Sequence
  Synthetic- C2611G primer
ttctccagcgcccacggcagata

(19) United States
(12) Patent Application Publication
(10) Pub. No.: US 2023/0117150 A1
(43) Pub. Date: Apr. 20, 2023

(54) FULLY ORTHOGONAL SYSTEM FOR PROTEIN SYNTHISIS IN BACTERIAL CELLS

(71) Applicants: Northwestern University, Evanston, IL (US); THE BOARD OF TRUSTEES OF THE UNIVERSITY OF ILLINOIS, Urbana, IL (US)

(72) Inventors: Michael C. Jewett, Evanston, IL (US); Alexander S. Mankin, River Forest, IL (US); Nikolay Aleksashin, Chicago, IL (US)

(21) Appl. No.: 17/907,191

(22) PCT Filed: Mar. 24, 2021
(86) PCT No.: PCT/US2021/024006

§ 371 Date: Sep. 23, 2022

Related U.S. Application Data
(60) Provisional application No. 62/993,860, filed on Mar. 24, 2020.

(51) Int. Cl. C12P 21/02; C12N 9/10; C12N 9/14

(52) U.S. Cl. CPC C12P 21/02; C12N 9/104; C12Y 203/02012; C12N 9/14

(57) ABSTRACT

Disclosed are engineered polynucleotides, engineered ribosomes comprising the engineered polynucleotides, engineered cells and systems comprising the engineered polynucleotides and ribosomes, and methods of making and using the engineered polynucleotides, engineered ribosomes, engineered cells and systems. The engineered polynucleotides, engineered ribosomes, and engineered cells may be utilized to prepare sequence defined polymers and to select for mutant ribosomes that are capable of incorporating non-canonical amino acids into a polymer.

### CROSS-REFERENCE TO RELATED PATENT APPLICATIONS

This application claims the benefit of priority under 35 U.S.C. § 119(e) to U.S. Provisional Application No. 62/993,860, filed on Mar. 24, 2020, which content is incorporated herein by reference in its entirety.

### STATEMENT REGARDING FEDERALLY SPONSORED RESEARCH OR DEVELOPMENT

This invention was made with government support under MCB-1716766 and MCB-1615851 awarded by the National Science Foundation. The government has certain rights in the invention.

### FIELD

This invention pertains to engineered polynucleotides, engineered ribosomes comprising the engineered polynucleotides, engineered cells and systems comprising the engineered polynucleotides and ribosomes, and methods of making and using the engineered polynucleotides, engineered ribosomes, engineered cells and systems. The engineered polynucleotides, engineered ribosomes, and engineered cells may be utilized to prepare sequence defined polymers.

### BACKGROUND

The ribosome is a ribonucleoprotein machine responsible for protein synthesis. In all kingdoms of life it is composed of two subunits, each built on its own ribosomal RNA (rRNA) scaffold. The independent but coordinated functions of the subunits, including their ability to associate at initiation, rotate during elongation, and dissociate after protein release, are an established paradigm of protein synthesis. Furthermore, the bipartite nature of the ribosome is presumed essential for biogenesis since dedicated assembly factors keep immature ribosomal subunits apart and prevent them from translation initiation [Karbstein 2013]. Free exchange of the subunits limits the development of specialized orthogonal genetic systems that could be evolved for novel functions without interfering with native translation.

The ribosome is an extraordinary complex machine. This large particle, in which RNA is the main structural and functional component, is invariably comprised of two subunits that coordinate distinct but complementary functions: the small subunit decodes the mRNA, while the large subunit catalyzes peptide-bond formation and provides the exit tunnel for the polypeptide. The association of the subunits is tightly regulated throughout the cycle of translation. First, several assembly factors prevent the two subunits from associating during maturation of the ribonucleoproteins. Later on, the initiation of translation is also strictly controlled such that initiation factors, mRNA and fMet-tRNA^{fMet} sequentially join the small subunit to form a pre-initiation complex before recruiting the large subunit. During elongation, the subunits ratchet relative to each other with an angle of about 6 degrees. Upon termination, the newly synthesized protein is released from the ribosome and the subunits dissociate during an active process called ribosome recycling to prepare for additional rounds of translation. Thus, the requirement for programmed subunit association and dissociation at specific stages of translation likely explains why the ribosome has been maintained as two subunits during the course of evolution. Although initiation at the leaderless mRNAs was suggested to be carried out by the 70S ribosome with pre-associated subunits, no experimental evidence exists showing that the full cycle of protein synthesis could be accomplished by the ribosome with inseparable subunits.

The random exchange of ribosomal subunits between recurrent acts of protein biosynthesis presents an obstacle for making fully orthogonal ribosomes, a task with important implications for both fundamental science and bioengineering. Previously, it was possible to redirect a subpopulation of the small ribosomal subunits from translating indigenous mRNA to translation of a specific mRNA by placing an alternative Shine-Dalgarno (SD) sequence in a reporter mRNA and introducing the complementary changes in the anti-SD region in 16S rRNA [Hui 1987; Rackham 2005], which enabled selection of mutant 30S subunits with new decoding properties [Wang 2007]. However, because large subunits freely exchange between native and orthogonal small subunits, creating a fully orthogonal ribosome has been impossible thereby limiting the engineering of the 50S subunit, including the peptidyl transferase center (PTC) and the nascent peptide exit tunnel, for specialized new properties.

The engineering of a tethered ribosome, in which the subunits are linked to each other, could open new venues preparing orthogonal translation systems, evolving the ribosome for the incorporation of unnatural amino acids in synthetic biology, and molecularly characterizing dominant lethal mutations. Previously, we and others disclosed tethered ribosomes and methods of making and using tethered ribosomes. (See International Published Application WO 2015/184283, “Tethered Ribosomes and Methods of Making and Using Thereof,” and Orelle et al., “Protein synthesis by ribosomes with tethered subunits,” Nature, 6 Aug. 2015, Vol. 524, page 119). Here, we disclose further improvements to systems and methods that incorporate ribosomes with tethered subunits.

### SUMMARY

Disclosed herein are engineered polynucleotides, engineered ribosomes, and engineered cells and systems. The engineered polynucleotides, engineered ribosomes, and engineered cells and systems may be utilized in methods for preparing sequence defined polymers. In some embodiments, the engineered ribosomes comprise a small subunit, a large subunit, and a linking moiety comprising a polynucleotide sequence, wherein the linking moiety tethers the small subunit with the large subunit and wherein the engineered ribosome is capable of supporting translation of a sequence defined polymer.

In certain embodiments, the small subunit of the engineered ribosomes comprises rRNA and protein, the large subunit of the engineered ribosomes comprises rRNA and protein, and the linking moiety tethers the rRNA of the small subunit with the rRNA of the large subunit. In certain embodiments, the large subunit comprises a permuted variant of a 23S rRNA. In certain embodiments, the small subunit comprises a permuted variant of a 16S rRNA. As such, in certain embodiments, the engineered ribosomes comprise an engineered polynucleotide comprising a fusion of: (a) 16S rRNA, a permuted variant thereof, or fragments thereof; and (b) 23S rRNA, a permuted variant thereof, or fragments thereof.

The rRNA of the small subunit of the disclosed engineered ribosomes may comprise an anti-Shine-Dalgarno (anti-SD) sequence. In some embodiments, the anti-SD sequence of the rRNA of the small subunit of the engineered ribosomes corresponds or is identical to the native anti-SD sequence of an engineered host cell that comprises the engineered ribosome. In such embodiments, the anti-SD sequence of the rRNA of the small subunit of the engineered ribosomes exhibits reverse complementarity to the Shine-Delgarno (SD) sequence of the native mRNA's of the engineered host cell. In some embodiments of the disclosed engineered ribosomes, the rRNA of the small subunit of the disclosed engineered ribosomes is linked to the rRNA of the large submit via a linking moiety comprising a polynucleotide sequence, where the engineered ribosomes may be described as having tethered large subunits and small subunits and comprising a native anti-SD sequence of the engineered host cell that comprises the engineered ribosome which exhibits reverse complementarity to the SD sequence of native mRNA's of the engineered host cell. As such, the engineered ribosomes having tethered large subunits and small subunits may support translation using native mRNA's of the engineered host cell.

In other embodiments, the anti-SD sequence of the rRNA of the small subunit of the engineered ribosome is modified to include base substitutions relative to the anti-SD sequence of native mRNA's of an engineered host cell that comprises the engineered ribosome (or relative to the anti-SD sequence of the first engineered ribosome). In such embodiments, the engineered host cell may be engineered to comprise modified mRNA's having a modified anti-SD sequence that exhibits reverse complementarity to the modified anti-SD sequence of the rRNA of the small subunit of the engineered ribosome permitting translation of the modified mRNA's by the engineered ribosome having an rRNA with a modified anti-SD sequence.

The disclosed engineered ribosomes may be combined for use in engineered host cells. In some embodiments, the disclosed combination of ribosomes may include a first engineered ribosome and a second engineered ribosome. The first engineered ribosome may comprise: i) a small subunit comprising ribosomal RNA (rRNA) and protein, ii) a large subunit comprising ribosomal RNA (rRNA) and protein, and iii) a linking moiety; where the linking moiety comprises a polynucleotide sequence and tethers the rRNA of the small subunit with the rRNA of the large subunit. In some embodiments, the rRNA of the small subunit of the first engineered ribosome comprises an anti-SD sequence corresponding to the SD sequence of native mRNA's of the engineered host cell permitting translation of native mRNA's of the engineered host cell and preferably not permitting translation of mRNA's having a modified SD sequence (i.e., a modified SD sequence having one or more nucleotide substitutions relative to the anti-SD sequence of native ribosomes of the engineered host cell). The second engineered ribosome may comprise: i) a small subunit comprising rRNA and protein; and ii) a large subunit comprising rRNA and protein; where the second engineered ribosome lacks a linking moiety between the large subunit. In some embodiments, the rRNA of the small subunit of the second engineered ribosome comprises a modified anti-SD sequence having one or more nucleotide substitutions relative to the anti-SD sequence of native ribosomes of the engineered host cell (and/or relative to the anti-SD of the first engineered ribosome). The modified anti-SD sequence preferentially permits translation of mRNA templates having a complementary or cognate SD sequence that is different from the SD sequence of native cellular mRNAs and/or an anti-SD sequence that is different than the anti-SD sequence of the first engineered ribosome (i.e., permitting translation of mRNA's having a modified SD sequence that is complementary to the anti-SD of the rRNA of the small subunit of the second ribosome permitting translation of the mRNA's having a modified SD sequence by the second ribosome, and preferably not permitting translation of native mRNA's of the engineered host cell by the second engineered ribosome) and/or where the second engineered ribosome comprises one or more change-of-function mutations in the large subunit and/or small subunit relative to the native ribosomes of the engineered host cell (or relative to the first engineered ribosome) which change-of-function mutations are not present at the anti-SD sequence.

In certain embodiments where the large subunit and small subunit of the engineered ribosomes are tethered by a linking moiety, the linking moiety covalently bound a helix of the large subunit to a helix of the small subunit. In certain embodiments where the large subunit comprises a 23S rRNA (or a permuted variant of 23S rRNA) and the small subunit comprises a 16S rRNA (or a permuted variant of 16S rRNA), the linking moiety covalently bonds helix 10, helix 38, helix 42, helix 54, helix 58, helix 63, helix 78, or helix 101 of 23S rRNA (or a permuted variant of 23S rRNA) to a helix of 16S rRNA (or a permuted variant of 16S rRNA). In certain embodiments where the large subunit comprises a 23S rRNA (or a permuted variant of 23S rRNA) and the small subunit comprises a 16S rRNA (or a permuted variant of 16S rRNA), the linking moiety covalently bonds, the linking moiety covalently bonds helix 11, helix 26, helix 33, or helix 44 of 16S rRNA (or a permuted variant of 16S rRNA) to a helix of 23S rRNA (or a permuted variant of 23S rRNA).

In certain embodiments, the large subunit comprises a L1 polynucleotide domain, a L2 polynucleotide domain, and a C polynucleotide domain, wherein the L1 domain is followed, in order, by the C domain and the L2 domain, from 5′ to 3′. In certain embodiments, the polynucleotide consisting essentially of the L2 domain followed by the L1 domain, from 5′ to 3′, is substantially identical to 23S rRNA (e.g., 23S rRNA of E. coli). In certain embodiments, the polynucleotide consisting essentially of the L2 domain followed by the L1 domain, from 5′ to 3′, is at least 95% identical to a 23S rRNA. In certain embodiments, the C domain comprises a polynucleotide having a length ranging from 1-200 nucleotides. In certain embodiments, the C domain comprises a GAGA polynucleotide.

In certain embodiments, the small subunit comprises a S1 polynucleotide domain and a S2 polynucleotide domain, wherein the S1 domain is followed, in order, by the S2 domain, from 5′ to 3′. In certain embodiments, the polynucleotide consisting essentially of the S1 domain followed by the S2 domain, from 5′ to 3′, is substantially identical to a 16S rRNA (e.g., 16S rRNA of E. coli). In certain embodiments, the polynucleotide consisting essentially of the S1 domain followed by the S2 domain, from 5′ to 3′, is at least 95% identical to a 16S rRNA.

In certain embodiments, the linking moiety comprises a T1 polynucleotide domain and a T2 polynucleotide domain. In certain embodiments, the T1 domain links the S1 domain and the L1 domain and wherein the S1 domain is followed, in order, by the T1 domain and the L1 domain, from 5′ to 3′. In certain embodiments, the T1 domain comprises a polynucleotide having a length ranging from 5 to 200 nucleotides. In certain embodiments, the T1 domain comprises a polynucleotide having a length ranging from 7 to 40 nucleotides. In certain embodiments, the T1 domain comprises a polyadenine polynucleotide. In certain embodiments, the T1 domain comprises a polyadenine polynucleotide having a length of 7 to 12 adenine nucleotides. In certain embodiments, the T2 domain links the S2 domain and the L2 domain and wherein the L2 domain is followed, in order, by the T2 domain and the S2 domain, from 5′ to 3′. In certain embodiments, the T2 domain comprises a polynucleotide having a length ranging from 5 to 200 nucleotides. In certain embodiments, the T2 domain comprises a polynucleotide having a length ranging from 7 to 20 nucleotides. In certain embodiments, the T2 domain comprises a polyadenine polynucleotide. In certain embodiments, the T2 domain comprises a polyadenine polynucleotide having a length of 7 to 12 adenine nucleotides.

In certain embodiments, an engineered ribosome comprises the S1 domain followed, in order, by the T1 domain, the L1 domain, the C domain, the L2 domain, the T2 domain, and the S2 domain, from 5′ to 3′. In certain embodiments, the engineered ribosome comprises a polynucleotide consisting essentially of the S1 domain followed, in order, by the T1 domain, the L1 domain, the C domain, the L2 domain, the T2 domain, and the S2 domain, from 5′ to 3′.

In certain embodiments, the disclosed engineered ribosomes comprise a mutation relative to a wild-type host cell (e.g., relative to wild-type E. coli). In certain embodiments, the mutation is a change-of-function mutation. In certain embodiments, the change-of-function mutation is a gain-of-function mutation. In certain embodiments, the gain-of-function mutation is present in a peptidyl transferase center of the large subunit of the engineered ribosomes. In certain embodiments, the gain-of-function mutation is present in an A-site of the peptidyl transferase center of the large subunit of the engineered ribosomes. In certain embodiments, the gain-of-function mutation is present in the exit tunnel of the large subunit of the engineered ribosomes. In certain embodiments, the engineered ribosome comprise an antibiotic resistance mutation present in the large subunit and/or small subunit of the engineered ribosomes.

Also disclosed herein are polynucleotides, the polynucleotides encoding the rRNA of the engineered ribosomes. In certain embodiments, the polynucleotide is a vector. In certain embodiments, the polynucleotide further comprises a gene to be expressed by the engineered ribosome. In certain embodiments, the gene is a reporter gene. In certain embodiments, the reporter gene is a green fluorescent protein gene. In certain embodiments, the engineered ribosome comprises a modified anti-SD sequence and the gene comprises a complementary modified SD sequence corresponding to the anti-SD sequence of the engineered ribosomes. In certain embodiments, the gene comprises a codon and the codon encodes for an unnatural amino acid. In some embodiments, the ribosome comprising the modified anti-SD sequence is an untethered ribosome.

Also disclosed herein are methods for preparing an engineered ribosome, the method comprising expressing a polynucleotide encoding the rRNA of the engineered ribosome, for example, in an engineered host cell such as E. coli. In certain embodiments, the method further comprises preparing the engineered ribosome in a host cell, expressing a selectable marker, and selecting an engineered ribosome that expresses the selectable marker in the engineered host cell. In some embodiments, the selected engineered ribosome will include one or more mutations relative to the engineered ribosome that was expressed in the engineered host cell (and/or relative to the native ribosomes of the engineered host cell). In certain embodiments, the selection step comprises a negative selection step, a positive selection step, or both a negative and a positive selection step.

Also disclosed herein are engineered cells. The engineered cells are host cells, such as E coli cells, comprising (i) a polynucleotide encoding the rRNA of the engineered ribosome, (ii) the engineered ribosome, or both (i) and (ii). In some embodiments, the engineered host cells comprise a first engineered ribosome having a large subunit and a small subunit which are tethered, where the small subunit comprises rRNA having an anti-SD sequence corresponding to the SD sequence of native mRNA's of the engineered host cell. In some embodiments, the engineered host cells further comprise a second engineered ribosome having a large subunit and a small subunit which are not tethered, where the small subunit comprises rRNA having an anti-SD sequence which is modified relative to the SD sequence of native mRNA's of the engineered host cell and permits translation of mRNA's having a modified SD sequence corresponding to the modified anti-SD sequence of the rRNA of the small subunit of the second ribosome.

In some embodiments, the engineered cells comprise a first protein translation mechanism and a second protein translation mechanism. The first protein translation mechanism may comprise a first engineered ribosome, wherein the first engineered ribosome includes a linking moiety to tether the first and the second subunits. The second translation mechanism may comprise a second engineered ribosome, wherein the second engineered ribosome lacks a linking moiety between the large subunit and the small subunit. In some embodiments, the second engineered ribosome comprises a modified anti-SD sequence relative to the anti-SD sequence of native ribosomes which is complementary to the SD sequence of native mRNA's (and/or relative to the anti-SD sequence of the first engineered ribosome) and/or a change-of-function mutation other than at the anti-SD sequence relative to the native ribosomes of the engineered cells (and/or relative to the first engineered ribosome).

Also disclosed herein are methods for preparing a sequence-defined polymer, the methods comprising (a) providing an engineered ribosome or an engineered cell comprising one or more engineered ribosomes, and (b) providing an mRNA or DNA template encoding the sequence-defined polymer, and preparing the sequence-defined polymer using the one or more engineered ribosomes, the engineered cell comprising the one or more engineered ribosomes, and the mRNA or DNA template encoding the sequence-defined polymer. The sequence-defined polymer may be prepared in vitro and/or in vivo.

In certain embodiments, the sequence-defined polymer is prepared in vitro, and the methods further comprise providing (c) a ribosome-depleted cellular extract or purified translation system and using the ribosome-depleted cellular extract or purified translation system to preparing the sequence-defined polymer. In certain embodiments, the ribosome-depleted cellular extract comprises an S150 extract prepared from mid- to late-exponential growth phase cell cultures or cultures having an OD₆₀₀ of at least about 2.0, 2.5, or 3.0 at time of harvest.

In certain embodiments, the sequence defined polymer is prepared in vivo. The sequence defined polymer may be prepared in an engineered cell comprising a first and second translation system comprising engineered ribosomes, wherein the first translation system comprises tethered ribosomes having a wild-type anti-SD sequence (i.e., the native anti-SD sequence of ribosomes of the engineered host cell which is complementary to the SD sequence of native mRNA's of the engineered host cell), and wherein the second translation system comprises untethered ribosomes having (a) a modified anti-SD sequence (e.g., relative to the native anti-SD sequence of ribosomes of the engineered host cell or relative to the anti-SD of the tethered ribosomes of the first translation system), which is not complementary to the SD sequence of native mRNA's of the host cell) and/or (b) a change-of-function mutation other than at the anti-SD sequence, which mutation is relative to the native ribosomes of the engineered host cell or relative to the tethered ribosome of the first translation system. In certain embodiments, the mRNA or DNA encoding the sequence-defined polymer comprises a modified SD sequence and the untethered, engineered ribosome of the second translation system comprises a modified anti-SD sequence complementary to the modified SD sequence of the mRNA or DNA encoding the sequence-defined polymer permitting translation of the mRNA encoding the sequence-defined polymer permitting translation by the second translation system (and preferably permitting translation by the first translation system).

In certain embodiments, the sequence-defined polymer comprises an amino acid. In certain embodiments, the amino acid is a natural amino acid. In certain embodiments, the amino acid is an unnatural or non-canonical amino acid and the untethered, engineered ribosomes of the second translation system comprise one or more mutations relative to the native ribosomes (or relative to the tethered, engineered ribosomes of the first translation system), which permit incorporation of the unnatural or non-canonical amino acid into the sequence-defined polymer.

### BRIEF DESCRIPTION OF THE FIGURES

FIG. 1. The OSYRIS set-up. a) Organization of rRNA genes and structure of the dissociable 70S ribosome (left) and Ribo-T (right). The small and large subunits of Ribo-T are covalently linked by two RNA tethers connecting circularly-permutated 23S rRNA to the loop of helix 44 in 16S rRNA^{13,16}. b) In the original Ribo-T-based orthogonal translation system¹³, wt dissociable ribosomes translate the cellular proteome while the orthogonal Ribo-T (oRibo-T) is committed to the translation of the orthogonal reporter mRNA. c) In the OSYRIS cells (Example 1), the proteome is synthesized by Ribo-T whereas the dissociable ribosomes function as a specialized orthogonal translation system. The tethered nature of Ribo-T confines both subunits of the dissociable ribosome (the 30S subunit with altered ASD and the 50S subunit) to the translation of only the orthogonal mRNA.

FIG. 2. Performance of the dissociable orthogonal ribosome in the OSYRIS cells. a) Agarose gel electrophoresis analysis of the large rRNA species in the OSYRIS cells in comparison with wild type E. coli (wt), containing only dissociable 70S ribosomes, and with Ribo-T cells (Ribo-T) which only carry tethered ribosomes. b) Primer extension analysis of the ribosome content in the OSYRIS cells. Top: Ribo-T and the dissociable ribosome can be distinguished because of the A2058G mutation present in the Ribo-T rRNA. Middle: the principle of the primer extension analysis. In the presence of ddCTP, reverse transcriptase extends the primer by 4 nt on the 23S rRNA template (with A2058) but only by 3 nucleotides on the Ribo-T rRNA template (with G2058). Bottom: gel electrophoresis analysis of the primer extension products generated on the rRNA extracted from wt, Ribo-T, or OSYRIS cells. c) Expression of the orthogonal GFP reporter in OSYRIS cells carrying dissociable ribosomes with wt 30S subunit (wt Rbs) or orthogonal 30S subunit with altered ASD in the 16S rRNA (oRbs). Transcription of the reporter genes is induced by varying concentrations of the inducer, homoserine lactone¹⁹. The autofluorescence values of cells lacking the reporter gene were subtracted from all the values. The inset shows the UV light picture of the agar plate onto which the indicated cells were spotted and grown. d) Comparison of the expression of the o-gfp reporter in OSYRIS cells (dark grey bars) with that in BL21 cells transformed with o-pAM552 expressing wt ribosomes, or poRibo-T (light grey bars) (see Extended Data FIG. 1). The medium-copy number plasmids used to introduce o-ribosomes or oRibo-T into BL21 cells are based on the pBR322 replication origin (322); the low-copy number plasmid expressing o-ribosomes in OSYRIS is bases on pSC101 replication origin (101). Error bars show the s.d. for n=3 replicates. *** indicates p<0.0005 by Student's t-test.

FIG. 3. The orthogonality of the small and large subunits of the dissociable o-ribosome in the OSYRIS cells. a) Sensitivity of the expression of the orthogonal GFP reporter in the OSYRIS cells to erythromycin (left, dark grey bars) demonstrates that its translation is carried out primarily by the dissociable o-ribosome but not by the Ery^{R} Ribo-T or by a Ribo-T/30S hybrid (cartoon on the right). Consistently, translation of wt gfp gene, driven by Ery^{R} Ribo-T, is not inhibited by the antibiotic (light gray bars). Error bars show the s.d. for n=3 replicates. b) Top: OSYRIS cells transformed with a poRbs plasmid where the 23S rRNA gene contains the lethal mutation A2602U are able to form colonies, revealing that the large subunit of the o-ribosome does not participate in the translation of the cellular proteome. The dominant lethal nature of the A2602U mutation in a non-orthogonal translation system is demonstrated by the lack of colonies when OSYRIS cells are transformed with the same plasmid but with unaltered (wt) ASD in the 16S rRNA gene (pRbs) (see also FIG. 12b,c). Bottom: primer extension analysis showing that the OSYRIS cells stably maintain the large ribosomal subunits with 23S rRNA mutations that would be dominantly lethal in wt E. coli cells. cDNA bands generated by extending the primers annealed proximal to the relevant mutation site on the mutant 23S rRNA (upper arrows) or unmutated Ribo-T rRNA (lower arrows) are indicated. Co-existence of Ribo-T (with G2058) with dissociable ribosomes with lethal 23S rRNA mutations (but wt adenine at position 2058) was further confirmed by primer extension analysis around the 2058 rRNA residue (FIG. 12d). Right: cartoon illustrating the conclusions from these experiments which argue that the dissociable 50S subunits are largely isolated from the translation of the cellular proteome whose expression relies on Ribo-T.

FIG. 4. Selecting gain-of-function mutations from the PTC mutant library in the OSYRIS cells. a) Appending the TnaC-coding sequence to the end of gfp is expected to reduce the reporter expression due to the inhibitory action of TnaC on termination when translation occurs at high concentrations of L-tryptophan²⁸. The presence of the W12R mutation in TnaC is known to partially alleviate the termination problem²⁸ and should lead to a higher level of reporter expression. b) Expression of the GFP-TnaC fusion in the OSYRIS cells is inhibited by 94% in the presence of the L-tryptophan analog 1-methyl tryptophan (1m-Trp), while the expression of the GFP-TnaC(W12R) mutant decreases only by 48%. Error bars show the s.d. for n=3 replicates. ** indicates p<0.005 by Student's t-test. c) The location of the 23S rRNA nucleotides (arrow) whose mutations are present in the PTC mutant library shown on the cross-cut of the 50S ribosomal subunit. The P- and A-site tRNAs are shown. d) The 23S rRNA residues whose mutations comprise the PTC library. Left and middle: In the PTC library all the 23S rRNA residues within the 10 Å radius (the inner shell) and a large fraction of those within the 25 Å radius (second shell) of the PTC active site were mutated. The aminoacylated acceptor ends of the P- and A-site tRNAs are shown in pink and green, respectively. Right: positions of the mutated nucleotides shown in the secondary structure of the 23S rRNA domain V central loop. The relevant 23S rRNA hairpins are indicated. e) Translational activity and stalling bypass score of the PTC library mutants expressing the orthogonal GFP-TnaC reporter in the OSYRIS cells. The dots corresponding to the mutants exhibiting efficient termination of the TnaC peptide (increased bypass score) while maintaining high efficiency of translation (>60% of the wt control) are boxed and darker. The dotted line indicates the background level of expression of the orthogonal GFP-TnaC(W12R) mutant in the Ribo-T cells lacking the orthogonal ribosome. The black dot (arrow) shows the translation of the reporter by o-ribosomes that contains wt 23S rRNA. f) Testing isolated ribosomes with specific gain-of-function mutations identified in OSYRIS cells in a cell-free translation system. For in vitro testing, ribosomal 50S subunits with lethal mutations (U2500G, A2060C, A2450U) were isolated from OSYRIS cells and combined with wt 30S subunits. The ribosomes with non-lethal mutations were isolated from SQ171 cells. Toeprinting assay (FIG. 16c) was used to assess the extent of translation arrest at the stop codon of the tnaC gene due to inefficient release of TnaC. Error bars represent the standard deviation from three independent experiments. Statistical significance of the difference from wt values is indicated by * (p<0.05), ** (p<0.005), or *** (p<0.0005) determined by Student's t-test. g) The placement of the 23S rRNA residues (blue) whose mutations resulted in gain-of-function (dark dots in panel E) relative to the TnaC-tRNA (green) and RF2 (orange) in the structure of the TnaC-stalled ribosome³⁰.

FIG. 5 Key plasmids of the OSYRIS. a) The map of the pRibo-Tt plasmid. The pRibo-T genes encoding the 16S-23S rRNA hybrid and 5S rRNA are expressed under the control of the lambda P_{L} promoter. In the 16S-23S rRNA hybrid, the circularly permutated 23S rRNA opened at the loop of helix 101, is inserted into the loop of helix 44 of the 16S rRNA by way of two RNA tethers whose sequence in Ribo-T v.2.0 was redesigned relative to the original Ribo-T version^{3,4}. The 23S rRNA segment carries the A2058G mutation rendering Ribo-T erythromycin-resistant. The cluster of the tRNA genes, which are missing in the host cells due to the deletion of chromosomal rRNA operons¹⁰ is under control of the P_{tac} promoter. The plasmid carries the pBR322 origin of replication and an ampicillin resistance gene. b) The poRBS plasmid, derived from pAM552⁴, carries the E. coli rrnB operon with an altered ASD sequence GUGGUU in the 16S rRNA gene³. The plasmid carries the pSC101 origin of replication and a kanamycin resistance gene. The control plasmid pRbs (not shown) is identical to poRbs except that it contains wt ASD in the 16S rRNA genes. c) The reporter plasmids poGFP carry either the gene of the superfolder green fluorescent protein (sf-gfp) (poGFP) or the same gene and also the gene of the red fluorescent protein (poRFP/oGFP). The coding sequences of the reporter is preceded by the altered (orthogonal) SD sequence, AACCAC³ that is complementary to the ASD sequence in 16S rRNA encoded in the poRBS plasmid shown in panel b. Transcription of the orthogonal gfp gene in poGFP is controlled by the inducible P_{Lux} promoter regulated by binding of N-(β-ketocaproyl)-L-homoserine lactone (HSL) to the LuxR repressor. Two copies of the luxR gene are present in the plasmid. The poGFP plasmid pA15 origin of replication and Spc-resistance gene. d) The reporter plasmid poRFP/oGFP carries the genes for the green (sfGFP) and red (RFP) fluorescent proteins under control of the Pₗₚₚ₅ and P_{T5} promoters, respectively. Both genes are preceded by orthogonal SD sequence AACCAC. The plasmid has pA15 origin of replication and Spc-resistance gene. e) poLuc plasmid is similar to the poGFP plasmid (panel c), but the sf-gfp gene was replaced with the luc gene encoding firefly luciferase. The luc gene is preceded by an orthogonal SD sequence AACCAC. The fully annotated sequences of the plasmids shown in this figure can be found in Appendix I.

FIG. 6. The OSYRIS assembly in the E. coli cells. a, The plasmid composition of the OSYRIS cells. Ribo-T, that translates the cellular proteome, is expressed from the pRibo-Tt plasmid. The mRNA, transcribed from the orthogonal reporter gene on the poGFP (or poRFP/oGFP) plasmid, is translated by the o-ribosome whose rRNA is encoded in the poRbs plasmid. b, Sequential steps for the construction of the OSYRIS cells. The genome of the cells was completely sequenced after the assembly step III (see panel c). In the next two steps, cells were subsequently transformed with the reporter plasmids (poGFP in the illustrated example) and then with poRbs (or by the plasmids of the PTC mutant library described in FIG. 4). Antibiotics resistance of cells generated at every step is indicated. c, The genome of the OSYRIS cells. The starting SQ171 FG strain was derived from Escherichia coli MG1655 cells²⁶. Five spontaneous mutations (arrows) were acquired during OSYRIS cells assembly and propagation; the precise positions of the mutations and functions of the affected genes are listed in the table at FIG. 20. Numbers outside of the circle indicate genome nucleotide numbering. d, Gel electrophoresis analysis of the plasmid content of the cells from the different steps of OSYRIS assembly (shown in panel b). Plasmid preparations were digested with a mixture of KpnI, BamHI and HindIII restriction enzymes. Restriction digest of the individual plasmids is shown for reference.

FIG. 7. oRbs are stably expressed in the OSYRIS cells. a, Agarose gel-electrophoresis analysis of total RNA maintained in OSYRIS cells after dilution from the overnight culture. Two independent colonies (A and B) of OSYRIS cells with poGFP plasmid were grown overnight and diluted each 1:50 into two tubes with LB medium supplemented with 50 μg/ml Amp, 25 μg/ml Kan and 15 μg/ml Spc. Total RNA was isolated after indicated time intervals. Two technical replicates for each culture were processed independently and run in separate lanes of the gel. b, Primer extension analysis of the representation of oRbs (which has wt A2058) relative to Ribo-T (which carries the A2058G mutation) in the OSYRIS cells over time. The principle of primer extension analysis is illustrated above the sequencing gel. Total RNA prepared from OSYRIS cells (see panel a) was used as a template for primer extension. RNA samples prepared from wt E. coli cells (‘A2058’) and from cells expressing only Ribo-T (‘A2058G’) were used as controls. Lanes marked ‘Pr’ contain [³²P]-labeled DNA primer. c, Quantitation of the relative intensity of the Ribo-T and oRbs-specific bands was used to assess the relative representation of two ribosome species.

FIG. 8. Efficient translation of the orthogonal reporters in the OSYRIS cells. a, Growth curves (top) of the OSYRIS cells containing either o-ribosomes (solid lines) or wt ribosomes (dashed lines) and expression of the orthogonal GFP reporter therein (right). b, Growth curves (left) and expression of the orthogonal GFP (middle) and RFP reporters (right) in OSYRIS cells expressing o-ribosomes (solid lines) or wt ribosomes (dashed lines). The highest fluorescence reading (relative fluorescence units) in each experiment was taken as 100%. c, The expression of the orthogonal luciferase reporter in the OSYRIS cells containing either o-ribosomes (dark grey bars) or wt ribosomes (light grey bars). The highest luminescence reading (relative luminescence units, RLU) was taken as 100%. Error bars show the s.d. for n=3 replicates. *** indicates p<0.0005, n.s. indicates no statistical significance by Student's t-test.

FIG. 9. Expression of the orthogonal gfp reporter in OSYRIS cells and in E. coli BL21. (a) Growth curves, (b) GFP fluorescence, and (c) GFP fluorescence normalized by cell density in OSYRIS cells and BL21 cells grown in 96-well plates. Both types of cells express either wt ribosomes (dashed lines) or o-ribosomes (solid lines). Notice that the normalized orthogonal GFP fluorescence (or oGFP fluorescence per cell) is higher in the OSYRIS cells than in the BL21 cells. The data represent the results of three independent biological replicates; error bars indicate s.d. In (b), the highest fluorescence reading (relative fluorescence units) (for BL21 cells) was taken as 100%. In (c), the highest normalized fluorescence reading (relative fluorescence units over A₆₀₀) (for OSYRIS cells) was taken as 100%.

FIG. 10. oRbs outperforms oRibo-T in expression of orthogonal luciferase reporter. Expression of o-luc in BL21 or in OSYRIS cells driven by dissociable oRbs or oRibo-T. BL21 cells with the reporter plasmids poLuc were transformed with the medium copy number (pBR322 ori) plasmids o-pAM552 or with poRibo-T expressing oRbs or oRibo-T, respectively. OSYRIS cells express oRbs from a low copy number plasmid poRbs. Control cells were transformed with the same plasmids but carrying rRNA with wt ASD. The relative reporter expression was recorded as described in Experimental Procedures. Error bars show the s.d. for n=3 replicates. *** indicates p<0.0005 and n.s. indicates no statistical significance by Student's t-test.

FIG. 11. Resistance of the OSYRIS cells to erythromycin (Ery) illustrates the functional isolation of the orthogonal dissociable ribosome. a, Ribosome composition of the OSYRIS cells expressing wt (top two cells and left side of bottom cell) or orthogonal (right side of bottom cell) ribosomes. Tethered ribosomes carry the A2058G mutation rendering them resistant to Ery, whereas dissociable ribosomes are sensitive to Ery. b, Optical density of the OSYRIS cells cultures expressing wt or orthogonal ribosomes after 24 h growth in the 96-well plates in the presence of the indicated concentrations of Ery. Expressing wt dissociable Ery^{S} ribosomes alongside Ery^{R} Ribo-T renders cells sensitive to erythromycin (Ribo-T=+wt Rbs, third bar at each X axis point) indicating that if free 50S subunit is involved in translation (in this case due to its interaction with wt 30S subunit), protein synthesis and cell growth are inhibited. In contrast, cells expressing oRbs remain Ery^{R}, demonstrating that the 50S subunit of the orthogonal dissociable ribosome in the OSYRIS cells is functionally isolated and does not participate in the translation of the cellular proteome. For FIG. 11b, the for each X axis data value, the first bar is Ribo-T only; the second bar is Ribo-T+wt Rbs; the third bar is Ribo-T+oRbs.

FIG. 12. The viability of the OSYRIS cells expressing lethal mutations in the rRNA of the 50S subunit of the orthogonal ribosome demonstrates functional isolation of the two orthogonal translation systems. a, Locations of 23S rRNA nucleotides G2553, A2602, A2451 (orange) in the PTC active site (PDB 1VY4)²². Mutations of these nucleotides are dominantly lethal in wt E. coli cells²⁷. A-site tRNA is green, and P-site tRNA is blue. b, Transformation of the OSYRIS cells yields viable colonies when mutant 23S rRNA carrying lethal mutations is co-expressed with the orthogonal 16S rRNA (poRbs), but not when it is co-expressed with wt 16S rRNA (pRbs). c, Transformation of the POP2136 strain where the expression of the rRNAs operon from the pRbs and poRbs plasmids is repressed², yields no colonies. d, (Top) The ribosome composition in the OSYRIS cells expressing Ribo-T with the A2058G mutation and dissociable orthogonal ribosome (right) whose 50S subunit carries lethal mutations. (Bottom) Primer extension analysis of the rRNA region proximal to nucleotide 2058 showing stable maintenance in OSYRIS cells of the 50S subunits carrying lethal mutations (and no A2058G mutation) alongside with Ribo-T (carrying the A2058G mutation). Lanes 1-3: control primer extensions on preparations of the wt 23S rRNA (lane 1), 23S rRNA with the A2058G mutation (lane 2) or RNA extracted from the OSYRIS cells expressing only Ribo-T (lane 3). Lane 4, rRNA from the OSYRIS cells transformed with pRbs and expressing wt dissociable ribosome. Lanes 5-8, rRNA from the cells expressing orthogonal ribosome with no mutations in the 23S rRNA (lane 4) or with the indicated lethal mutations in the 23S rRNA. Numbers under the lanes of the gel indicate the content (%) of the 23S rRNA estimated as the ratio of the intensity of the cDNA band representing 23S rRNA (bottom two arrows) to the sum of intensities of the 23S rRNA- and Ribo-T-specific bands (bottom two arrows and top arrow, respectively). Shown is a representative gel of three independent biological replicates.

FIG. 13. TnaC-mediated inhibition of in vitro translation of the reporter protein. Translation of the GFP-TnaC or GFP-TnaC(W12R) reporters was carried out in the PURExpress cell-free system in the presence of low (50 μM) or high (5 mM) L-tryptophan concentration. The TnaC mutation W12R is known to diminish the TnaC-mediated inhibition of the protein release at the stop codon at high L-tryptophan concentration²⁸. The data represent the results of the three independent experiments, and the error bars indicate the experimental error. The sequences of the DNA templates can be found in Appendix I. The highest fluorescence reading (relative fluorescence units) in each experiment was taken as 100%.

FIG. 14. The translation activity of the PTC library mutants in OSYRIS cells. Translation activity of the individual mutants was estimated by comparing the expression of the o-GFP-TnaC (W12R) reporter (which shows partial stalling relieve) (see FIG. 4a-b) in the OSYRIS cells with the mutant o-ribosomes to the expression of the same reporter in OSYRIS cells containing o-ribosomes with wt 23S rRNA (100%). The respective wt 23S rRNA residues are indicated, and the identity of the assessed mutants are shown. High translation activity was defined as that where reporter expression was >60%. The gain-of-function mutants that combine high bypass score with high translation activity are shown by darker bars (see FIG. 4e and FIG. 13). The data represents the results of two independent biological replicates, and the error bars indicate the experimental error. The numeric data can be found in FIG. 17. The normalized fluorescence reading (relative fluorescence units over A₆₀₀) of OSYRIS cells containing o-ribosomes with wt 23S rRNA was taken as 100%.

FIG. 15. The termination stalling bypass scores of the individual PTC mutants. TnaC stalling bypass score was calculated as the ratio of GFP fluorescence (normalized by cell density) in OSYRIS cells expressing GFP-TnaC relative to that in cells with the GFP-TnaC(W12R) reporter. The bypass score for cells with o-ribosomes with wt 23S rRNA is 0.17. A threshold high bypass score (≥0.3, the dashed red line) was defined as that afforded by the U2609C mutation, which has been reported to diminish the translation arrest at the tnaC stop codon^{29,30}. The respective wt 23S rRNA residues are indicated and the identity of the assessed mutants are shown. The gain-of-function mutants that combine high bypass score with high translation activity (see FIG. 4e and FIG. 14) are shown in blue. The data represents the results of two independent biological replicates, and the error bars indicate the experimental error. n.s. indicates no statistical significance, * indicates p<0.05, ** indicates p<0.005, *** indicates p<0.0005 by Student's t-test, comparing the value of each mutant to the wt ribosomes. The numeric data can be found in FIG. 17.

FIG. 16. Testing the gain-of-function mutants in a cell-free translation system. a, Sucrose gradient fractionation under subunit-dissociation conditions of the ribosomal material from OSYRIS cells. The 30S and 50S subunits prepared from dissociated wt ribosomes (arrow) were used as markers. Gray shading indicates the 50S subunit fractions that were collected and used in the cell-free translation experiments. b, Analysis of the purity of the 50S material (isolated as described in A) by agarose gel electrophoresis of the rRNA. Wild type 16S and 23S, as well as purified Ribo-T rRNAs, were used as mobility markers. c, The principle (top) and results (bottom) of the in vitro toeprinting experiments. The tnaC template was translated by ribosomes assembled from the isolated mutant 50S subunits and wt 30S subunits. For each mutant, translation reactions were performed under three different conditions: (I) in the presence of the inhibitor L-PSA of prolyl-tRNA synthetase²¹ that stalls the ribosome with the tnaC Pro24 codon in the A site; the intensity of the corresponding toeprinting band (indicated by an open arrowhead) reflects the translation activity of the mutant ribosome; (H) in the presence of high concentration of L-tryptophan (5 mM) that leads to translation arrest at the stop codon (indicated by the green arrowhead) unless the rRNA mutation alleviates stalling; (L) at low concentration of L-tryptophan (5 μM), when no or limited stalling at the stop codon is detected. The termination stalling efficiency (FIG. 4f) was computed by as the ratio of the intensity of the stop codons toeprint bands of the H samples relative to those of the Pro24 codon in the I samples.

FIG. 17. Provides a Table showing the translational activity and termination stalling bypass score of the PTC library mutants described in Example 1.

FIG. 18. Provides a Table showing the genotypes of E. coli strains used in Example 1.

FIG. 19. Provides a Table showing primers used in Example 1.

FIG. 20. Provides a Table showing the genotype of the OSYRIS cells used in Example 1.

FIG. 21. Provides a Table showing primer and nucleotide combinations used for the primer extension analysis of Example 1.

FIG. 22. A) Secondary structure of a large subunit rRNA and a small subunit rRNA. B) Gene coding a large subunit rRNA and a small subunit rRNA.

FIG. 23. A) Tethered ribosome having a large subunit, a small subunit, and a linking moiety. B) Gene encoding the tethered ribosome of FIG. 23A.

FIG. 24. Permutation of a ribosome rRNA.

FIG. 25. A) Plasmid having a gene encoding for rRNA. B) Plasmid having a gene encoding for the rRNA of a tethered ribosome.

### DETAILED DESCRIPTION

Ribosomes with tethered and thus inseparable subunits (“Ribo-T”) that are capable of successfully carrying out protein synthesis are disclosed. Ribo-T may be prepared by engineering a ribosome comprising a small subunit, a large subunit, and a linking moiety that tethers the small subunit with the large subunit. The engineered ribosome may comprise a hybrid rRNA comprising a small subunit rRNA sequence, a large subunit rRNA sequence, and RNA linkers that may covalently link the small subunit rRNA sequence and the large subunit rRNA sequence into a single entity. The engineered ribosome may be prepared by expressing a polynucleotide encoding the rRNA of the engineered ribosome. The engineered ribosome may also be evolved by positively or negatively selecting mutations. Strikingly, Ribo-T is not only functional in vitro, but is able to support cell growth even in the absence of wild-type (“wt”) ribosomes. As a result, Ribo-T has many uses. For example, Ribo-T may be used to prepare sequence-defined polymers, such as naturally occurring proteins or unnaturally occurring amino-acid polymers; create fully orthogonal ribosome-mRNA systems in vitro or in vivo; explore poorly understood functions of the ribosome; and engineer ribosomes with new functions.

Tethered Ribosome

Reference is made to U.S. Publication No. 2017/0073381, which discloses tethered ribosomes and methods of making and using tethered ribosomes and which content is incorporated herein by reference in its entirety. The engineered ribosome comprises a small subunit, a large subunit, and a linking moiety, wherein the linking moiety tethers the small subunit with the large subunit. The engineered ribosome is capable of supporting translation of a sequence-defined polymer.

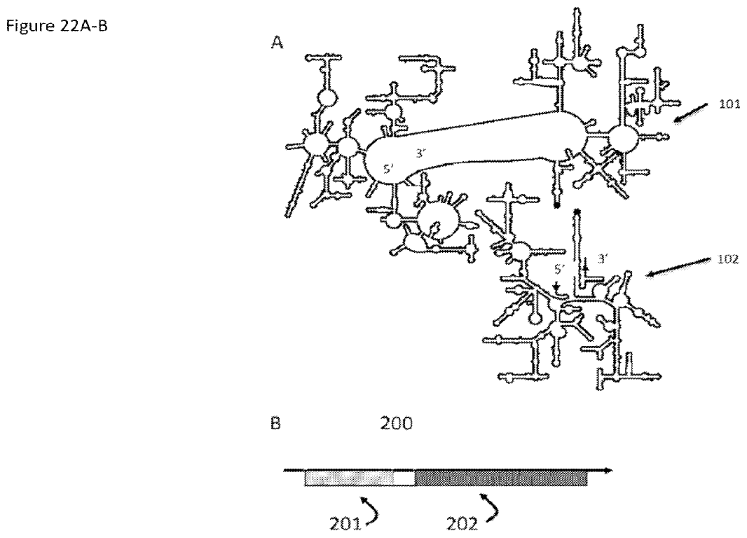

In contrast to a naturally occurring ribosome, the engineered ribosome has a large and a small subunit that are not separable. FIG. 22 depicts a portion of a wild-type ribosome having a small subunit and a large subunit that are separable. FIG. 22A illustrates the secondary structure of a large subunit rRNA 101 and a small subunit rRNA 102 that together form a portion of a functional ribosome. FIG. 22B illustrates an rRNA gene 200 comprising the operon encoding the large subunit rRNA 202 and the operon encoding the small subunit rRNA 201. In the wild-type rRNA, the large and small subunit rRNAs are excised from the primary transcript and processed to mature individual subunits.

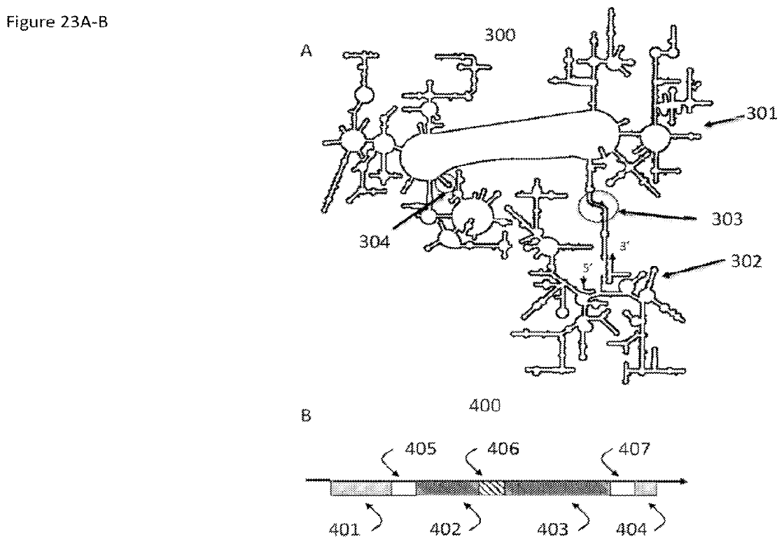

An embodiment of the engineered tethered ribosome is illustrated in FIG. 23. FIG. 23A illustrates the secondary structure of a portion of rRNA of the engineered ribosome 300. The engineered ribosome comprises a large subunit 301, a small subunit 302, and a linking moiety 303 that tethers the small subunit 302 with the large subunit 301. In the present example, the linking moiety 303 tethers the rRNA of the small subunit 302 with the rRNA of the large subunit 301. The engineered ribosome may also comprise a connector 304, that closes the ends of a native large subunit rRNA. FIG. 23B illustrates an example of an rRNA gene 400 and the operon encoding to the engineered ribosome 300.

Large Subunit

The large subunit 301 comprises a subunit capable of joining amino acids to form a polypeptide chain. The large subunit 301 may comprise a first large subunit domain (“L1 polynucleotide domain” or “L1 domain”), a second large subunit domain (“L2 polynucleotide domain” or “L2 domain”), and a connector domain (“C polynucleotide domain” or “C domain”) 304, wherein the L1 domain is followed, in order, by the C domain and the L2 domain, from 5′ to 3′.

FIG. 23B illustrates an example of an rRNA gene 400 that encodes the engineered ribosome 300, and provides an alternative representation for understanding the engineered ribosome. The encoding polynucleotide 400 may comprise difference sequences that encode for the various domains of the engineered ribosome 300. As illustrated in FIG. 23B, the polynucleotide encoding the large subunit rRNA 301 comprises the polynucleotide encoding the L1 domain 402, the polynucleotide encoding the C domain 406, and the polynucleotide encoding the L2 domain 403.

The large subunit rRNA 301 may be a permuted variant of a separable large subunit rRNA. In certain embodiments, the permuted variant is a circularly permuted variant of a separable large subunit rRNA. The separable large subunit may be any functional large subunit. In certain embodiments, the separable large subunit may be a 23S rRNA. In certain embodiments, the separable large subunit is a wild-type large subunit rRNA. In specific embodiments, the separable large subunit is a wild-type 23S rRNA.

If the large subunit 301 is a permuted variant of a large subunit rRNA, then the polynucleotide consisting essentially of the L2 domain followed by the L1 domain, from 5′ to 3′, may be substantially identical to a large subunit rRNA. In certain embodiments, the polynucleotide consisting essentially of the L2 domain followed by the L1 domain, from 5′ to 3′, is at least 90% identical, at least 91% identical, at least 92% identical, at least 93% identical, at least 94% identical, at least 95% identical, at least 96% identical, at least 97% identical, at least 98% identical, or at least 99% identical to the large subunit rRNA.

In certain embodiments where the large subunit 301 is a permuted variant of a separable large subunit rRNA, the large subunit 301 may further comprise a C domain 304 that connects the native 5′ and 3′ ends of the separable large subunit rRNA. The C domain may comprise a polynucleotide having a length ranging from 1-200 nucleotides. In certain embodiments, the C domain 304 comprises a polynucleotide having a length ranging from 1-150 nucleotides 1-100 nucleotides, 1-90 nucleotides, from 1-80 nucleotides, 1-70 nucleotides, 1-60 nucleotides, 1-50 nucleotides, 1-40 nucleotides, 1-30 nucleotides, 1-20 nucleotides, 1-10 nucleotides, 1-9 nucleotides, 1-8 nucleotides, 1-7 nucleotides, 1-6 nucleotides, 1-5 nucleotides, 1-4 nucleotides, 1-3 nucleotides, or 1-2 nucleotides. In certain embodiments, the C domain comprises a GAGA polynucleotide.

Small Subunit

The small subunit 302 is capable of binding mRNA. The small subunit 302 comprises a first small subunit domain (“S1 polynucleotide domain” or “S1 domain”) and a second small subunit domain (“S2 polynucleotide domain” or “S2 domain”), wherein the S1 domain is followed, in order, by S2 domain, from 5′ to 3′. Referring again to FIG. 23B, the polynucleotide encoding the small subunit rRNA 302 comprises the polynucleotide encoding the S1 domain 401 and the polynucleotide encoding the S2 domain 404.

The small subunit rRNA 302 may be a permuted variant of a separable small subunit rRNA. In certain embodiments, the permuted variant is a circularly permuted variant of a separable small subunit rRNA. The separable small subunit may be any functional small subunit. In certain embodiments, the separable small subunit may be a 16S rRNA. In certain embodiments, the separable small subunit is a wild-type small subunit rRNA. In specific embodiments, the separable small subunit is a wild-type 23S rRNA.

If the small subunit 302 is a permuted variant of a small subunit rRNA, then the polynucleotide consisting essentially of the S1 domain followed by the S2 domain, from 5′ to 3′, may be substantially identical to a small subunit rRNA. In certain embodiments, the polynucleotide consisting essentially of the S1 domain followed by the S2 domain, from 5′ to 3′, is at least 90% identical, at least 91% identical, at least 92% identical, at least 93% identical, at least 94% identical, at least 95% identical, at least 96% identical, at least 97% identical, at least 98% identical, or at least 99% identical to the small subunit rRNA.

The small subunit may further comprise a modified-anti-Shine-Dalgarno sequence. The modified anti-Shine-Dalgarno sequence allows for translation of templates having a complementary Shine-Dalgarno sequence different from an endogenous cellular mRNA.

Linking Moiety

Referring again to FIG. 23B, the linking moiety 303 tethers the small subunit 302 with the large subunit 301. In certain embodiments that linking moiety covalently bonds a helix of the large subunit 301 to a helix of the small subunit 302.

The linking moiety may also comprise a first tether domain (“T1 polynucleotide domain” or “T1 domain”) and a second tether domain (“T2 polynucleotide domain” or “T2 domain”). Referring again to FIG. 23B, the polynucleotide encoding the linking moiety 303 comprises the polynucleotide encoding the T1 domain 405 and the polynucleotide encoding the T2 domain 407.

The T1 domain links that S1 domain and the L1 domain, wherein the S1 domain is followed, in order, by the T1 domain and the L1 domain, from 5′ to 3′. The T1 domain may comprise a polynucleotide having a length ranging from 5-200 nucleotide, 5-150 nucleotides, 5-100 nucleotides, 5-90 nucleotide, 5-80 nucleotides, 5-70 nucleotides, 5-60 nucleotides, 5-50 nucleotides, 5-40 nucleotides, 5-30 nucleotides, or 5-20 nucleotides, including polynucleotides having 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides. In certain embodiments, T1 comprises polyadenine. In certain embodiments, T1 comprises polyuridine. In certain embodiments, T1 comprises an unstructured polynucleotide. In certain embodiments, T1 comprises nucleotides that base-pairs with the T2 domain.

The T2 domain links that L2 domain and the S2 domain, wherein the L2 domain is followed, in order, by the T2 domain and the S2 domain, from 5′ to 3′. The T2 domain may comprise a polynucleotide having a length ranging from 5-200 nucleotides, 5-150 nucleotides, 5-100 nucleotides, 5-90 nucleotide, 5-80 nucleotides, 5-70 nucleotides, 5-60 nucleotides, 5-50 nucleotides, 5-40 nucleotides, 5-30 nucleotides, or 5-20 nucleotides, including polynucleotides having 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides. In certain embodiments, T1 comprises polyadenine. In certain embodiments, T2 comprises polyuridine. In certain embodiments, T2 comprises an unstructured polynucleotide. In certain embodiments, T2 comprises nucleotides that base-pairs with the T1 domain.

In embodiments having a T1 domain and a T2 domain, the T1 domain and the T2 domain may have the same number of polynucleotides. In other embodiments, the T1 domain and the T2 domain may have a different number of polynucleotides.

In certain embodiments, the engineered ribosome may comprise a S1 domain followed, in order, by a T1 domain, a L1 domain, a C domain, a L2 domain, a T2 domain, and a S2 domain, from 5′ to 3′. In specific embodiments, the engineered ribosome may consist essentially of a S1 domain followed, in order, by a T1 domain, a L1 domain, a C domain, a L2 domain, a T2 domain, and a S2 domain, from 5′ to 3′.

Mutations

In certain embodiments, an engineered ribosome may comprise one or more mutations. In specific embodiments the mutation is a change-of-function mutation. A change-of-function mutation may be a gain-of-function mutation or a loss-of-function mutation. A gain-of-function mutation may be any mutation that confers a new function. A loss-of-function mutation may be any mutation that results in the loss of a function possessed by the parent.

In certain embodiments, the change-of-function mutation may be in the peptidyl transferase center of the ribosome. In specific embodiments, the change-of-function mutation may be in an A-site of the peptidyl transferase center. In other embodiments, the change-of-function mutation may be in the exit tunnel of the engineered ribosome.

In certain embodiments the change-of-function mutation may be an antibiotic resistance mutation. The antibiotic resistance mutation may be either in the large subunit or the small subunit. In certain embodiments antibiotic resistance mutation may render the engineered ribosome resistant to an aminoglycoside, a tetracycline, a pactamycin, a streptomycin, an edein, or any other antibiotic that targets the small ribosomal subunit. In certain embodiments antibiotic resistance mutation may render the engineered ribosome resistant to a macrolide, a chloramphenicol, a lincosamide, an oxazolidinone, a pleuromutilin, a streptogramin, or any other antibiotic that targets the large ribosomal subunit.

Designing the Tethered Ribosome

A successful chimeric construct that tethers a large subunit and a small subunit must i) properly interact with the ribosomal proteins and biogenesis factors for functional ribosome assembly; ii) avoid ribonuclease degradation; and iii) have a linker(s) sufficiently short to ensure subunit cis-association, yet long enough for minimal inhibition of subunit movement required for translation initiation, elongation, and peptide release. The native ends of the large subunit and the small subunit are unsuitable given the design constraints outlined above. For example, in a native prokaryotic ribosome, for example, the 5′ and 3′ ends of 16S and 23S rRNA are too far apart (>170 Å) to be connected with a nuclease resistant RNA linker. As a result, alternative designs are needed if functioning engineered ribosome are to be realized.

One approach for designing a tethered ribosome is to permute a large subunit to generate new 5′ and 3′ termini. In certain embodiments, a circular permutation (CP) approach is employed because the native ends on the large subunit are proximal to each other. Circular permutation can be illustrated in the following scheme:

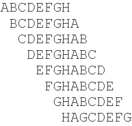

As such, in circular permutations of a polynucleotide, the sequence of the polynucleotide is maintained in each permutation but each nucleotide is at the end of an individual permutation. Circular permutations are utilized to replace the end of a polynucleotide at a different position while maintaining the secondary structure of the polynucleotide.

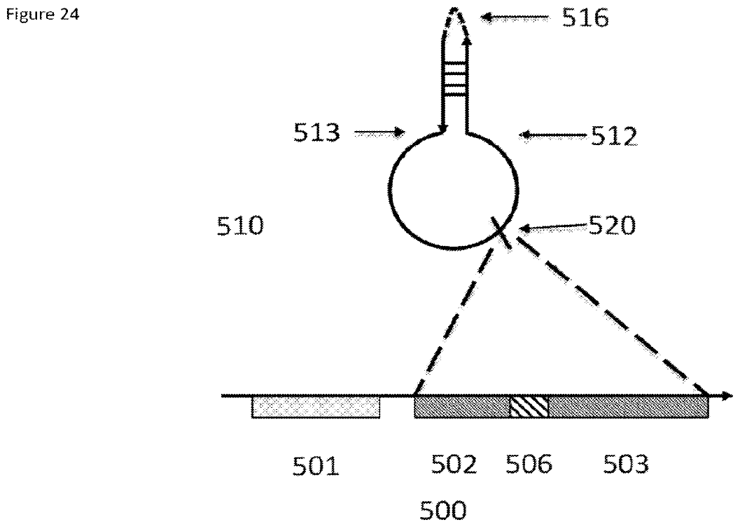

The CP approach has been pioneered in vitro by Polacek and coworkers [Erlacher 2005], and a subsequent pilot study showed that three 23S rRNA circularly permuted variants could assemble into a functional subunit in vivo [Kitahara 2009]. This approach is illustrated in FIG. 24. In FIG. 24, a native large subunit ribosome 510 comprises a second large subunit domain (L2 domain) 513 followed by a first large subunit domain (L1 domain), from 5′ to 3′. The native ends of a large subunit ribosome 510 (which is a simplified representation of the large subunit rRNA 101 represented in FIG. 22A) are connected through a connector domain (C domain) 511 and new termini are prepared at 512. The permuted subunit prepared by this approach comprises the first large subunit domain (L1 domain), followed, in order, by the connector domain (C domain) and the second large subunit domain (L2 domain), from 5′ to 3′. FIG. 24 also illustrates a portion of a gene 500 that encodes for the small subunit 501 and the new permuted large subunit comprising the L1 domain 502, followed, in order, by the C domain 506 and the L2 domain 503, from 5′ to 3′.

Continuing the approach outlined above, new termini for the small subunit need to be prepared so that the new termini for the small unit can be joined with the new termini of the large subunit by the linking moiety, as shown in FIG. 23A, B.

The approach outlined above can be used to generate collections of circularly permuted mutants with new termini. The new termini may be prepared at any location in the native subunit. Although some new termini result in permuted mutants may not be viable, the process disclosed herein is capable of generating and testing collections of permuted mutants.

In some embodiments, the location of the new termini of a small subunit or large subunit may be selected based on the secondary structure of a subunit, the proximity to the other subunit, the ribosome viability, or any combination thereof.

The secondary structure of either or both of the large subunit and the small subunit may be used to determine the location for new termini. In certain embodiments, the new termini are prepared in a helix of a native subunit. In some specific embodiments the new termini are prepared in hairpin of a native subunit.

The proximity to the other subunit may be used to select the location of the new termini in either or both of the large subunit or the small subunit. In certain embodiments, the new termini are located in the subunit solvent side of the native subunit. In some other embodiments the new termini are located close to the subunit interface rim. In certain specific embodiments the new termini are located in the subunit solvent side and close to the subunit interface rim.

Ribosome viability may be used to select the location of the new termini in either or both of the large subunit or the small subunit. For example, polynucleotide sequences or secondary structures that are in either or both of the large subunit or the small subunit that are not highly conserved in populations may be used to select the location for new termini.

In certain embodiments where the engineered ribosome is a 23S construct, the linking moiety may covalently bond helix 10, helix 38, helix 42, helix 54, helix 58, helix 63, helix 78, or helix 101 of a permuted variant of the 23S rRNA. In certain embodiments where the engineered ribosome is a 16S rRNA construct, the linking moiety may covalently bond helix 11, helix 26, helix 33, or helix 44 of a permuted variant of the 16S rRNA. In certain other embodiments where the engineered ribosome is a 16S construct, the linking moiety may covalently bond close to the E-site of a permuted variant of the 16S rRNA. In specific embodiments where the engineered ribosome is a 16S-23S construct, the linking moiety may covalently bond helix 44 of a permuted variant 16S rRNA with helix 101 of a permuted variant 23S rRNA, the linking moiety may covalently bond helix 26 of a permuted variant 16S rRNA with helix 10 of a permuted variant 23S rRNA, the linking moiety may covalently bond helix 33 of a permuted variant 16S rRNA with helix 38 of a permuted variant 23S rRNA, the linking moiety may covalently bond helix 11 of a permuted variant 16S rRNA with helix 58 of a permuted variant 23S rRNA, the linking moiety may covalently bond helix 44 of a permuted variant 16S rRNA with helix 58 of a permuted variant 23S rRNA, the linking moiety may covalently bond helix 26 of a permuted 1 variant 6S rRNA with helix 54 of a permuted variant 23S rRNA, the linking moiety may covalently bond helix 11 of a permuted variant 16S rRNA with helix 63 of a permuted variant 23S rRNA, or the linking moiety may covalently bond helix 44 of a permuted variant 16S rRNA with helix 63 of a permuted variant 23S rRNA.

As explained above, the linking moiety must be sufficiently short to prevent degradation and to ensure subunit cis-association while long enough for minimal inhibition of subunit movement required for translation initiation, elongation, and peptide release. As a result, the linking moiety must span tens of Angstroms between the new termini on the large subunit and the short subunit.

Polynucleotides Encoding the Tethered Ribosome

Polynucleotides encoding the tethered ribosome are also disclosed. The polynucleotide encoding for the tethered ribosome may be any polynucleotide capable of being expressed to produce the rRNA of the tethered ribosome. FIG. 23B illustrates a polynucleotide for preparing the rRNA of the tethered ribosome. The polynucleotide 400 comprises a sequence that encodes for the rRNA of a S1 domain 401 followed, in order, by a sequence that encodes for the rRNA of a T1 linker 405, a sequence that encodes for the rRNA of a L1 domain 402, a sequence that encodes for the rRNA of a C domain 406, a sequence that encodes for the rRNA of a L2 domain 403, a sequence that encodes for the rRNA of a T2 linker 407, and a sequence that encodes for the rRNA of a S2 domain 404, from 5′ to 3′.

The polynucleotides encoding for the tethered ribosome may further comprise genes encoding for other rRNA subunits of the ribosome or ribosomal proteins. For example, the polynucleotide encoding for an engineered ribosome comprising a permuted 23S rRNA tethered to a permuted 16S rRNA, the polynucleotide may further comprise a gene encoding for a 5S rRNA.

In certain embodiments the polynucleotide is a vector that may introduce foreign genetic material into a host cell. The vector may be a plasmid, viral vector, cosmid, or artificial chromosome.

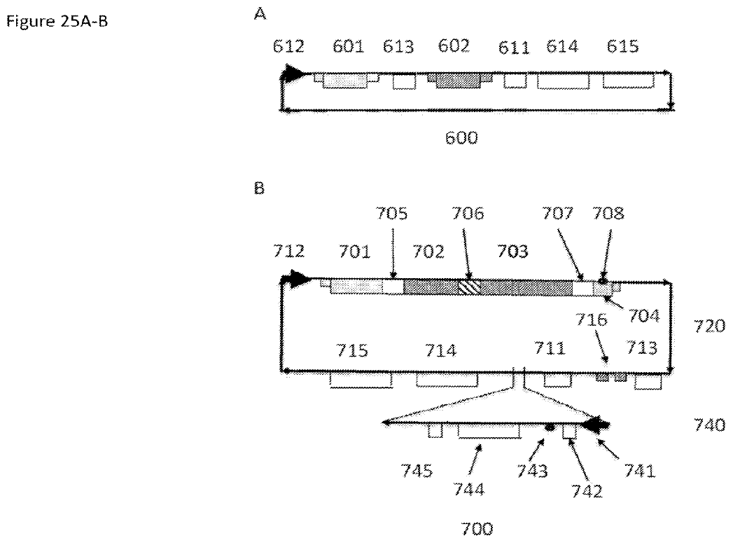

FIGS. 25A, B provide examples of plasmids that encode for a prokaryotic ribosome having separable subunits (FIG. 25A) and a polynucleotide encoding for a tethered ribosome (FIG. 25B). In a FIG. 25A, the plasmid 600 comprises a promoter 612, a gene encoding for a 16S subunit 601, including a representation of the processing stems indicated by the smaller rectangles, a tRNA gene 613, a gene encoding a 23S subunit 602, including a representation of the processing stems indicated by the smaller rectangles, a gene encoding a 5S subunit 611, a gene encoding antibiotic resistance 614, and a origin of replication gene 615. In some embodiments, the 16S subunit 601, includes a modified anti-Shine-Dalgarno sequence. The modified anti-Shine-Dalgarno sequence may be located in either of the small subunit domains, i.e., S1 or S2.

Optionally the plasmid encoding a prokaryotic ribosome having separable subunits comprises one or more additional genes. The additional gene(s) may comprise a modified Shine-Dalgarno sequence that is complimentary with a modified anti-Shine-Dalgarno sequence of the small subunit of the untethered ribosome.

In contrast to the plasmid encoding the ribosome having separable subunits, the plasmid encoding a tethered ribosome 700 has a chimeric gene encoding for a large subunit, a small subunit, and a linking moiety connecting the large subunit with the small subunit 701-707. Plasmid comprises the genes for the expression of the tethered ribosome 720. Optionally, the plasmid may further comprise one or more addition genes 740.

The gene encoding for the tethered subunits comprises the sequence that encodes for the rRNA of a S1 domain 701 followed, in order, by a sequence that encodes for the rRNA of a T1 linker 705, a sequence that encodes for the rRNA of a L1 domain 702, a sequence that encodes for the rRNA of a C domain 706, a sequence that encodes for the rRNA of a L2 domain 703, a sequence that encodes for the rRNA of a T2 linker 707, and a sequence that encodes for the rRNA of a S2 domain 704, from 5′ to 3′. The processing sequences of a small subunit flanking the chimeric gene, indicated by the small rectangles, may be retained for proper maturation of the small subunit termini, whereas the processing sequences for the large subunit 716 may be moved to another location in the plasmid or eliminated entirely to prevent cleavage of the large subunit out of the hybrid.

In certain embodiments, the plasmid encoding the tethered subunits further comprises a gene encoding a 5S subunit 711, a gene encoding antibiotic resistance 714, and an origin of replication gene 715.

Optionally, the plasmid encoding the tethered subunits may comprise a modified anti-Shine-Dalgarno sequence 708 (circle). Although the modified anti-SD sequence is shown in FIG. 25B to be located within the sequence encoding the S2 domain, the modified anti-Shine Dalgarno sequence may be located in either of the small subunit domains, i.e. S1 or S2. In some embodiments, a plasmid including tethered subunits comprise a wild-type anti-Shine-Dalgarno sequence.

Optionally, the plasmid encoding the tethered subunits comprises one or more additional genes 740. The additional gene may comprise a modified Shine-Dalgarno sequence that is complimentary with a modified anti-Shine-Dalgarno sequence of the tethered ribosome. In certain embodiments that additional gene may be a reporter gene. In specific embodiments, the reporter gene is a green fluorescent protein. In some embodiments, the additional gene comprise a wild-type anti-Shine-Dalgarno sequence.

Preparing the Polynucleotide

Methods of preparing the polynucleotide are also disclosed herein. The method comprises preparing a plasmid encoding a permuted subunit rRNA construct, identifying a viable permuted subunit rRNA construct, and preparing a polynucleotide encoding the engineered ribosome comprising a large subunit, a small subunit, and a linking moiety that tethers the small subunit with the large subunit.

Preparation of a plasmid encoding a permuted subunit rRNA construct may be accomplished by the circular permutation approach that connects the native ends of the subunit and prepares new termini FIG. 24. Preparation of the plasmid may comprise the steps of template preparation, plasmid backbone preparation, and assembly. The template preparation step may be accomplished by plasmid digestion and ligation. By way of example, a CP23S template may be prepared from pCP23S-EagI plasmid by EagI digestion and ligation. Each CP23S variant is generated by PCR using a circularized 23S rRNA gene as a template and a unique primer pair, with added sequences overlapping the destination plasmid backbone. The plasmid backbone preparation step may be accomplished by digestion of a plasmid with a restriction enzyme that linearized the backbone at the subunit processing stem site. By way of example, Plasmid backbone is prepared by digestion of pAM552-23S-AflII with AflII restriction enzyme, which linearizes the backbone at the 23S processing stem site. The assembly step incorporates the template with the plasmid backbone to prepare the plasmid encoding the permuted subunit rRNA. The assembly step may be accomplished by Gibson assembly.

To identify permuted subunit rRNA viable constructs, the plasmid encoding the permuted subunit rRNA may be introduced in to host cell strains and a screening mechanism is used to identify transformants. The host cells comprise the plasmid as well as a plasmid encoding for the wild-type rRNA operon and may be spotted onto an agar plate along with an antibiotic. The selection mechanism includes identifying transformants resistant to the antibiotic. By way of example, the plasmids may be transformed into Δ7 rrn SQ171 strain carrying pCSacB plasmid with wild-type rRNA operon and transformants resistant to ampicillin, erythromycin and sucrose are selected. To confirm complete replacement of the wild-type rRNA operon with the plasmid encoding for the permuted subunit rRNA, a three-primer diagnostic PCR check may be performed on the total plasmid extract.

Preparing a polynucleotide encoding the engineered ribosome comprising a large subunit, a small subunit, and a linking moiety that tethers the small subunit with the large subunit comprises grafting the permuted subunit rRNA construct and the linking moiety into the other subunit. In certain embodiments the preparation step may also include preparing a plasmid comprising the polynucleotide encoding the engineered ribosome comprising a large subunit, a small subunit, and a linking moiety that tethers the small subunit with the large subunit. In other embodiments, the preparation step may also include preparing a plasmid comprising the polynucleotide encoding the engineered ribosome comprising a large subunit, a small subunit, and a linking moiety that tethers the small subunit with the large subunit and a polynucleotide encoding for an additional gene.

Preparing the Tethered Ribosome

Also disclosed are methods for preparing the tethered ribosome. The tethered ribosome may be prepared by expressing a polynucleotide encoding the engineered ribosome. In certain embodiments preparation of the tethered ribosome further comprises preparing the polynucleotide encoding the engineered ribosome. In other embodiments the preparation of the tethered ribosome further comprises transforming a cell with the polynucleotide encoding the engineered ribosome. In some specific embodiments, the preparation of the tethered ribosome further comprises preparing the polynucleotide and transforming a cell with the polynucleotide.

Tethered Ribosome Evolution

Also disclosed are methods for evolving the tethered ribosome. Methods for tethered ribosome evolution include expressing a polynucleotide encoding for the engineered ribosome and selecting a mutant. The selection step may comprise a negative selection step, a positive selection step, or both a negative and a positive selection step. The mutant selected may comprise a tethered ribosome having a change-of-function mutation. The change-of-function mutation may be a gain-of-function mutation or a loss-of-function mutation.

Utility and Applications of Tethered Ribosomes

Some uses and applications of the tethered ribosomes are described below.

Artificial Cells

Artificial cells are disclosed. The artificial cell may comprise a polynucleotide encoding an engineered ribosome, the engineered ribosome comprising a small subunit, a large subunit, and a linking moiety, wherein the linking moiety tethers the small subunit with the large subunit. The artificial cell comprising a polynucleotide encoding the engineered ribosome may be capable of expressing the polynucleotide to prepare the engineered ribosome. In other embodiments, the artificial cell comprises the engineered ribosome. In some specific embodiments the artificial cell comprises a polynucleotide encoding the engineered ribosome and the engineered ribosome.

Artificial cells may comprise one or more translation mechanism. In a first embodiment, the artificial cell has one translation mechanism comprising an engineered ribosome, the engineered ribosome comprising a small subunit, a large subunit, and a linking moiety, wherein the linking moiety tethers the small subunit with the large subunit.

In another embodiment, the artificial cell may comprise two translation mechanisms. The first translation mechanism may comprise a ribosome wherein the ribosome lacks a linking moiety between the large subunit and the small subunit. The second translation mechanism comprises an engineered ribosome, the engineered ribosome comprising a small subunit, a large subunit, and a linking moiety, wherein the linking moiety tethers the small subunit with the large subunit. In some embodiments the first translation mechanism or the second translation mechanism is an orthogonal translation mechanism. In some embodiments the first translation mechanism and the second translation mechanism are orthogonal translation mechanisms. An orthogonal translation mechanism may be prepared by modifying the anti-Shine Dalgarno sequence of the ribosome to permit translation of templates having a complementary Shine-Dalgarno sequences different from the endogenous cellular mRNAs.

In another embodiment, a cell comprising a first mechanism and a second mechanism for protein translation is disclosed. The first mechanism comprises tethered ribosomes with a wild-type anti-Shine-Dalgarno sequence, wherein mRNA is translated by the ribosomes in accordance with the natural genetic code (that is, triplet code endogenous to the cell). The second mechanism includes an artificial mechanism derived from untethered ribosomes that functions to allow for expression of a heterologous gene. The second mechanism, in some embodiments, comprises ribosomes having a modified anti-Shine-Dalgarno sequence.

Preparation of Sequence-Defined Polymers

Methods for preparing sequence-defined polymers are also provided. In certain embodiments the method for preparing a sequence defined polymer comprises providing an engineered ribosome and providing an mRNA or DNA template encoding the sequence-defined polymer. In some embodiments, the engineered ribosome comprises a small subunit, a large subunit, and a linking moiety and wherein the linking moiety tethers the small subunit with the large subunit, and wherein the engineered ribosome comprises a modified anti-Shine-Dalgarno sequence. In some embodiments, the engineered ribosome comprises a small subunit, a large subunit, no linking moiety, and a modified Shine-Dalgarno sequence. In one aspect of the method, one of any of the steps includes adding at least one exogenous DNA template encoding an mRNA for the sequence-defined polymer.

In one aspect of the method, the sequence-defined polymer is a natural biopolymer. In another aspect of the method, the sequence-defined polymer is a non-natural biopolymer. In certain embodiments, the sequence-defined polymer comprises an amino acid. In certain embodiments the amino acid may be a natural amino acid. As used herein a natural amino acid is a proteinogenic amino acid encoded directly by a codon of the universal genetic code. In certain embodiments the amino acid may be an unnatural amino acid. As used here an unnatural amino acid is a nonproteinogenic amino acid. Examples of unnatural amino acids include, but are not limited to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl)alanine, a 3-methyl-phenylalanine, an O-4-allyl-L-tyrosine, a 4-propyl-L-tyrosine, a tri-O-acetyl-GlcNAcpβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan. In certain embodiments the sequence-defined polymer is a polypeptide or protein.

In one aspect of the method, the tethered subunit arrangement comprises a linking moiety between the 23S and 16S rRNAs. In one respect of this aspect, the linking moiety covalently bonds helix 101 of the 23S rRNA to helix 44 of the 16S rRNA. In another respect of this aspect, the linking moiety comprises a polynucleotide having a length ranging from 5 nucleotides to 200 nucleotides. The linked ribosome can further include an engineered 16S rRNA having a modified anti-Shine-Dalgarno sequence to permit translation in vitro of translation templates having a complementary SD sequence differing from endogenous cellular mRNAs. In this way, selective translation in vitro of mRNA to produce sequence defined biopolymers with high efficiency is possible.

In one aspect of the method, an engineered ribosome is untethered, and comprises a modified anti-Shine-Dalgarno (SD) 16S sequence to permit translation in vitro or in vivo of translation templates having a complementary SD sequence differing from endogenous cellular mRNAs. In this way, selective translation of mRNA to produce sequence defined biopolymers with high efficiency is possible.

In one aspect of the method, the mRNA or DNA template encodes a modified Shine-Dalgarno sequence. In certain embodiments the engineered ribosome comprises an anti-Shine-Dalgarno sequence complementary to the Shine-Dalgarno sequence encoded by the mRNA or DNA template.

In some embodiments, the mRNA or DNA template is provided to a modified cell (e.g., a cell comprising two different protein translation mechanisms), an extract from such a cell, or a purified translation system from such a cell.

Sequence-defined polymers may be prepared in vitro. In some embodiments, the method for preparing a sequence-defined polymer in vitro further comprises providing a ribosome-depleted cellular extract or a purified translation system. In certain embodiments, the ribosome-depleted cellular extract comprises an S150 extract prepared from mid- to late-exponential growth phase cell cultures or cultures having an O.D.600˜3.0 at time of harvest. In one aspect of the method, the ribosome-depleted extract is prepared with one or more polyamines, such as spermine, spermidine and putrescine, or combinations thereof. In one aspect of the method, the ribosome-depleted extract is prepared with a concentration of salts from about 50 mM to about 300 mM.

The preparation of ribosome-depleted cellular extracts and methods of using them for supporting translation in vitro of sequence-defined polymers is disclosed in International Patent Application No. PCT/US14/35376 to Michael Jewett et al., entitled IMPROVED METHODS FOR MAKING RIBOSOMES, filed Apr. 24, 2014, the contents of which are incorporated by reference herein in its entirety.

In one aspect of the method, mRNA encodes a modified Shine-Dalgarno sequence differing from endogenous cellular mRNAs present in the ribosome-depleted cellular extract. In one respect of this aspect, an engineered ribosome includes an altered 16S rRNA having a modified anti-Shine-Dalgarno sequence complementary to the modified Shine-Dalgarno sequence to permit translation in vitro of the mRNA to prepare the sequence defined biopolymer in vitro.

In one aspect, the method is configured for fed-batch operation or continuous operation. In another aspect of the method, at least one substrate is replenished during operation.

In one aspect of the method, at least one step includes a DNA-dependent RNA polymerase. In one aspect of the method, at least one macromolecular crowding agent is included in one of the steps. In one aspect of the method, at least one reducing agent (e.g., dithiothreitol, tris(2-carboxyethyl) phosphine hydrochloride, etc.) is included in one of the steps.

Sequence-defined polymers may be prepared in vivo. The method for preparing a sequence-defined polymer in vivo may occur in an artificial cell as disclosed above. The artificial cell may have a translation mechanism comprising an engineered ribosome, wherein the engineered ribosome comprises a small subunit, a large subunit, and a linking moiety and wherein the linking moiety tethers the small subunit with the large subunit. In certain embodiments the artificial cell has one translation mechanism. In other embodiments the cell has two translations mechanisms. In some embodiments, the cell has two protein translations mechanisms, the first protein translation mechanism comprising ribosomes, wherein the ribosomes lack a linking moiety between the large subunit and the small subunit and the second protein translation mechanism comprises ribosomes, wherein the ribosomes include a linking moiety linking the large subunit and the small subunit. In some embodiments, the ribosomes of the first translations system comprises a modified anti-Shine-Dalgarno sequence and the ribosomes of the second translation system include a wild-type (unmodified) anti-Shine-Dalgarno sequence.

### Terminology

Unless defined otherwise, all technical and scientific terms used herein have the same meaning as commonly understood by one of skill in the art to which the invention pertains. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

A range includes each individual member. Thus, for example, a group having 1-3 members refers to groups having 1, 2, or 3 members.

It should also be understood that, unless clearly indicated to the contrary, in any methods claimed herein that include more than one step or act, the order of the steps or acts of the method is not necessarily limited to the order in which the steps or acts of the method are recited.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “nucleic acid” and “oligonucleotide,” as used herein, refer to polydeoxyribonucleotides (containing 2-deoxy-DRibose), polyribonucleotides (containing DRibose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

A “fragment” of a polynucleotide is a portion of a polynucleotide sequence which is identical in sequence to but shorter in length than a reference sequence. A fragment may comprise up to the entire length of the reference sequence, minus at least one nucleotide. For example, a fragment may comprise from 5 to 1000 contiguous nucleotides of a reference polynucleotide. In some embodiments, a fragment may comprise at least 5, 10, 15, 20, 25, 30, 40, 50, 60, 70, 80, 90, 100, 150, 250, or 500 contiguous nucleotides of a reference polynucleotide. Fragments may be preferentially selected from certain regions of a molecule. The term “at least a fragment” encompasses the full length polynucleotide. A “variant,” “mutant,” or “derivative” of a reference polynucleotide sequence may include a fragment of the reference polynucleotide sequence.

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)n sequence, where n is in the range from about 20 to about 200). The region of the primer that is sufficiently complementary to the template to hybridize is referred to herein as the hybridizing region.

The term “promoter” refers to a cis-acting DNA sequence that directs RNA polymerase and other trans-acting transcription factors to initiate RNA transcription from the DNA template that includes the cis-acting DNA sequence.

The terms “target, “target sequence”, “target region”, and “target nucleic acid,” as used herein, are synonymous and refer to a region or sequence of a nucleic acid which is to be amplified, sequenced or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

As used herein, the term “sequence defined polymer” refers to a polymer having a specific primary sequence. A sequence defined polymer can be equivalent to a genetically-encoded defined polymer in cases where a gene encodes the polymer having a specific primary sequence.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, “expression template” refers to a nucleic acid that serves as substrate for transcribing at least one RNA that can be translated into a polypeptide or protein. Expression templates include nucleic acids composed of DNA or RNA. Suitable sources of DNA for use a nucleic acid for an expression template include genomic DNA, plasmid DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

As used herein, “tethered,” “conjoined,” “linked,” “connected,” “coupled” and “covalently-bonded” have the same meaning as modifiers.

As used herein, “tethered ribosome,” and “Ribo-T” will be used interchangeably.

As used herein, the term “engineered ribosome” refers to a ribosome that has been modified. Exemplary modifications may include, but are not limited to one or more of tethering subunits, altering or subunits, and altering one or more rRNA sequence. Exemplary, non-limiting modification may include one or more of a modified: 16S rRNA; 23S rRNA; anti-Shine-Dalgarno sequence, peptidyl transferase center; nascent exit tunnel; ecoding center of the ribosome; interaction site with elongation factors; tRNA binding site; chaperone binding site; nascent chain modifying enzyme binding sites; GTPase center; introduction of antibiotic resistance sequence, etc.).

As used herein, the term “wild-type,” “native,” or “endogeneous” refer to a substance or condition typically found in a given organism.

As used herein, the term “mutant,” exogenous,” “orthagnol,” and “non-native” refer to a substance or conditions typically not found in a given organism.

As used here, “CP” refers to a circularly permuted subunit. As used herein, when CP is followed by “23S” that refers to a circularly permuted 23S rRNA. As used herein, when CP followed by a number may refer to the location of the new 5′ end in a secondary structure, e.g. CP101 means the new 5′ end is in helix 101 of the 23S rRNA, or to the location of the new 5′ nucleotide, e.g. CP2861 means the new 5′ nucleotide is the nucleotide 2861 of the 23 rRNA, depending on context.

As used herein, “translation template” refers to an RNA product of transcription from an expression template that can be used by ribosomes to synthesize polypeptide or protein.

As used herein, a “ribosomal binding site” or “RBS” is a sequence of nucleotides upstream of the start codon of an mRNA transcript that is responsible for the recruitment of a ribosome during the initiation of protein translation. The RBS may include the Shine-Dalgarno sequence. The Shine-Dalgarno (SD) sequence is a ribosomal binding site in prokaryotic messenger RNA, which generally is located approximately 8 bases upstream of the start codon AUG. The SD sequence helps recruit the ribosome to the messenger RNA (mRNA) to initiate protein synthesis by aligning the ribosome with the start codon. The six-base consensus sequence is AGGAGG and in E. coli the sequence is AGGAGGU.

Miscellaneous

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Illustrative Embodiments

The following embodiments are illustrative and should be interpreted to limit the scope of the claimed subject matter.

Embodiment 1. An engineered ribosome, the engineered ribosome comprising a small subunit, a large subunit, and a linking moiety, a. wherein the linking moiety tethers the small subunit with the large subunit and b. wherein the engineered ribosome is capable of supporting translation of a sequence defined polymer.

Embodiment 2. The engineered ribosome of embodiment 1, wherein the small subunit comprises rRNA and protein, wherein the large subunit comprises rRNA and protein, and wherein the linking moiety tethers the rRNA of the small subunit with the rRNA of the large subunit.

Embodiment 3. The engineered ribosome of embodiment 1 or 2, wherein the large subunit comprises a permuted variant of a 23S rRNA (e.g., a circularly permuted variant of 23 rRNA).

Embodiment 4. The engineered ribosome of any of embodiments 1-3, wherein the small subunit comprises a permuted variant of a 16S rRNA (e.g., a circularly permuted variant of 23 rRNA).

Embodiment 5. The engineered ribosome of any of embodiments 1-4, wherein the small subunit comprises a modified anti-Shine-Dalgarno sequence to permit translation of templates having a complementary Shine-Dalgarno sequence different from endogenous cellular mRNAs (e.g., wherein the modified anti-Shine-Dalgarno sequence of the small subunit is complementary to the Shine-Dalgarno sequence different from endogenous cellular mRNAs).

Embodiment 6. The engineered ribosome of any of embodiments 1-5, wherein the linking moiety covalently bonds a helix of the large subunit to a helix of the small subunit.

Embodiment 7. The engineered ribosome of any of embodiments 3-6, wherein the linking moiety covalently bonds helix 10, helix 38, helix 42, helix 54, helix 58, helix 63, helix 78, or helix 101 of the permuted variant of the 23S rRNA.

Embodiment 8. The engineered ribosome of any of embodiments 4-7, wherein the linking moiety covalently bonds helix 11, helix 26, helix 33, or helix 44 of the permuted variant of the 16S rRNA.

Embodiment 9. The engineered ribosome of any of embodiments 1-8, wherein the large subunit comprises or consists essentially of a L1 polynucleotide domain (e.g., a fragment of 23S rRNA), a L2 polynucleotide domain (e.g., a fragment of 23S rRNA), and a C polynucleotide domain, wherein the L1 domain is followed, in order, by the C domain and the L2 domain, from 5′ to 3′.

Embodiment 10. The engineered ribosome of embodiment 9, wherein the polynucleotide comprising or consisting essentially of the L2 domain followed by the L1 domain, from 5′ to 3′, is substantially identical to 23S rRNA or a fragment of 23S rRNA.

Embodiment 11. The engineered ribosome of embodiment 9 or 10, wherein the polynucleotide comprising or consisting essentially of the L2 domain followed by the L1 domain, from 5′ to 3′, is at least 95% identical to 23S rRNA or a fragment of 23S rRNA (or at least 96%, 97%, 98%, or 99% identical to 23S rRNA or a fragment of 23S rRNA).

Embodiment 12. The engineered ribosome of any of embodiments 9-11, wherein the C domain comprises a polynucleotide having a length ranging from 1-200 nucleotides.

Embodiment 13. The engineered ribosome of any of embodiments 9-12, wherein the C domain comprises a GAGA polynucleotide.

Embodiment 14. The engineered ribosome of any of embodiments 1-13, wherein the small subunit comprises or consists essentially of a S1 polynucleotide domain (e.g., a fragment of 16S rRNA) and a S2 polynucleotide domain (e.g., a fragment of 16S rRNA), wherein the S1 domain is followed, in order, by the S2 domain, from 5′ to 3′.

Embodiment 15. The engineered ribosome of embodiment 14, wherein the polynucleotide comprising or consisting essentially of the S1 domain followed by the S2 domain, from 5′ to 3′, is substantially identical to a 16S rRNA (or a fragment of 16S rRNA).

Embodiment 16. The engineered ribosome of embodiment 14 or 15, wherein the polynucleotide comprising or consisting essentially of the S1 domain followed by the S2 domain, from 5′ to 3′, is at least 95% identical to a 16S rRNA (or at least 96%, 97%, 98%, or 99% identical to 23S rRNA or a fragment of 23S rRNA).

Embodiment 17. The engineered ribosome of any of embodiments 1-16, wherein the linking moiety comprises a T1 polynucleotide domain and a T2 polynucleotide domain.

Embodiment 18. The engineered ribosome of embodiment 17, wherein the T1 domain links the S1 domain and the L1 domain and wherein the S1 domain is followed, in order, by the T1 domain and the L1 domain, from 5′ to 3′.

Embodiment 19. The engineered ribosome of embodiment 17 or 18, wherein the T1 domain comprises a polynucleotide having a length ranging from 5 to 200 nucleotides.

Embodiment 20. The engineered ribosome of embodiment 19, wherein the T1 domain comprises a polynucleotide having a length ranging from 7 to 20 nucleotides.

Embodiment 21. The engineered ribosome of any of embodiments 17-20, wherein the T1 domain comprises a polyadenine polynucleotide.

Embodiment 22. The engineered ribosome of any of embodiments 17-20, wherein the T1 domain comprises a polyadenine polynucleotide having a length of 7 to 12 adenine nucleotides.

Embodiment 23. The engineered ribosome of any of embodiments 17-22, wherein the T2 domain links the S2 domain and the L2 domain and wherein the L2 domain is followed, in order, by the T2 domain and the S2 domain, from 5′ to 3′.

Embodiment 24. The engineered ribosome of any of embodiments 17-24, wherein the T2 domain comprises a polynucleotide having a length ranging from 5 to 200 nucleotides.

Embodiment 25. The engineered ribosome of embodiment 17, 23, or 24, wherein the T2 domain comprises a polynucleotide having a length ranging from 7 to 20 nucleotides.

Embodiment 26. The engineered ribosome of any of embodiments 17-25, wherein the T2 domain comprises a polyadenine polynucleotide.

Embodiment 27. The engineered ribosome of any of embodiments 17-26, wherein the T2 domain comprises a polyadenine polynucleotide having a length of 7 to 12 adenine nucleotides.

Embodiment 28. The engineered ribosome of any of embodiments 17-27, wherein the ribosome comprises the S1 domain followed, in order, by the T1 domain, the L1 domain, the C domain, the L2 domain, the T2 domain, and the S2 domain, from 5′ to 3′.

Embodiment 29. The engineered ribosome of any of embodiments 17-28, wherein the ribosome comprises a polynucleotide consisting essentially of the S1 domain is followed, in order, by the T1 domain, the L1 domain, the C domain, the L2 domain, the T2 domain, and the S2 domain, from 5′ to 3′.

Embodiment 30. The engineered ribosome of any of embodiments 1-29, wherein the engineered ribosome comprises a mutation.

Embodiment 31. The engineered ribosome of embodiment 30, wherein the mutation is a change-of-function mutation.

Embodiment 32. The engineered ribosome of embodiment 31, wherein the change-of-function mutation is in a peptidyl transferase center.

Embodiment 33. The engineered ribosome of embodiment 31, wherein the change-of-function mutation is in an A-site of the peptidyl transferase center.

Embodiment 34. The engineered ribosome of embodiment 31, wherein the change-of-function mutation is in one or more of the exit tunnel of the engineered ribosome, the interaction site with the translocon, or the interaction sites with the auxiliary proteins facilitating translation.

Embodiment 35. The engineered ribosome of any of embodiments 1-35, wherein the engineered ribosome has an antibiotic resistance mutation.

Embodiment 36. A polynucleotide, the polynucleotide encoding the rRNA of the engineered ribosome of any of embodiments 1-35.

Embodiment 37. The polynucleotide of embodiment 36, wherein the polynucleotide is a vector.

Embodiment 38. The polynucleotide of embodiment 36 or 37, wherein the polynucleotide further comprises a gene to be expressed by the engineered ribosome.

Embodiment 39. The polynucleotide of embodiment 38, wherein the gene is a reporter gene.

Embodiment 40. The polynucleotide of embodiment 39, wherein the reporter gene is a green fluorescent protein gene.

Embodiment 41. The polynucleotide of any of embodiments 36-40, wherein the engineered ribosome comprises a modified anti-Shine-Dalgarno sequence and the gene comprises a complementary Shine-Dalgarno sequence to the engineered ribosome.

Embodiment 42. The polynucleotide of any of embodiments 36-41, wherein the gene comprises a codon and the codon encodes for an unnatural amino acid.

Embodiment 43. A method for preparing an engineered ribosome, the method comprising expressing the polynucleotide of any of embodiments 36-42.

Embodiment 44. The method of embodiment 43, the method further comprising selecting a mutant.

Embodiment 45. The method of embodiment 44, wherein the selection step comprises a negative selection step, a positive selection step, or both a negative and a positive selection step.

Embodiment 46. An engineered cell, the engineered cell comprising (i) the polynucleotide of any of embodiments 36-42, (ii) the engineered ribosome of any of embodiments 1-35, or both (i) and (ii).

Embodiment 47. A engineered cell, the engineered cell comprising a first protein translation mechanism and a second protein translation mechanism, a. wherein the first protein translation mechanism comprises a ribosome, wherein the ribosome lacks a linking moiety between the large subunit and the small subunit and b. wherein the second protein translation mechanism comprises the engineered ribosome of any of embodiments 1-35.

Embodiment 48. A method for preparing a sequence-defined polymer, the method comprising (a) providing the engineered ribosome of any of embodiments 1-35 and (b) providing an mRNA or DNA template encoding the sequence-defined polymer.

Embodiment 49. The method of embodiment 48, wherein the sequence-defined polymer is prepared in vitro.

Embodiment 50. The method of embodiment 49, the method further comprising providing a ribosome-depleted cellular extract or purified translation system.

Embodiment 51. The method of embodiment 50, wherein the ribosome-depleted cellular extract comprises an S150 extract prepared from mid- to late-exponential growth phase cell cultures or cultures having an O.D.600˜3.0 at time of harvest.

Embodiment 52. The method of embodiment 48, wherein the sequence defined polymer is prepared in vivo.

Embodiment 53. The method of embodiment 48 or 52, wherein the sequence defined polymer is prepared in the cell of any of embodiments 46 or 47.

Embodiment 54. The method of any of embodiments 48-53, wherein the mRNA or DNA encodes a modified Shine-Dalgarno sequence and the engineered ribosome comprises an anti-Shine-Dalgarno sequence complementary to the modified Shine-Dalgarno sequence.

Embodiment 55. The method of any of embodiments 48-54, wherein the sequence-defined polymer comprises an amino acid.

Embodiment 56. The method of embodiment 55, wherein the amino acid is a natural amino acid.

Embodiment 57. The method of embodiment 55, wherein the amino acid is an unnatural amino acid.

Embodiment 58. The engineered cell of embodiment 47, wherein the ribosomes of the first protein translation mechanism comprise a modified anti-Shine-Dalgarno sequence, and wherein the ribosomes of the second protein translation system comprise an unmodified (e.g., wild-type) anti-Shine-Dalgarno sequence.

Embodiment 59. The method of any one of embodiments 48-53, further comprising untethered ribosomes comprising a modified anti-Shine-Dalgarno sequence.

Embodiment 60. The method of embodiment 59, wherein the mRNA or DNA encodes a modified Shine-Dalgarno sequence and the untethered ribosomes comprise an anti-Shine-Dalgarno sequence complementary to the modified Shine-Dalgarno sequence.

Embodiment 61. The method of embodiment of 60, wherein the sequence defined polymer comprises a natural or an unnatural amino acid.

Embodiment 62. An engineered cell comprising two or more protein translation mechanisms, wherein: (a) a first mechanism is the natural translation mechanism wherein mRNA is translated by a tethered, or stapled, ribosome in accordance with the natural genetic code; (b) a second mechanism is an artificial mechanism derived from a dissociable ribosome that tunes host metabolic burden or in which orthogonal mRNA comprising orthogonal codons is translated by this orthogonal ribosome.

Embodiment 63. An engineered cell comprising two or more protein translation mechanisms, wherein: (a) a first mechanism is the natural translation mechanism wherein mRNA is translated by a tethered, or stapled, ribosome that sustains the life of the cell; (b) a second mechanism is an artificial mechanism derived from a dissociable ribosome that carries out an orthogonal function.

Embodiment 64. An engineered cell comprising two or more protein translation mechanisms, wherein the orthogonal dissociable ribosomes outperforms an orthogonal tethered ribosomes in the context of protein expression.

Embodiment 65. An engineered cell in which not only the o-30S, but also the free 50S subunit is engineered to achieve new functionalities.

Embodiment 66. An engineered cell in which not only the o-30S, but also the free 50S subunit are engineered to achieve new functionalities without interfering with the expression of the cellular proteome not only is the o-30S, but also the free 50S subunit is engineered to achieve new functionalities without interfering with the expression of the cellular proteome.

Embodiment 67. An engineered cell in which not only the o-30S, but also the free 50S subunit is engineered to achieve gain of function ribosome mutations.

Embodiment 68. An engineered cell in which not only the o-30S, but also the free 50S subunit is engineered to achieve gain of function ribosome mutations, wherein these mutations specifically overcome the translation of problematic polymer sequences.

Embodiment 69. An engineered cell comprising a first protein translation mechanism and a second protein translation mechanism, the first protein translation mechanism comprising a first engineered ribosome, the first engineered ribosome comprising: i) a small subunit comprising ribosomal RNA (rRNA) and protein, ii) a large subunit comprising ribosomal RNA (rRNA) and protein, and iii) a linking moiety, wherein the linking moiety comprises a polynucleotide sequence and tethers the rRNA of the small subunit with the rRNA of the large subunit; the second protein translation mechanism comprising a second engineered ribosome, the second engineered ribosome comprising: i) a small subunit comprising rRNA and protein, ii) a large subunit comprising rRNA and protein, and iii) wherein the second engineered ribosome lacks a linking moiety between the large subunit and the small subunit; and wherein small subunit of the second engineered ribosome comprises a modified anti-Shine-Dalgarno sequence to permit translation of templates having complementary and/or cognate Shine-Dalgarno sequence different from endogenous cellular mRNAs, and/or wherein the second engineered ribosome comprises one or more change-of-function mutations, wherein the change-of-function mutation is not at the anti-Shine Dalgarno sequence.

Embodiment 70. The engineered cell of embodiment 69, wherein the first and the second protein translation mechanisms are capable of supporting translation of a sequence defined polymer.

Embodiment 71. The engineered cell of any one embodiments 69-70, wherein the first protein translation mechanism is capable of supporting translation of native, endogenous RNAs.

Embodiment 72. The engineered cell of any one embodiments 69-71, wherein the second protein translation mechanism is capable of supporting translation of non-native, exogenous RNAs.

Embodiment 73. The engineered cell of any one embodiments 69-72, wherein the small subunit of the second engineered ribosome comprises a modified anti-Shine-Dalgarno sequence selected from the group consisting of 3′-GGUGUU-5′, 3′-UGGUGU-5′, 3′-GGUGUC-5′, 3′-GUUUAG-5′, 3′-UGGAAU-5′, 3′-GGAUCU-5′, 3′-UGGAUC-5′, 3′-UGGUAA-5′, and 3′-UGGAUC-5′.

Embodiment 74. The engineered cell of any one embodiments 69-74, wherein the second engineered ribosome comprises a change-of-function mutation in one or more of: a) peptidyl transferase center (PTC); b) nascent peptide exit tunnel (NPET); c) interaction site with elongation factors; d) tRNA binding sites; e) chaperone binding sites; f) nascent chain modifying enzyme biding sites; g) GTPase center.

Embodiment 75. The engineered cell of any one embodiments 69-74 wherein the large subunit of the second engineered ribosome comprises a change-of-function mutations at one or more of the following residues of a 23S rRNA: G2061, C2452, U2585, G2251, G2252, A2057, A2058, C2611, A2062, A2503, U2609, G2454, and G2455.

Embodiment 76. The engineered cell of any one embodiments 69-75, wherein the first, the second, or both the first and the second engineered ribosomes comprises an antibiotic resistance mutation.

Embodiment 77. The engineered cell of any one embodiments 69-76, wherein the large subunit of the first engineered ribosome comprises a permuted variant or mutant of a 23SrRNA and/or the small subunit comprises a permuted variant or mutant of a 16S rRNA.

Embodiment 78. The engineered cell of any one embodiments 69-77, wherein the linking moiety covalently bonds a helix of the large subunit selected from the group consisting of helix 10, helix, 38, helix 42, helix, 54, helix 58, helix, 63, helix 78, helix, 101, to a helix of the small subunit selected from the group consisting of helix 11, helix, 26, helix 33, and helix 44.

Embodiment 79. A method for preparing a sequence-defined amino acid polymer, the method comprising (a) providing one or more of: (i) the cell of any one of embodiments 69-78; (ii) a cell extract derived from the cell of any one of embodiments 69-78; (iii) purified translation system derived from the cell of any one of embodiments 69-78; b) providing an mRNA encoding the sequence-defined polymer to the cell or the cell extract.

Embodiment 80. The method of embodiment 79, wherein the sequence-defined amino acid polymer is prepared in vivo.

Embodiment 81. The method of embodiment 79, wherein the sequence-defined amino acid polymer is prepared in vitro.

Embodiment 82. The method of any one of embodiments 79-81, wherein the sequence-defined amino acid polymer comprises one or more unnatural amino acids.

### EXAMPLES

The following Examples are illustrative and should not be interpreted to limit the claimed subject matter.

### Example 1. Development and Testing of a Fully Orthogonal System for Protein Synthesis in Bacterial Cells

A. Abstract

The ribosome synthesizes genetically-encoded polypeptides from proteinogenic amino acids. Ribosome engineering is emerging as a powerful approach for expanding the catalytic potential of the protein synthesis apparatus and for elucidating its origin, evolution and function. Because the properties of the engineered ribosome might be detrimental for the general protein synthesis, the designer ribosome needs to be functionally isolated from the translation machinery synthesizing cellular proteins. The initial solution to this problem has been offered by Ribo-T, an engineered ribosome with the tethered subunits which, while translating a desired protein, could be excluded from translation of the cellular proteome. Here, we provide a conceptually different design of an engineered cell with two orthogonal translation systems, whereby the cellular proteins are translated by Ribo-T, while the native ribosome operates as a segregated protein synthesis machine with its both subunits committed to the translation of specific kind of mRNA. We show that both subunits of the specialized ribosome retain autonomy from Ribo-T, excluded from translation of the cellular proteome and thus, could be engineered for new functions. We illustrate the utility of this system by generating a comprehensive collection of mutants with variations at every rRNA nucleotide of the peptidyl transferase center and isolating gain-of-function mutations that enable the ribosome to overcome the translation termination blockage imposed by the arrest peptide.

B. Introduction

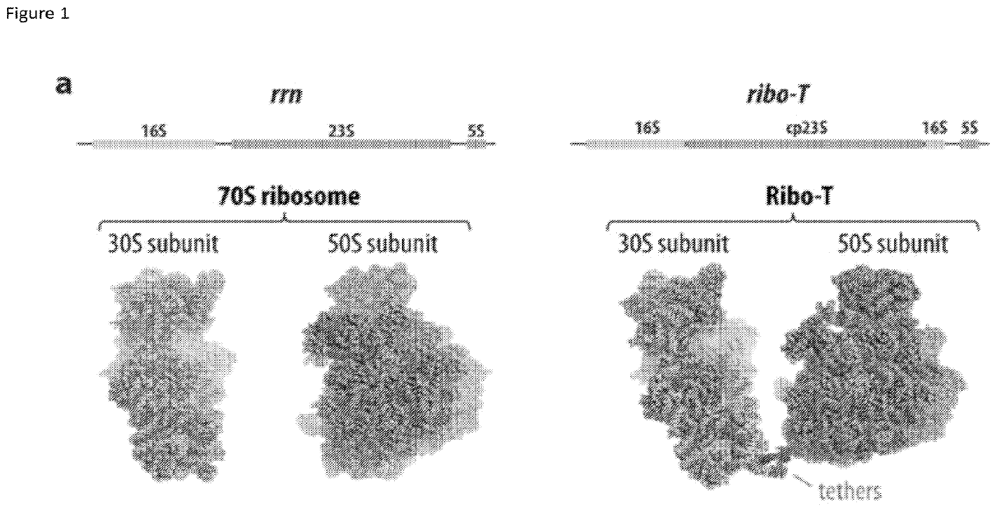

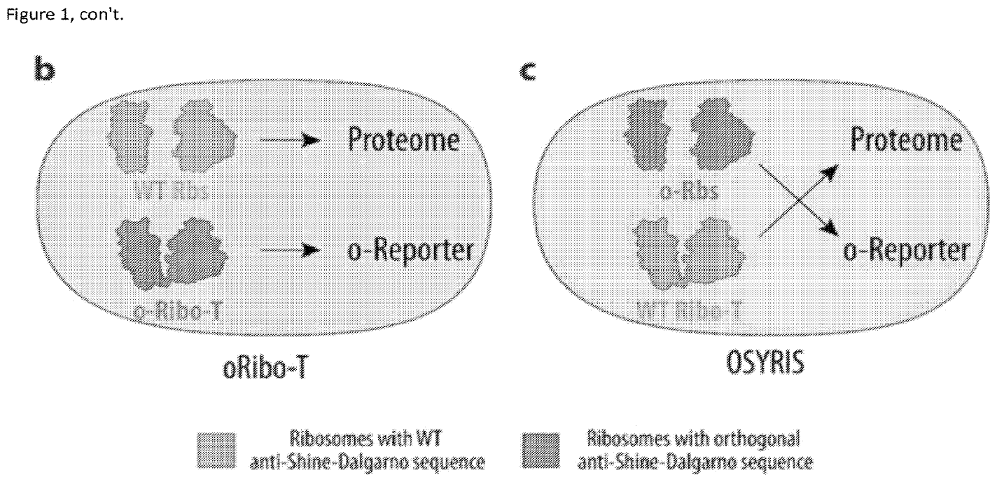

The ribosome performs distinct, complex, and highly coordinated functions during protein synthesis. It is composed of two subunits, small and large, which in bacteria are the 30S and 50S, respectively (FIG. 1a). The 30S subunit drives the initiation of translation using the complementarity between the Shine-Dalgarno sequence (SD) in the vicinity of the mRNA's start codon and the anti-Shine-Dalgarno sequence (ASD) at the 3′ end of its 16S rRNA¹. At the elongation stage of protein synthesis, the 30S subunit carries out the decoding function by sustaining codon-anticodon interactions, while at termination it facilitates the recognition of the stop codons by the release factors. The 50S subunit hosts the peptidyl transferase center (PTC) where polymerization of amino acids into a polypeptide takes place and also, at the termination phase, peptide release is catalyzed. The growing amino acid chain advances from the PTC into the nascent peptide exit tunnel (NPET) on its way out from the ribosome^{2,3}.

The ribosome has evolved to operate with its natural substrates (mRNAs, tRNAs, and proteinogenic amino acids) enabling it to synthesize genetically-encoded proteins. Nevertheless, its synthetic capabilities could be expanded by molecular engineering to allow the use of alternative genetic codes, polymerization of a wider variety of amino acids, or even carry out a programmable synthesis of non-proteinaceous polymers⁴. Ribosome engineering could be also employed for elucidating the origin, evolution and function of the protein synthesis apparatus. All such endeavors, however, require altering the intrinsic properties of the ribosome⁵ that inevitably diminish or even abolish the ribosome's ability to synthesize cellular proteins^{6,7}. Although interesting solutions to this problem could be offered by cell-free translation systems⁸, the efficiency and scalability issues limit their current application.

The ribosome engineering predicament can be overcome by creating an orthogonal protein synthesis apparatus within the cell that does not participate in the production of the cellular proteome and is exclusively dedicated to the translation of only one or several specific mRNAs⁹. By mutating the ASD in the 16S rRNA and introducing a complementary SD sequence into an mRNA, it has been possible to direct a fraction of small subunits to the translation of only the cognate mRNA^{10,11}, a strategy that has been exploited for expanding the decoding capacity of the ribosome¹². The orthogonality of this set-up, however, is limited to only the small subunit because, due to the stochastic nature of the association of large and small ribosomal subunits in multiple rounds of translation, both the wild-type (wt) and the orthogonal 30S subunits share the same pool of 50S subunits. The inability to create orthogonal 50S subunits has limited the efforts to remodel the PTC and the NPET, the most critical sites for designing a translation apparatus with modified or expanded catalytic capacity. The engineering of the first fully orthogonal translation system became possible with the advent of the ribosome with tethered subunits, Ribo-T¹³. In Ribo-T, and in subsequent similar designs¹⁴⁻¹⁶, circularly-permutated 23S rRNA is embedded into the 16S rRNA, yielding a ribosome whose subunits are tethered by two RNA linkers (FIG. 1a). Because small and large subunits of Ribo-T are inseparable, in the orthogonal Ribo-T (oRibo-T) with altered ASD both subunits are committed to translating exclusively the cognate mRNA and thus, oRibo-T functions independently from the wt ribosomes that translate the cellular proteins (FIG. 1b). With the help of oRibo-T it was possible to select specific PTC mutations that facilitate the polymerization of the amino acid sequences problematic for the wt ribosome^{13,15}. However, while achieving full orthogonality, the unusual design of Ribo-T limits its functionality. Ribo-T translates proteins with only half the rate of the dissociable ribosome¹³. It is slower in departing from the start codons in comparison with the wt ribosomes¹⁷. Furthermore, the biogenesis of even ‘wt’ Ribo-T is rather slow and inefficient¹⁷ and the assembly problems could be additionally exacerbated if the ribosome's functional centers are subjected to additional alterations⁷. While not characterized as extensively, we anticipate similar challenges with “stapled” ribosomes. Taken together, all these factors complicate the direct use of Ribo-T, or any tethered ribosome, in further engineering efforts.

C. Development and Testing of a “Flipped” Orthogonal System for Protein Synthesis in Bacterial Cells

In order to overcome the shortcomings of the original oRibo-T-based approach for engineering cells with two functionally-independent translation machineries, we have now created a conceptually new design of an in vivo system that utilizes dissociable, yet fully segregated, ribosomes dedicated to translation of only specialized mRNAs. By ‘flipping’ the roles of Ribo-T and dissociable ribosomes, we engineered bacterial cells where translation of the proteome is carried out by Ribo-T, whereas the ribosome, composed of the dissociable orthogonal 30S (o-30S) subunit and wt 50S subunit functions as a fully orthogonal translation machine (FIG. 1c). In the resulting setup, that we named OSYRIS (Orthogonal translation SYstem based on Ribosomes with Isolated Subunits), complete orthogonality is achieved because the tethered nature of Ribo-T precludes it from associating with either the o-30S or the 50S of the dissociable ribosome. Therefore, in OSYRIS cells, the physically-unlinked o-30S and 50S ribosomal subunits are nevertheless compelled to interact with each other and function as fully orthogonal ribosomes (o-ribosomes). As a result, not only the o-30S, but also the free 50S subunit can be engineered to achieve new functionalities without interfering with the expression of the cellular proteome (FIG. 1c).

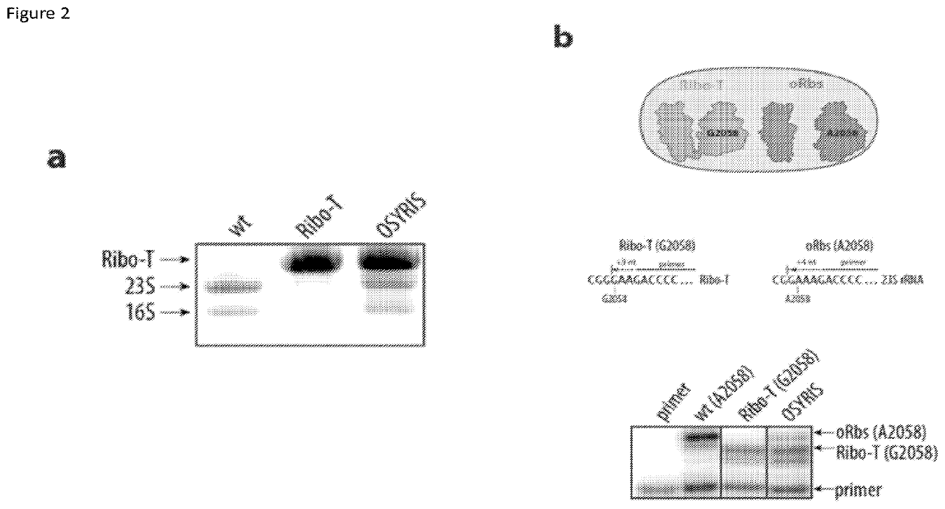

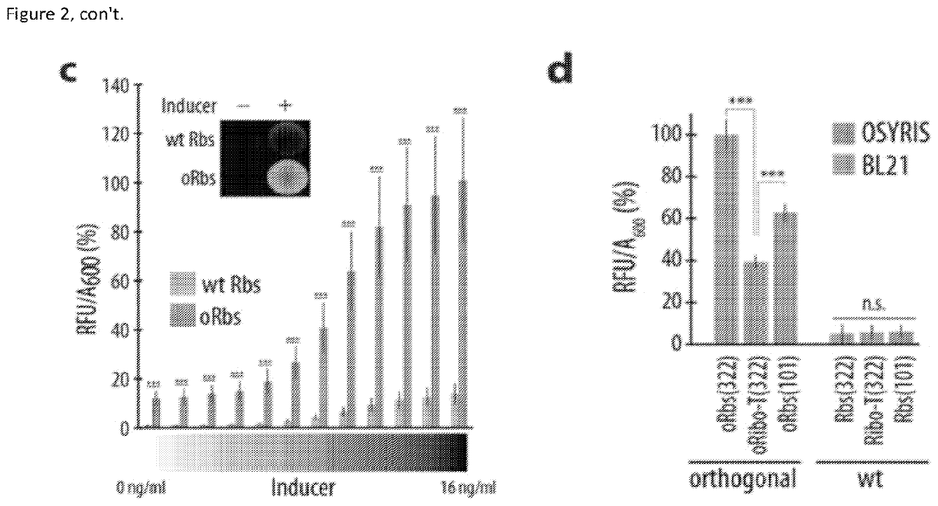

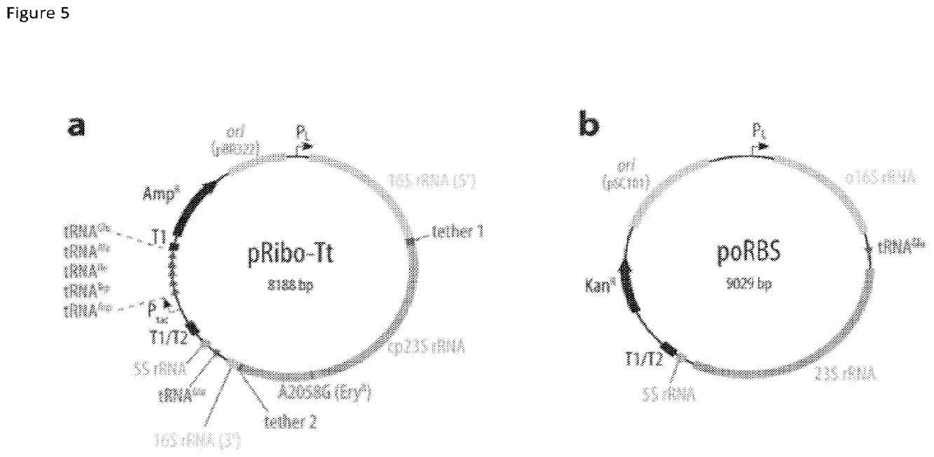

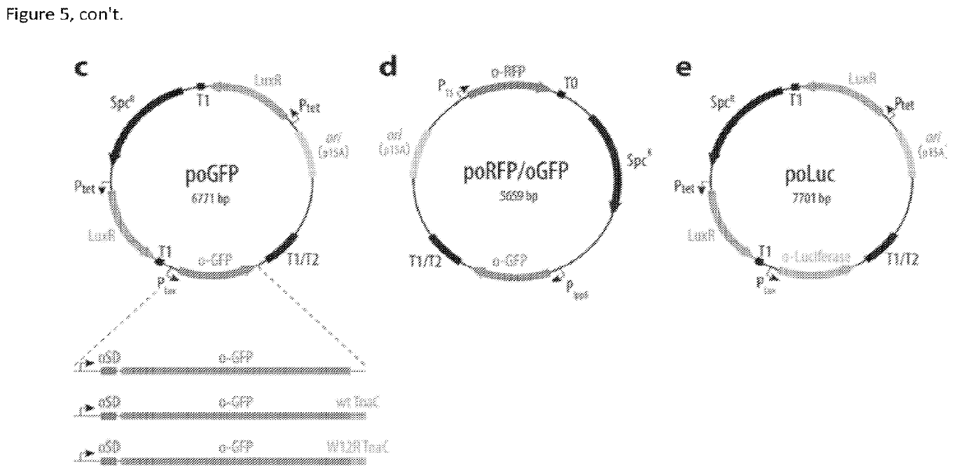

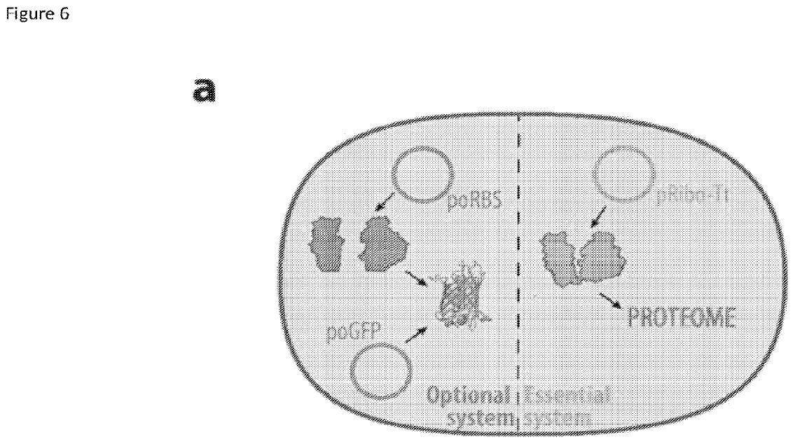

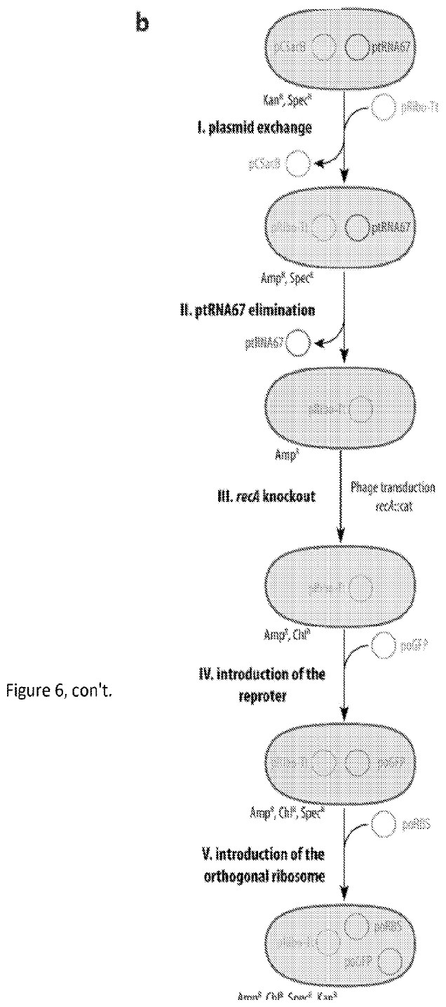

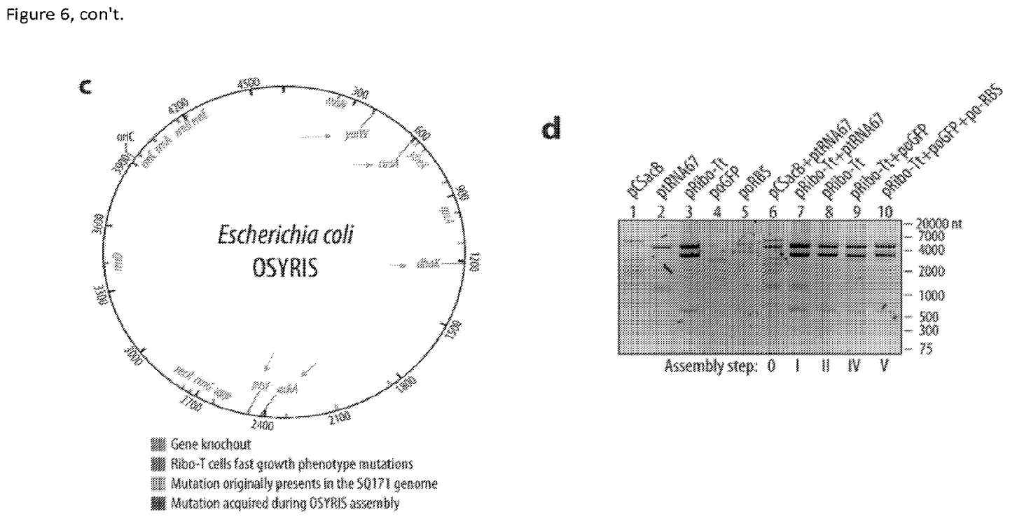

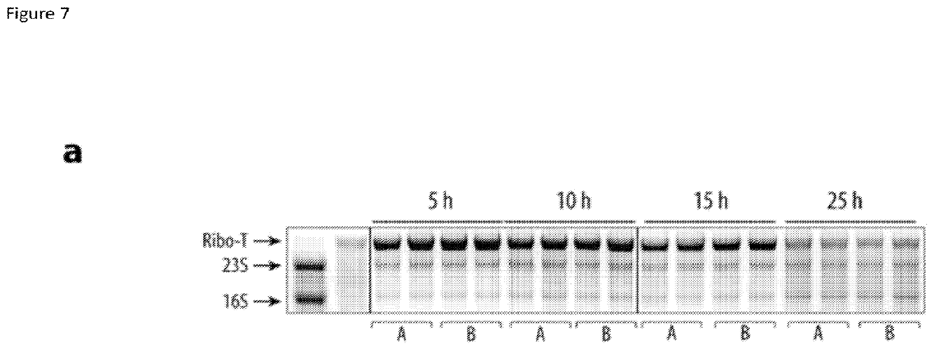

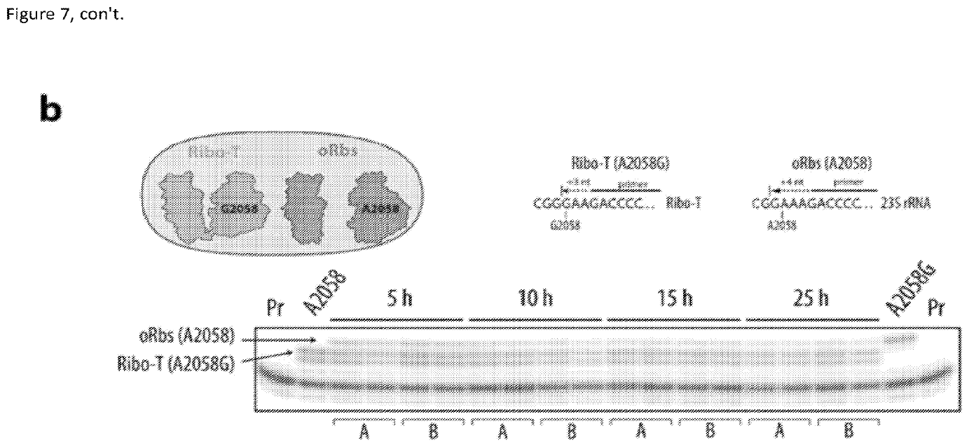

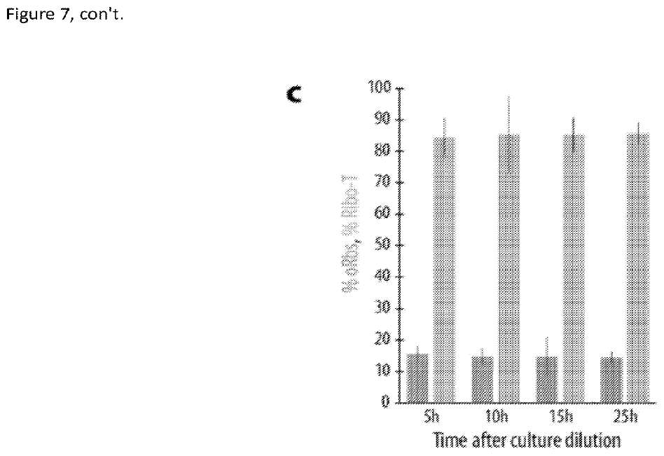

The components of the system (FIG. 5) were assembled in an E. coli strain that lacks chromosomal rrn alleles¹⁸ (FIG. 6). In the resulting OSYRIS cells, the Ribo-T rRNA with improved 16S-23S tethers¹⁶ is expressed from the optimized pRibo-Tt plasmid. Another plasmid, poRbs, carries the rRNA genes of the dissociable o-ribosomes, whose 16S rRNA gene carry an altered ASD (FIG. 5). In the cells transformed with these two plasmids, the o-ribosomes account for ˜15% of the total ribosomal population (FIG. 2a,b and FIG. 7). Specialized reporter genes (gfp, rfp or luc) with an SD cognate to that of the o-ribosome ASD are introduced on a third plasmid (poGFP, poRFP/oGFP or poLuc) (FIG. 5) (we will refer to these orthogonal reporters as o-reporters).

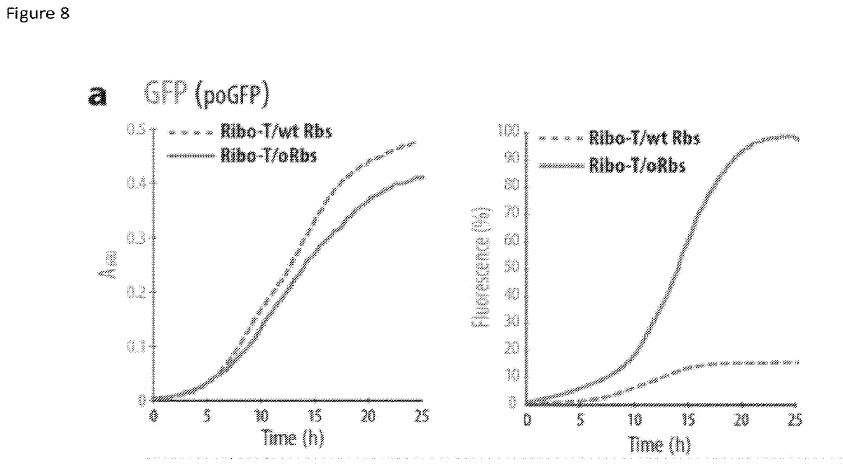

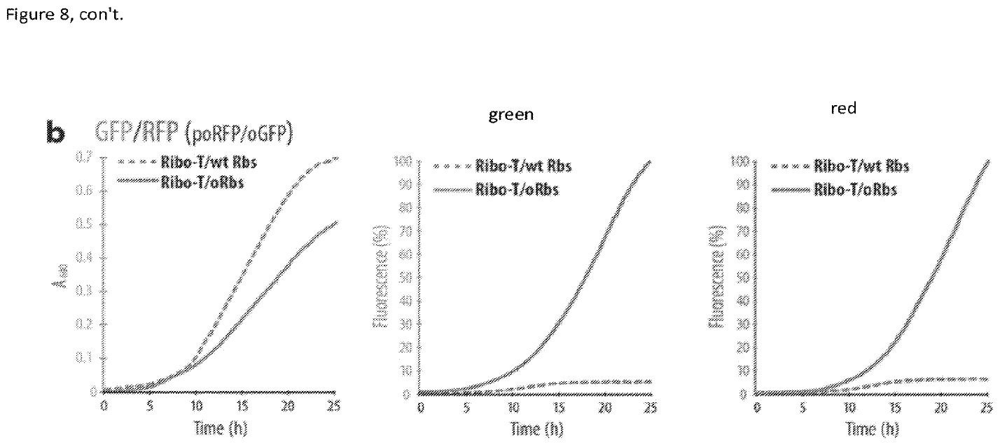

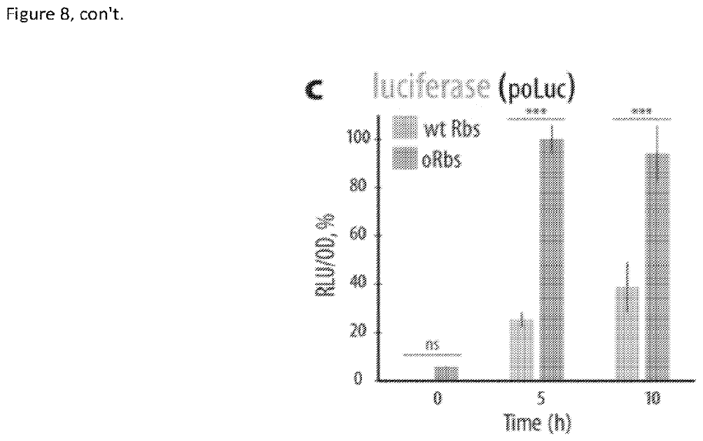

The expression of the o-reporters in the OSYRIS cells relies on the o-ribosomes: in their absence, the reporter proteins (GFP, RFP or luciferase) encoded by the o-mRNAs are produced at low levels, whereas the presence of the dissociable o-ribosomes, greatly stimulates the o-reporter expression (FIG. 2c,d and FIG. 8). Thus, o-30S subunits, whose exclusion from translation of cellular mRNAs has been confirmed in previous studies^{11,16}, efficiently drive translation of o-mRNAs in OSYRIS. Of note, dissociable o-ribosomes outperformed oRibo-T in expression of the o-reporters when introduced in the same host (E. coli, BL21) on the comparable vectors Furthermore, relative expression of the o-GFP reporter in the OSYRIS cells, where o-ribosomes are expressed from a low-copy number plasmid, is higher in comparison with cells expressing oRibo-T from a higher-copy number plasmid (FIG. 2d, FIG. 8, dark bars).

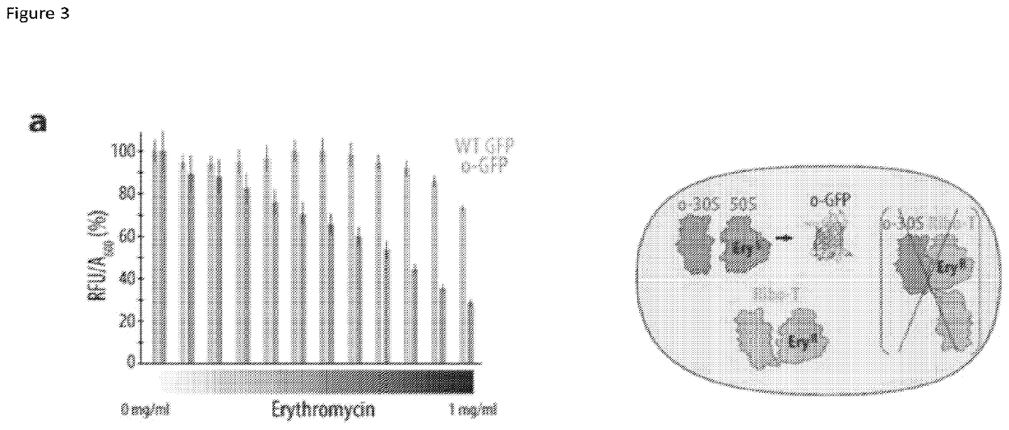

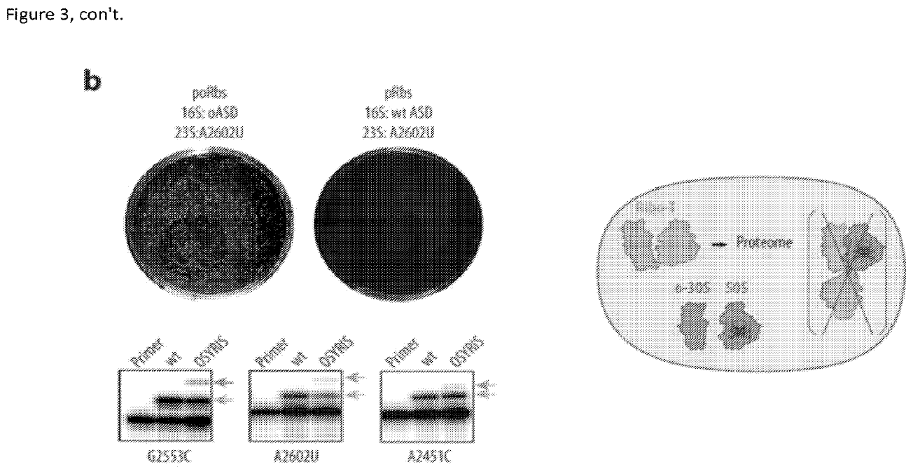

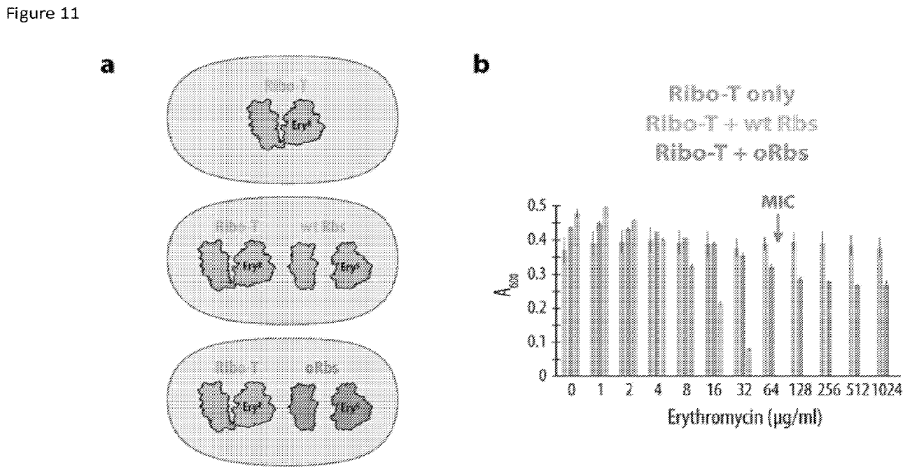

To test whether only the small subunits or both the small and large subunits of the dissociable o-ribosomes in the OSYRIS cells remain functionally isolated from Ribo-T, we took advantage of the A2058G mutation present in Ribo-T that renders it resistant to the antibiotic erythromycin (Ery)¹³. If the free 50S subunits, which are Ery-sensitive, could somehow cooperate with the small subunits of Ribo-T in translating the proteome, Ery would inhibit general protein synthesis and interfere with cell growth. However, OSYRIS cells continue to grow even at the highest tested concentration of the antibiotic (1 mg/ml), demonstrating the functional autonomy of the dissociable 50S subunit and Ribo-T (FIG. 11, second set of bars in each group). In contrast, expression of the o-GFP reporter progressively decreased with the increase of Ery concentration in the medium (FIG. 3a). This result indicates that translation of the o-reporter is driven primarily by the ribosome composed of dissociable o-30S and 50S subunits, as opposed to o-30S/Ribo-T hybrids (FIG. 3a). Thus neither o-30S subunit, nor 50S subunit interact with Ribo-T and both subunits remain functionally dedicated to each other in spite of the lack of a physical linkage between them.

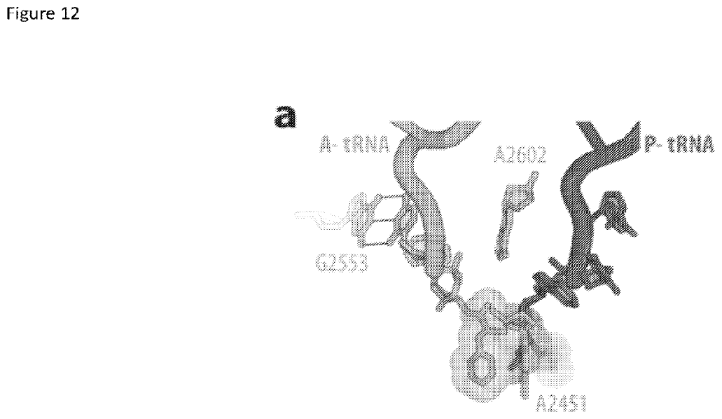

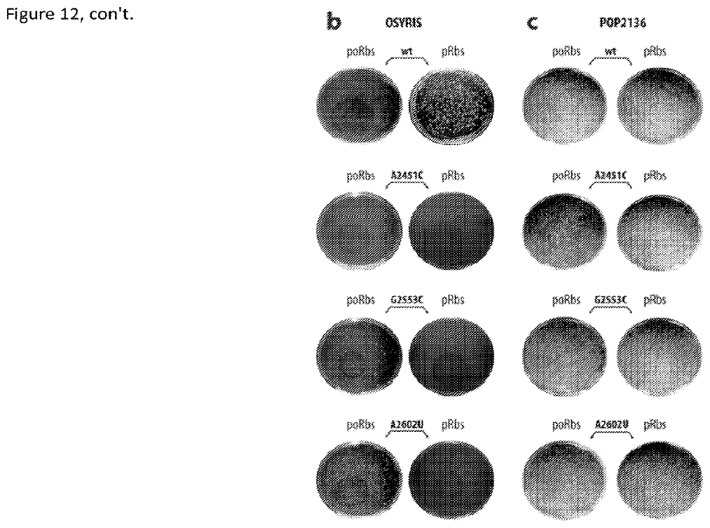

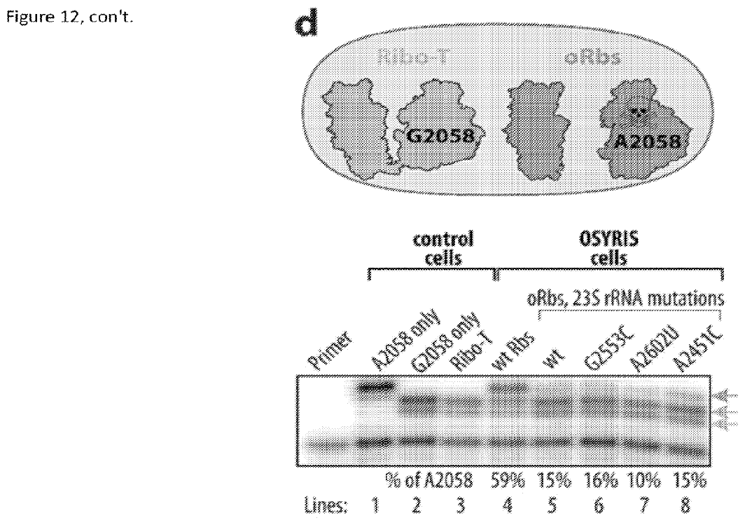

A more rigorous proof for the orthogonality of the dissociable 50S subunits in the OSYRIS cells was obtained by introducing mutations into its 23S rRNA that are known to be dominantly lethal in wt E. coli cells^{20,21}. Two of these mutations, A2451C and A2602U, alter critical nucleotides of the PTC active site, while mutation G2553C disrupts essential rRNA-tRNA interactions required for the proper placement of the A-site aminoacyl-tRNA for peptide bond formation^{22,23} (FIG. 12a). If the mutant dissociable 50S subunits interact primarily with the o-30S subunits, survival of the OSYRIS cells should not be compromised because o-ribosome is excluded from general translation. If, on the contrary, the free 50S subunits associate with Ribo-T and participate in translation of the proteome, the dominantly lethal 23S rRNA mutations would prevent or severely compromise the growth of the OSYRIS cells. Attempts to express the mutant 50S subunits in the cells lacking o-30S (by transforming Ribo-T cells with the pRbs plasmid encoding the mutant 23S rRNAs along with wt 16S rRNA) yielded no transformants, confirming the dominantly lethal nature of the 23S rRNA mutations (FIG. 3b and FIG. 12b,c). In contrast, when the mutant 23S rRNA gene was introduced in OSYRIS cells on the plasmid carrying orthogonal 16S rRNA, many transformants appeared (FIG. 3b and FIG. 12b,c). Analysis of the rRNA isolated from the cultures of the transformed cells revealed fairly high expression level of the free 50S subunits containing the mutant 23S rRNA (FIG. 3b, FIG. 12d). Altogether, these results clearly demonstrate that the dissociable large ribosomal subunit remains functionally isolated from Ribo-T. These results clearly demonstrate that the dissociable large ribosomal subunit remains functionally isolated from Ribo-T.

Altogether, the results of the o-reporter expression and tolerance to dominantly lethal mutations show that in the OSYRIS cells, the dissociable o-ribosomes translate o-mRNAs but do not significantly contribute to translation of the proteome. Therefore, both subunits of the dissociable o-ribosomes in OSYRIS cells are suitable for biomolecular engineering.

Having established the orthogonality of the dissociable ribosome in the OSYRIS cells, we carried out a proof-of-principle experiment to test the potential of the system for selecting mutations in the rRNA of the large subunit that would enable the ribosome to carry out otherwise problematic tasks. Specifically, we aimed to engineer a ribosome capable of efficient release of difficult-to-terminate proteins. In general, release of a fully synthesized polypeptide is a highly-nuanced reaction catalyzed by the PTC with the assistance of class 1 release factors^{24,25}. While most proteins are efficiently released at the stop codons, termination of others can be more problematic^{26,27}. An extreme case of inefficient termination in E. coli is represented by programmed translation arrest at the stop codon of the mRNA encoding the regulatory protein TnaC²⁸⁻³⁰. At high concentrations of tryptophan, the release of the fully-translated TnaC is inhibited and the resulting stalling of the ribosome at the tnaC stop codon leads to the activation of the expression of the downstream genes of the tna operon³¹. The termination arrest at the tnaC stop codon is mediated by unfavorable interactions of the nascent TnaC with rRNA nucleotides of the NPET and the PTC^{30,31}. The TnaC-mediated termination arrest represents a paradigm of inefficient protein release and illustrates one of the issues that could curb the expression of bioengineered polypeptides carrying, for example, non-canonical amino acids.

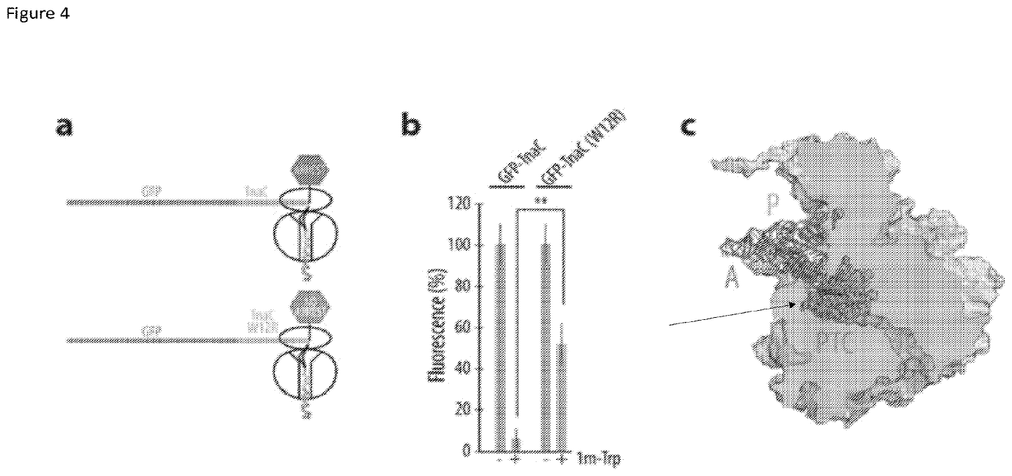

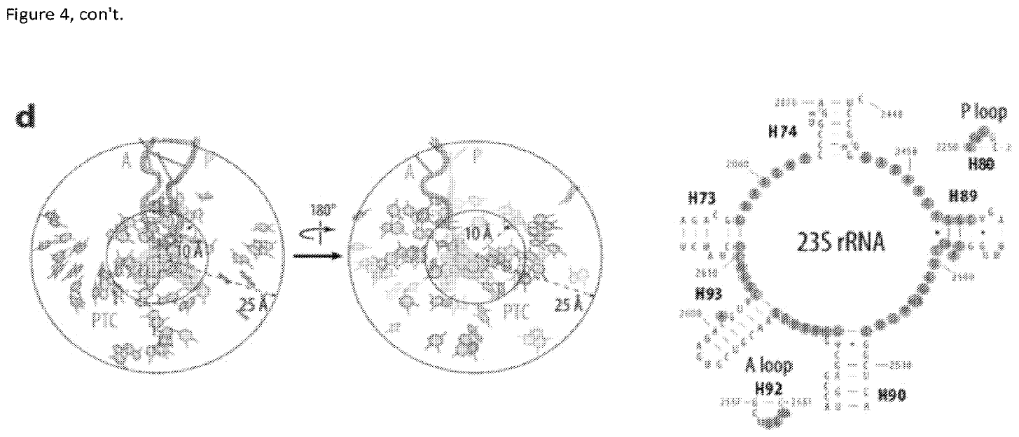

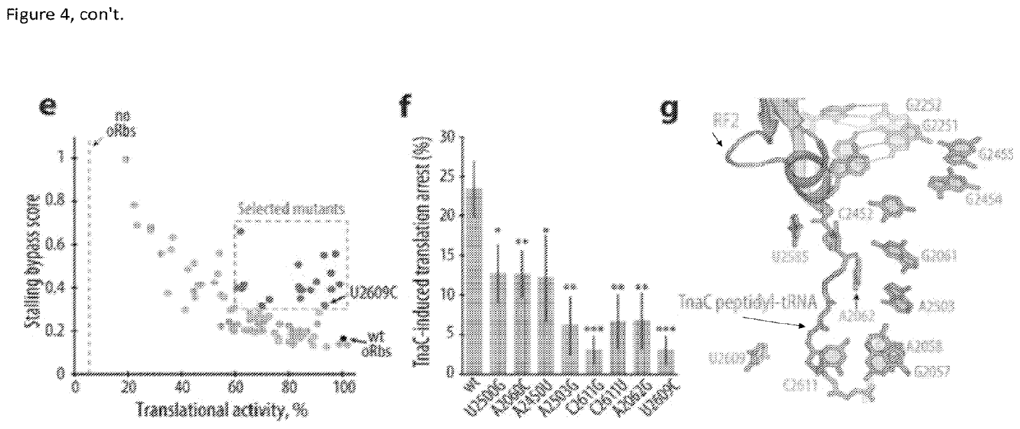

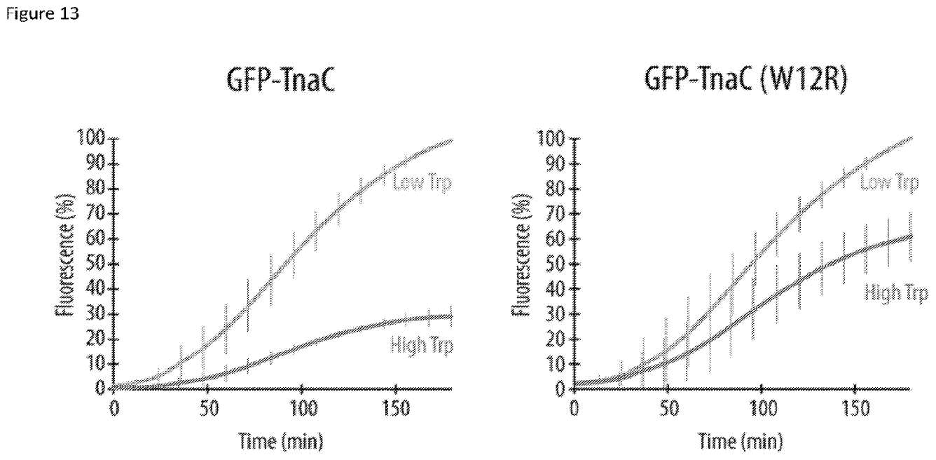

In order to identify mutations that could alleviate the inefficient termination of TnaC, we constructed a reporter in which the TnaC-coding sequence (lacking its own start codon) was appended at the end of the gfp gene (FIG. 4a). As expected, in vivo and in vitro expression of the GFP-TnaC chimera was inhibited at high concentration of tryptophan (FIG. 4b, FIG. 13). Introduction of the W12R mutation in the TnaC-coding segment, known to alleviate the termination arrest²⁸, significantly stimulated the reporter expression in the presence of tryptophan (FIG. 4b, FIG. 13).

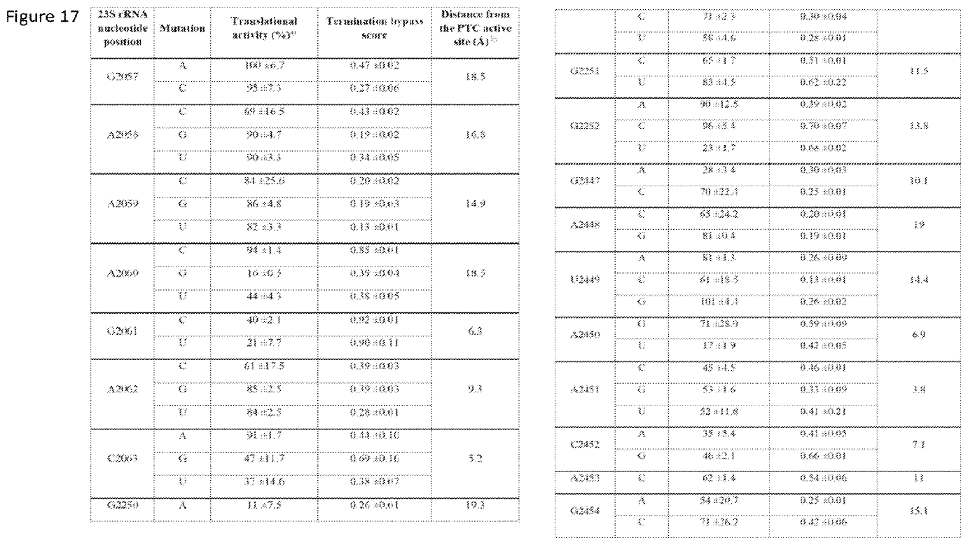

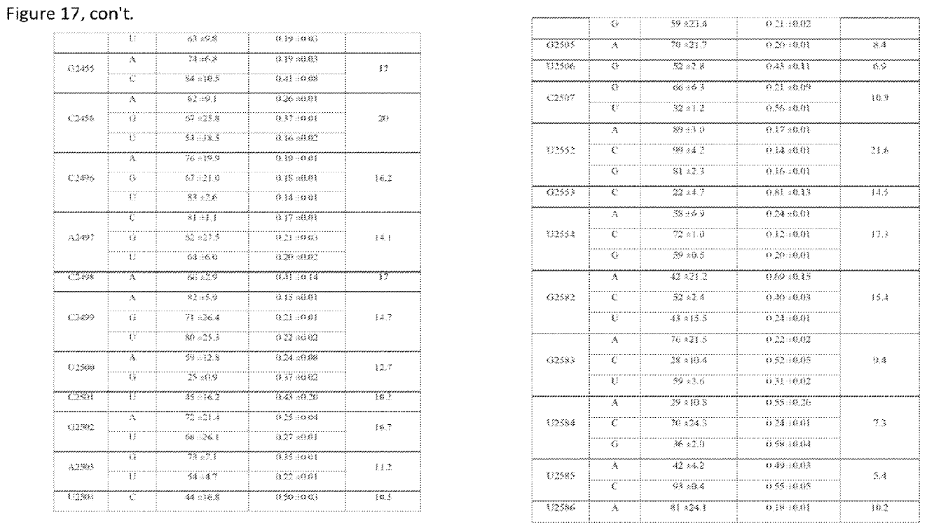

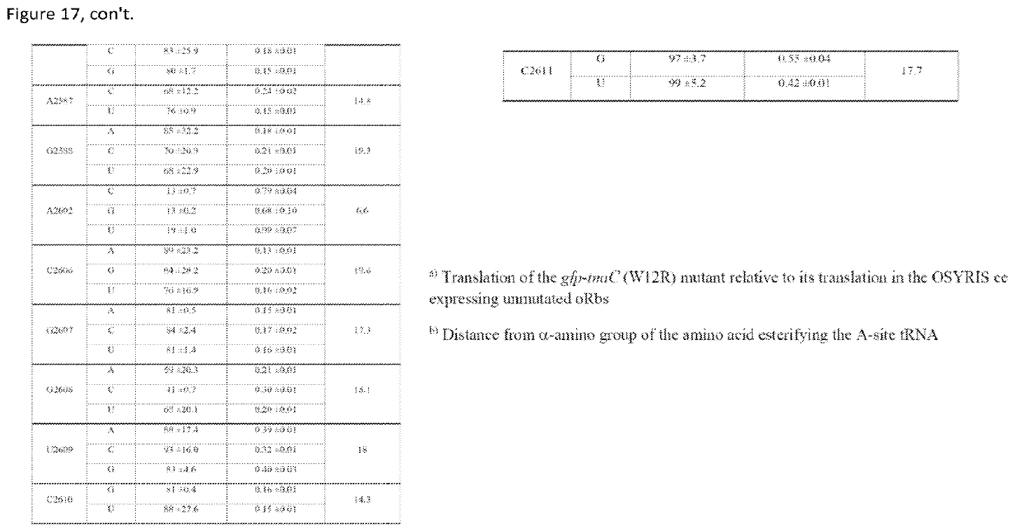

We then generated a comprehensive library of 120 single-nucleotide 23S rRNA mutants in the poRbs plasmid (FIG. 5, Table at FIG. 17) which included alterations at: i) every of the 9 rRNA residues in the PTC active site located within a 10 Å radius of the attacking amine of the acceptor amino acid participating in the peptidyl-transfer reaction; ii) 41 of the second-shell nucleotides (those within a 25 Å radius from the PTC); iii) 6 residues of the 23S rRNA P- and A-loops involved in positioning the acceptor ends of the P- and A-site tRNAs (FIG. 4c,d). Of note, most of the individual mutations included in the library carried by the OSYRIS cells have been reported to be deleterious or lethal in wt E. coli cells^{20,21}, and thus could be readily tested only due to the orthogonal nature of dissociable ribosomes in the OSYRIS cells.

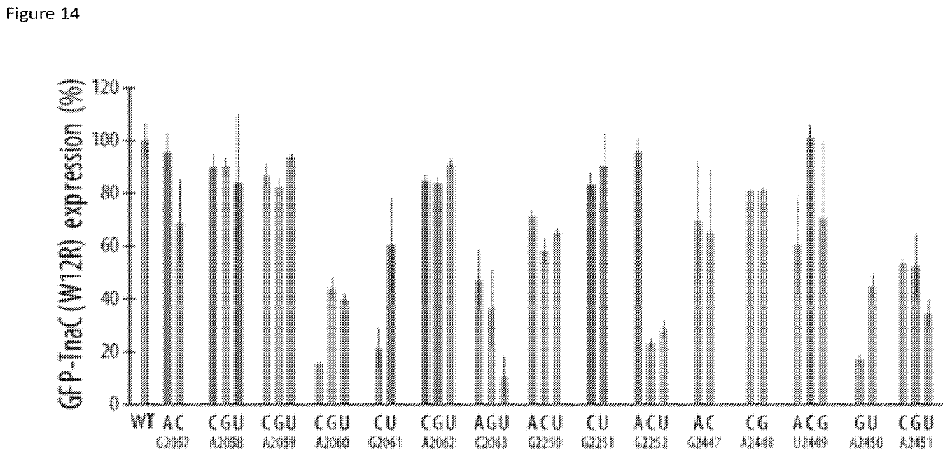

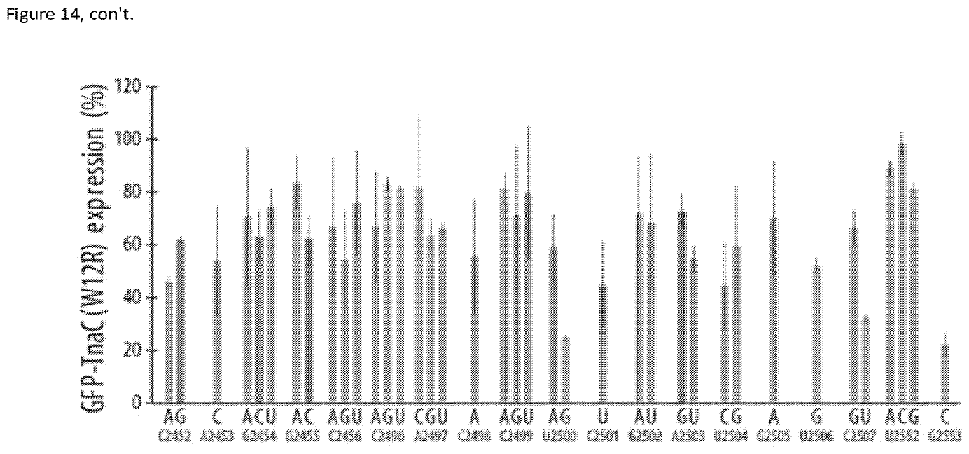

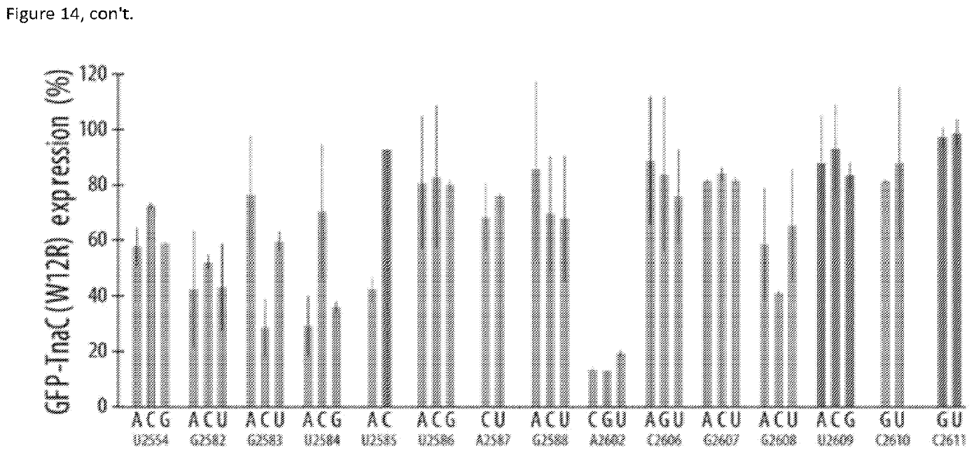

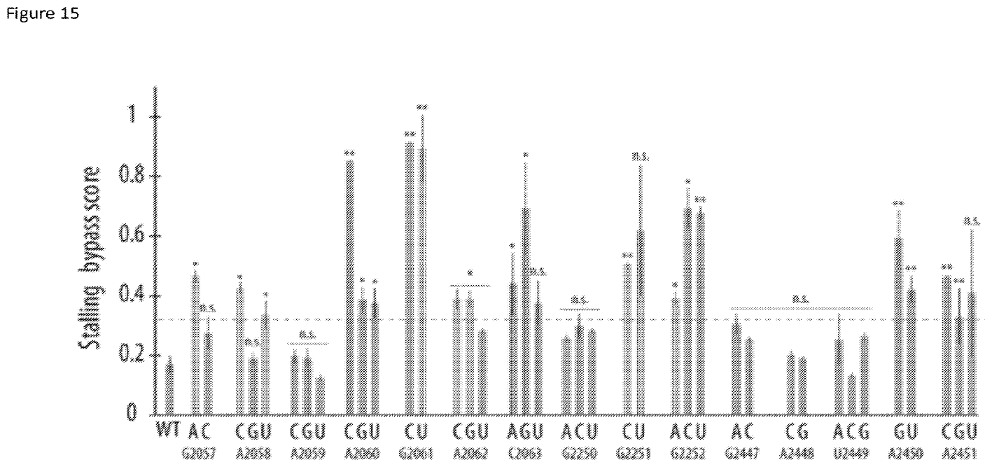

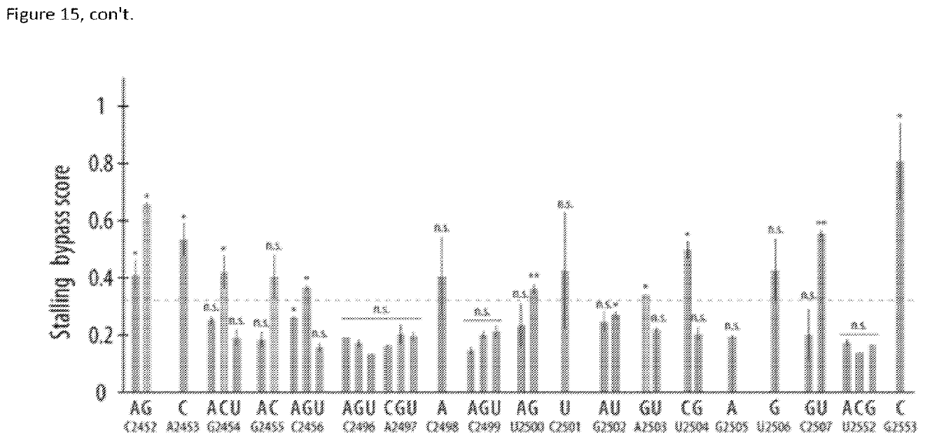

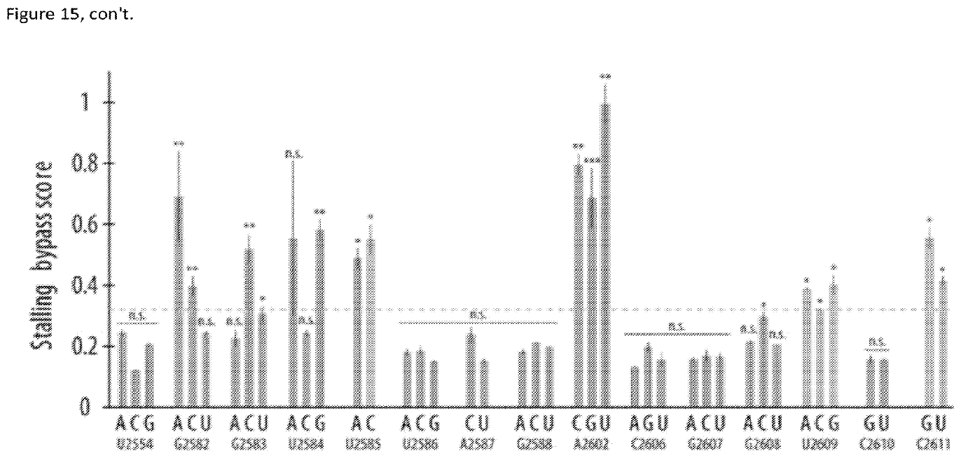

We characterized the ability of the individual mutants to successfully terminate the GFP-TnaC polypeptide by estimating the stalling bypass (SB) score. The SB score reflects the relative expression of the hard-to-terminate GFP-TnaC reporter in comparison with the GFP-TnaC(W12R) variant that terminates efficiently²⁸. In addition, the expression level of the GFP-TnaC(W12R) construct was used to evaluate the effect of the PTC mutations on the general translation activity of the mutant ribosome. Strikingly, a number of the mutants with alterations in the PTC rRNA residues exhibited a notably higher bypass score than the OSYRIS cells with wild type 50S subunit (FIG. 4e and FIGS. 14 and 15). Among these, 19 mutants combined high translation activity (>60% of the wt control) with a significantly increased SB score (>0.3 vs. 0.17 for the wt control) (FIG. 4e, Table at FIG. 17). The identified mutations were at the 23S rRNA residues located in the PTC active site (G2061, C2452, U2585), the P-loop (G2251, G2252) and in the second PTC shell, including residues at the NPET entrance (A2057, A2058, C2611, A2062, A2503, U2609) and two residues (G2454 and G2455) that via A2453 stack upon C2452 of the PTC (FIG. 4g). Two of the non-lethal mutations within this list (U2609C and A2058U) have been described previously^{29,31}; they served as an internal control confirming the the newly-isolated mutants indeed reveal the PTC residues involved in the TnaC-mediated termination arrest and that the identified mutations help to overcome ribosome stalling at the TnaC stop codon.

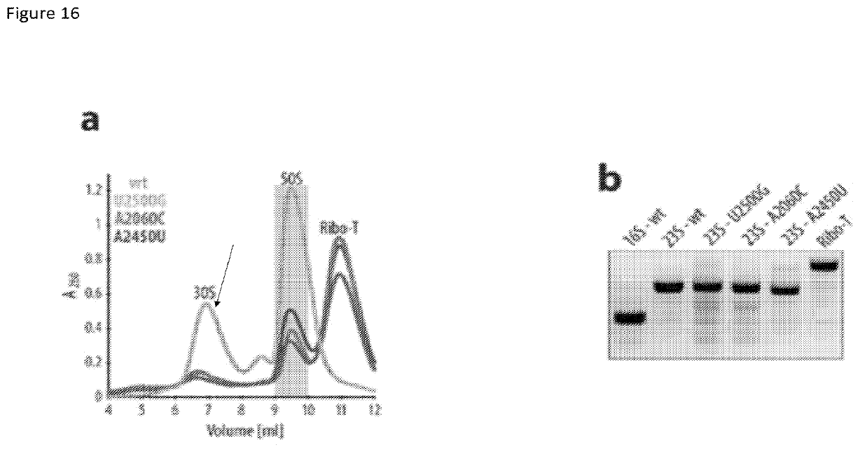

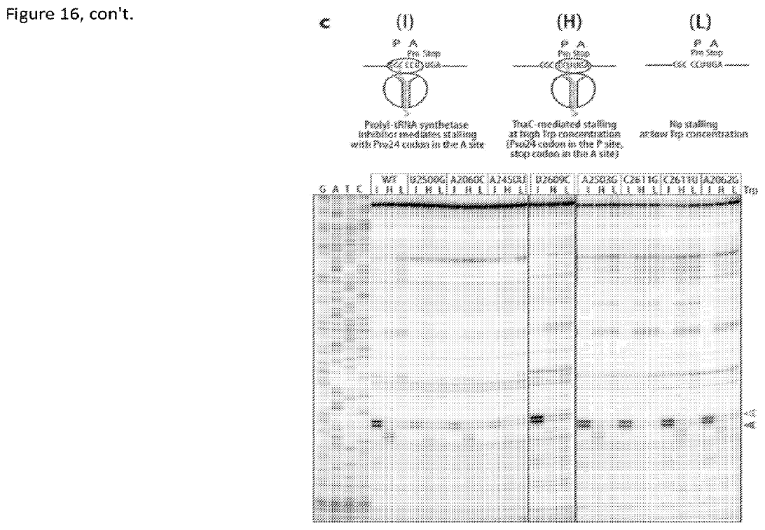

A unique opportunity offered by the OSYRIS cells is the possibility of isolating individual ribosomal subunits with even lethal mutations because dissociable 30S or 50S subunits can be separated from Ribo-T by sucrose gradient centrifugation¹³ (FIG. 16a,b). Taking advantage of this feature of the system, we prepared large ribosomal subunits carrying lethal mutations U2500G, A2060C, A2450U that showed bypass score>0.37, re-associated them with wt (non-orthogonal) 30S subunits, and tested in a cell-free translation system whether the mutations relieve ribosome stalling at the stop codon of the tnaC ORF (FIG. 16c). We also tested some of the non-lethal mutants (A2503G, A2062G, C2611G, C2611U) with SB scores of 0.35-0.55. Consistent with the in vivo data, all the tested mutant ribosomes showed decreased stalling at the tnaC stop codon in comparison with the wt ribosome during cell-free translation (FIG. 4g and FIG. 16), revealing their ability to more efficiently terminate translation of the TnaC peptide. The location of the identified termination arrest-releasing mutations suggests that either an altered placement of the peptidyl-tRNA or a less strict positioning of the P-site substrate and/or the release factor in the PTC of the mutant ribosome facilitate TnaC release. Arguably, the mutations that relieve TnaC-mediated termination arrest could be possibly isolated using the previous oRibo-T based approach^{13,15}. However, some of the mutations identified in OSYRIS would likely be missed, because the reduced expression level of the reporter afforded by oRibo-T in comparison with dissociable o-ribosomes in OSYRIS (FIG. 2d) would limit the number of mutants exceeding the minimal efficiency threshold imposed in our screen.

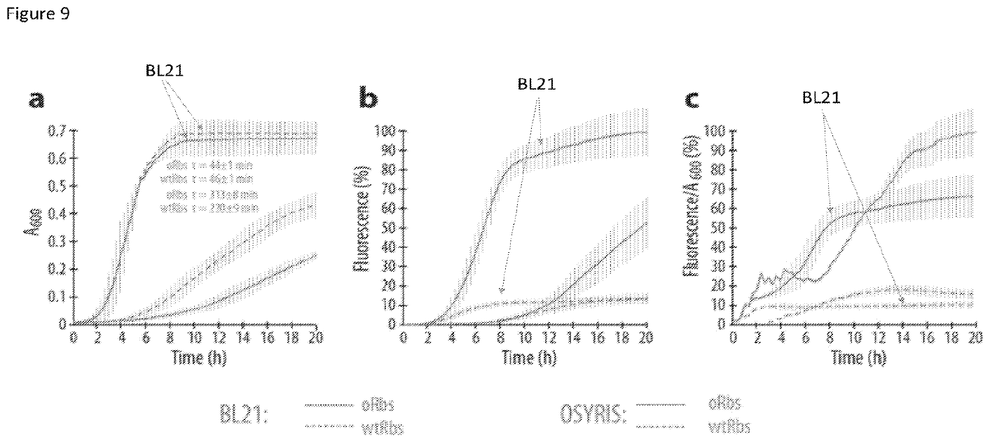

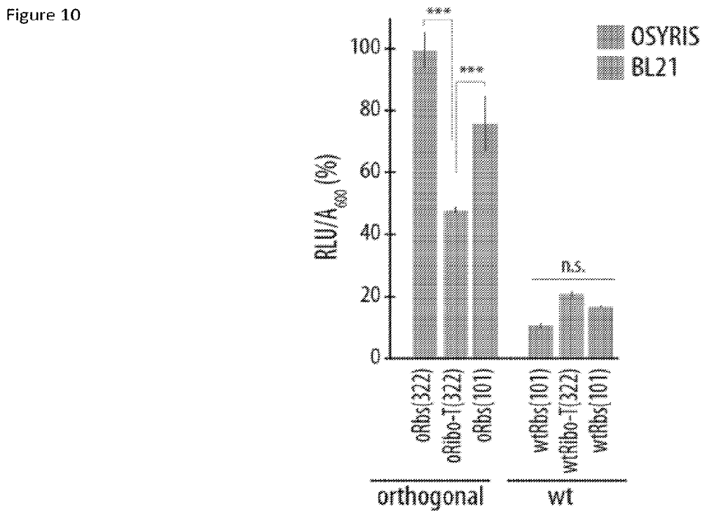

Our proof-of-principle experiments demonstrated that the OSYRIS design, based on the ability of Ribo-T to sustain cellular growth while compelling the dissociable subunits of the o-ribosome to interact with each other, presents a viable conceptually new approach for generating a fully-orthogonal cellular translation system. Engineering of OSYRIS was possible because Ribo-T is sufficiently active to translate the entire cellular proteome^{13,17}. However, translation driven by Ribo-T is sluggish and RiboT assembly is inefficient, which likely is one of the factors that contributes to the slow growth rate of the OSYRIS cells (doubling time τ˜300 min in 96-well plates in comparison with τ˜45 min for the BL21 strain) (FIG. 9a). Therefore, optimization of the Ribo-T functionality and assembly could improve the growth rate of OSYRIS cells and expand further the versatility of the orthogonal system. The three-plasmids set up (FIG. 5) makes OSYRIS highly modular and, thus, easily adjustable for various applications. In principle, OSYRIS could be simplified further by introducing Ribo-T rRNA genes into the chromosome and combining the orthogonal rRNA genes and the reporter gene on the same plasmid. Reducing the number of plasmids could additionally facilitate growth of the OSYRIS cells. Increasing the fraction of o-ribosomes in the OSYRIS cells by modulating either the plasmid copy number or the promoter strength could be another way to improve the system performance and adjust it to specific needs. Thus, while in our experiments o-ribosomes were engaged in expression of only a single reporter gene, several genes equipped with altered SD could be translated simultaneously if the fraction of o-ribosomes is properly balanced, opening the possibility of orthogonal expression of, for example, multi-subunit protein complexes.

An obvious possible application of OSYRIS is engineering ribosomes capable of incorporation of non-canonical amino acids into polypeptides that the ribosome discriminates against (such as backbond modified D- and Beta-amino acids³²). Although expanding ribosome's synthetic potential requires many components, from a specialized aminoacylation system to a designer genetic code, the fully orthogonal dissociable ribosome operating in the OSYRIS cells could accelerate the achievement of this goal. Importantly, OSYRIS makes possible many other endeavors, from employing ribosome retro-engineering for elucidating the origin of the translation apparatus to evolving new catalytic functions for programmable synthesis of polymers of non-protein nature.

### REFERENCES

1 Shine, J. & Dalgarno, L. The 3′-terminal sequence of Escherichia coli 16S ribosomal RNA: complementarity to nonsense triplets and ribosome binding sites. Proc. Natl. Acad. Sci. USA 71, 1342-1346 (1974).
2 Jenni, S. & Ban, N. The chemistry of protein synthesis and voyage through the ribosomal tunnel. Curr. Opin. Struct. Biol. 13, 212-219 (2003).
3 Rodnina, M. V. Translation in Prokaryotes. Cold Spring Harbor Perspect. Biol. 10, pii: a032664 (2018).
4 Des Soye, B. J., Patel, J. R., Isaacs, F. J. & Jewett, M. C. Repurposing the translation apparatus for synthetic biology. Curr. Opin. Chem. Biol. 28, 83-90 (2015).
5 Davis, L. & Chin, J. W. Designer proteins: applications of genetic code expansion in cell biology. Nat. Rev. Mol. Cell Biol. 13, 168-182 (2012).
6 Dedkova, L. M., Fahmi, N. E., Golovine, S. Y. & Hecht, S. M. Construction of Modified Ribosomes for Incorporation of d-Amino Acids into Proteins. Biochemistry 45, 15541-15551 (2006).
7 Ward, F. R., Watson, Z. L., Ad, O., Schepartz, A. & Cate, J. H. D. Defects in the assembly of ribosomes selected for beta-amino acid incorporation. Biochemistry, 58, 4494-4504 (2019).
8 Silverman, A. D., Karim, A. S. & Jewett, M. C. Cell-free gene expression: an expanded repertoire of applications. Nat. Rev. Genet., doi:10.1038/s41576-019-0186-3 (2019).
9 Arranz-Gibert, P., Vanderschuren, K. & Isaacs, F. J. The role of orthogonality in genetic code expansion. Life 9, 58 (2019).
10 Hui, A. & de Boer, H. A. Specialized ribosome system: preferential translation of a single mRNA species by a subpopulation of mutated ribosomes in Escherichia coli. Proc. Natl. Acad. Sci. USA 84, 4762-4766 (1987).
11 Rackham, O. & Chin, J. W. A network of orthogonal ribosome x mRNA pairs. Nat. Chem. Biol. 1, 159-166 (2005).
12 Wang, K., Neumann, H., Peak-Chew, S. Y. & Chin, J. W. Evolved orthogonal ribosomes enhance the efficiency of synthetic genetic code expansion. Nat. Biotechnol. 25, 770-777 (2007).
13 Orelle, C. et al. Protein synthesis by ribosomes with tethered subunits. Nature 524, 119-124 (2015).
14 Fried, S. D., Schmied, W. H., Uttamapinant, C. & Chin, J. W. Ribosome subunit stapling for orthogonal translation in E. coli. Angew. Chem. Int. Edit 54, 12791-12794 (2015).
15 Schmied, W. H. et al. Controlling orthogonal ribosome subunit interactions enables evolution of new function. Nature 564, 444-448 (2018).
16 Carlson, E. D. et al. Engineered ribosomes with tethered subunits for expanding biological function. Nat. Commun. 10, 3920 (2019).
17 Aleksashin, N. A. et al. Assembly and functionality of the ribosome with tethered subunits. Nat. Commun. 10, 930 (2019).
18 Quan, S., Skovgaard, O., McLaughlin, R. E., Buurman, E. T. & Squires, C. L. Markerless Escherichia coli rrn deletion strains for genetic determination of ribosomal binding sites. G3 5, 2555-2557 (2015).
19 Davis, J. H. et al. Modular assembly of the bacterial large ribosomal subunit. Cell 167, 1610-1622 (2016).
20 Sato, N. S., Hirabayashi, N., Agmon, I., Yonath, A. & Suzuki, T. Comprehensive genetic selection revealed essential bases in the peptidyl-transferase center. Proc. Natl. Acad. Sci. USA 103, 15386-15391 (2006).
21 Cochella, L. & Green, R. Isolation of antibiotic resistance mutations in the rRNA by using an in vitro selection system. Proc. Natl. Acad. Sci. USA 101, 3786-3791 (2004).
22 Nissen, P., Hansen, J., Ban, N., Moore, P. B. & Steitz, T. A. The structural basis of ribosome activity in peptide bond synthesis. Science 289, 920-930 (2000).
23 Polikanov, Y. S., Steitz, T. A. & Innis, C. A. A proton wire to couple aminoacyl-tRNA accommodation and peptide-bond formation on the ribosome. Nat. Struct. Molec. Biol. 21, 787-793 (2014).
24 Korostelev, A. A. Structural aspects of translation termination on the ribosome. RNA 17, 1409-1421 (2011).
25 Tate, W. P., Cridge, A. G. & Brown, C. M. ‘Stop’ in protein synthesis is modulated with exquisite subtlety by an extended RNA translation signal. Biochem. Soc. Trans. 46, 1615-1625 (2018).
26 Li, G. W., Burkhardt, D., Gross, C. & Weissman, J. S. Quantifying absolute protein synthesis rates reveals principles underlying allocation of cellular resources. Cell 157, 624-635 (2014).
27 Baggett, N. E., Zhang, Y. & Gross, C. A. Global analysis of translation termination in E. coli. PLoS Genet. 13, e1006676 (2017).
28 Gong, F. & Yanofsky, C. Instruction of translating ribosome by nascent peptide. Science 297, 1864-1867 (2002).
29 Martinez, A. K. et al. Interactions of the TnaC nascent peptide with rRNA in the exit tunnel enable the ribosome to respond to free tryptophan. Nucleic Acids Res. 42, 1245-1256 (2014).
30 Bischoff, L., Berninghausen, O. & Beckmann, R. Molecular basis for the ribosome functioning as an L-tryptophan sensor. Cell Rep. 9, 469-475 (2014).
31 Cruz-Vera, L. R., Rajagopal, S., Squires, C. & Yanofsky, C. Features of ribosome-peptidyl-tRNA interactions essential for tryptophan induction of tna operon expression. Mol. Cell 19, 333-343 (2005).
32 Lee, J. et al. Expanding the limits of the second genetic code with ribozymes. Nat. Commun. 10, 5097, doi:10.1038/s41467-019-12916-w (2019).

D. Materials and Methods

1. Assembly of the OSYRIS Cells

a. Plasmid Construction

Plasmids used for generation and optimization of the OSYRIS set-up are shown in FIG. 5. The nucleotide sequences and features of the key plasmids are shown in the Source data file.

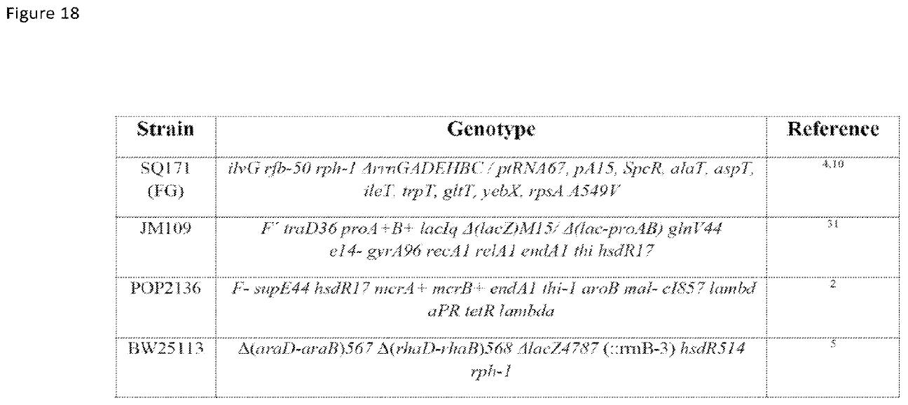

All the plasmids were constructed using Gibson assembly¹, with the plasmid backbone prepared by inverse PCR or restriction nuclease digest and the cloned inserts either PCR-amplified from the respective templates or synthesized chemically by Integrated DNA Technologies. PCR reactions were carried out using Q5 High-Fidelity DNA polymerase (New England Biolabs), and PCR products were purified using DNA Clean and Concentrator kit (Zymo Research). The Gibson assembly reactions for rRNA-encoding plasmids were electroporated into E. coli POP2136 cells (all the bacterial strains are listed in the Table at FIG. 18) and transformants were recovered on LB plates supplemented with the proper antibiotics; plates were incubated at 30° C. to prevent the expression of the rRNA genes controlled by the lambda P_{L} promoter². All the other plasmids were transformed and propagated in E. coli JM109 strain grown in LB media supplemented when needed with 100 μg/ml of ampicillin (Amp), 50 μg/ml of kanamycin (Kan) or 50 μg/ml of spectinomycin (Spc). Plasmids were isolated using High Pure Plasmid Isolation Kit (Roche) checked by PCR and capillary sequencing and used for engineering of the OSYRIS cells. The following sections outline the construction of the main plasmids.

i) pRibo-Tt Plasmid

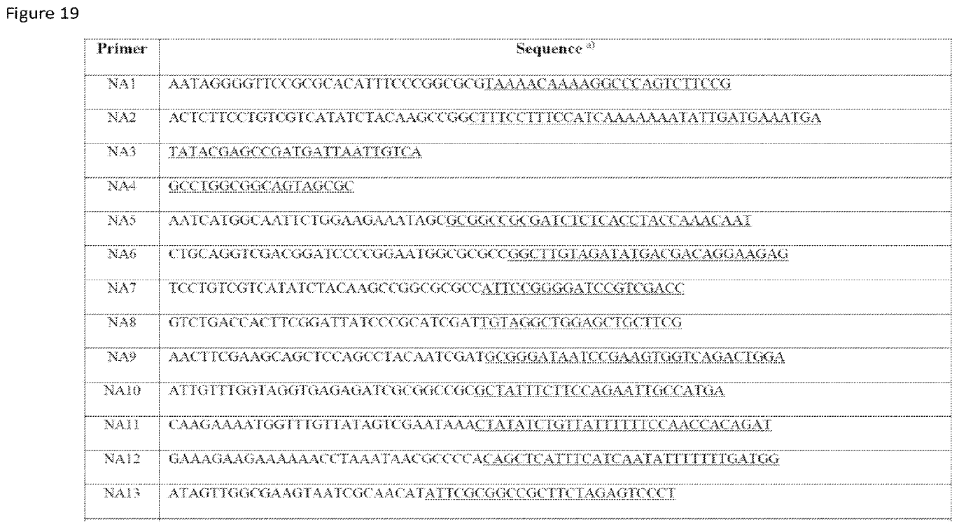

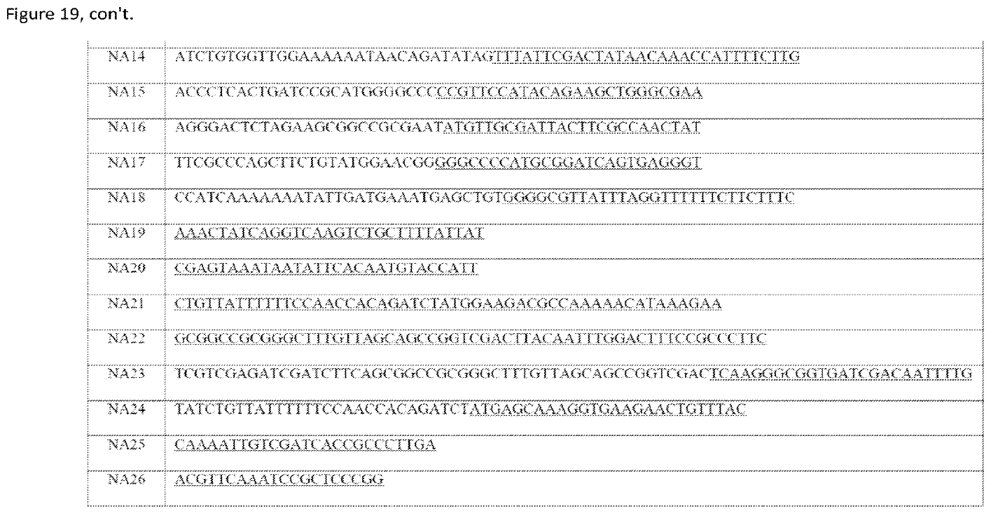

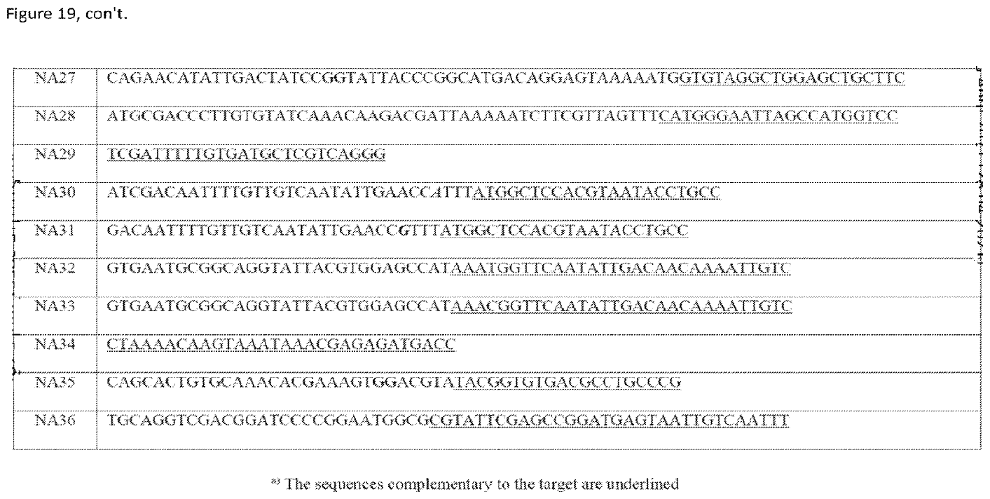

The backbone of the pRibo-T v 2.0 plasmid³, carrying the A2058G erythromycin resistance mutation, was linearized with SgsI restriction enzyme and purified. The cluster of the missing tRNAs genes (encoding tRNA^{Glu}, tRNA^{Ala}, tRNA^{Ile}, tRNA^{Trp} and tRNA^{Asp}), whose transcription is controlled by the P_{tac} promoter and T1 terminator, was synthesized as a gBlock (Integrated DNA Technology) and PCR-amplified using primers NA1 and NA2 (all primers are listed in the Table at FIG. 19). PCR reaction was catalyzed by the Q5 High-Fidelity DNA polymerase (New England Biolabs) according to the manufacture protocol under the following conditions: 98° C., 30 s followed by 30 cycles (98° C., 10 s; 64° C., 30 s; 72° C., 20 s), followed by the final incubation for 2 min at 72° C. Purified PCR product (20 ng) was mixed with SgsI-linearized pRibo-T v.2.0 backbone (80 ng) in a Gibson assembly reaction (1.7% PEG-800, 3.1 mM DTT, 0.31 mM β-nicotinamide adenine dinucleotide, 62.5 μM each dNTP, 3.1 mM MgCl₂, 31.3 mM Tris/HCl, pH 7.5, 0.004 U/μl T5 Exonuclease (Epicentre), 4 U/μl Taq DNA Ligase (New England Biolabs), 0.025 U/μl Phusion High Fidelity DNA Polymerase (New England Biolabs). After 1 h incubation at 50° C., 3 μl of the reaction mixture were transformed into electrocompetent POP2136 E. coli cells. Cells were plated onto LB/Amp agar plates. Individual colonies of the transformants were picked, grown onto LB/Amp media and plasmids were isolated. The presence of the tRNA cluster was confirmed by PCR amplification using primers NA3 and NA4 and sequencing.

ii) poRBS Plasmid

Orthogonal and wt rRNA operons under transcriptional control of the lambda P_{L} promoter and T1/T2 terminators were PCR amplified from the pO2 and pAM552 plasmids⁴, respectively, using the primers NA5 and NA6. The Kan^{R} gene was amplified from the plasmid pKD13⁵ using the primers NA7 and NA8. pSC101 origin of replication was amplified from the pCSacB plasmid⁶ using the primers NA9 and NA10. The PCR reactions were treated with DpnI to reduce the background of the parental plasmids. The PCR products were purified, confirmed by electrophoresis, and mixed (40 ng of each) in the Gibson assembly reaction. After 1 h incubation at 50° C., 3 μl of the reaction mix were transformed into electrocompetent POP2136 E. coli cells. Cells were plated onto LB/Kan agar plates. After 24 h incubation at 37° C., individual colonies were picked, grown in LB/Kan media, and plasmids were isolated and verified by restriction digest and sequencing.

iii) poGFP Plasmid

The o-GFP gene with 5′ UTR, 3′UTR, and T1/T2 terminators was PCR amplified from the plpp5-oGFP plasmid⁴ using primers NA11 and NA12. The LuxR repressor and the P_{Lux} promoter⁷ were PCR amplified from the pJDO75 plasmid⁸ using primers NA13 and NA14. Spc^{R} marker (aadA) was PCR amplified from the ptRNA67 plasmid⁶ using primers NA15 and NA16. The p15A origin of replication was PCR amplified from the ptRNA67 plasmid using primers NA17 and NA18. The PCR reactions involving plasmid templates were treated with DpnI. Purified PCR products (40 ng of each) were mixed in the Gibson assembly reaction. After 1 h incubation at 50° C. 3 μl of the reaction mix were transformed into electrocompetent JM109 E. coli cells (Promega). Cells were plated onto LB/Spc agar plates. After 24 h incubation at 37° C., individual colonies were picked, grown in LB/Spc media and plasmids were isolated. The presence of the luxR gene insert was confirmed by PCR using primers NA19 and NA20. Restriction digest of the resulting plasmid indicated that its size exceeds the expected one by ˜1 kb. Subsequent restriction analysis and sequencing showed that the luxR gene has undergone duplication (FIG. 5c). This duplication is not expected to affect the o-gfp reporter expression.

iv) poRFP/oGFP Plasmid

The Spc^{R} marker (aadA) and the p15A origin of replication were PCR amplified from the ptRNA67 plasmid⁶. The PCR reactions were treated with DpnI. The o-GFP gene with Pₗₚₚ₅ promoter, 5′ UTR, 3′UTR, and T1/T2 terminators was PCR amplified from the plpp5-oGFP plasmid⁴. Purified PCR products (˜40 ng of each) were mixed in the Gibson assembly reaction. After 1 h incubation at 50° C., 3 μl of the reaction mix were transformed into electrocompetent JM109 E. coli cells (Promega). Cells were plated onto LB/Spc agar plates. After 24 h incubation at 37° C., individual colonies were picked, grown in LB/Spc media and plasmids were isolated. The structure of the resulting plasmid plpp5-oGFP-pA15-Spec was verified by restriction digest and sequencing. The rfp gene with PT5 promoter and TO transcription terminator was PCR-amplified from the plasmid pRYG⁹, the orthogonal SD sequence was introduced by PCR and the resulting o-rfp construct was inserted into a unique SphI site of the plpp5-oGFP-pA15-Spc plasmid.

v) poLuc Plasmid

The plasmid poLuc carrying the orthogonal luciferase gene was constructed based on poGFP (FIG. 5c). The 1653 bp gene luc encoding firefly luciferase was PCR amplified from the pBESTluc plasmid (Promega) using the primers NA21 and NA22. The resulting PCR product and the poGFP plasmid were cut with restriction enzymes BglII and SalI and ligated. The ligation mixture was transformed into E. coli JM109 competent cells, the luc gene-positive clones were identified by colony PCR, and the integrity of the cloned luc gene was verified by sequencing.

vi) poGFP-TnaC Plasmid

For constructing the reporter poGFP-TnaC plasmids (wt or W12R mutant), the gfp-coding sequence in the poGFP plasmid was replaced with the sequences coding for the chimeric wt or mutant GFP-TnaC proteins. The DNA inserts containing the orthogonal ribosome binding site and GFP-TnaC or GFP-TnaC (W12R) coding sequences were generated by PCR using the templates used for in vitro translation (described below) using primers NA23 and NA24. After purification, the inserts were introduced by Gibson assembly into the poGFP plasmid cut with the restriction enzymes BglII and Salt After transformation, the presence of the correct insert in individual colonies was checked by colony PCR using the primers NA25 and NA26 and by sequencing the corresponding segments of the plasmid.

b. Engineering Ribo-T-Expressing Cells

SQ171 FG cells (Table at FIG. 18) that lack chromosomal rRNA alleles¹⁰ and carry mutations in the ybeX and rpsA genes that stimulate their growth when expressing Ribo-T⁴ were used as the host (FIG. 6). The gene upp was inactivated by recombineering for the future possible use of 5-fluorouracil negative selection.

The recipient cells initially carried two plasmids: the pCSacB plasmid containing the rrnB operon, counter-selectable sacB marker, and Kan^{R} gene, and the ptRNA67 plasmid carrying the missing tRNA genes that were eliminated during deletion of the chromosomal rRNA operons⁶. Cells were made electrocompetent and then 50 μl of the cell suspension were transformed with 50 ng of the pRibo-Tt plasmid, carrying the Ribo-T rRNA genes and missing tRNA genes (FIG. 5), isolated from the POP2136 cells. Transformed cells were diluted with 1 ml of SOC medium (2% tryptone, 0.5% yeast extract, 10 mM NaCl, 10 mM MgSO₄, 10 mM MgCl₂, 20 mM glucose) and incubated at 37° C. for 6 h with shaking. A 150 μl aliquot of the culture was diluted to 2 ml with fresh SOC medium supplemented with 50 μg/ml Amp, 25 μg/ml Spc, and 0.25% sucrose, and grown for 12 h at 37° C. with constant shaking. Cells were spun down (1 min, 5000 g) and plated on LB/agar plates containing 50 μg/ml Amp, 25 μg/ml Spc, 5% sucrose and 1 mg/ml erythromycin (Ery). Plates were incubated for 48 h at 37° C. The absence of the pCSacB plasmid was verified by the sensitivity of the transformants to Kan that was tested by replica plating colonies on LB/agar plates supplemented with 50 μg/ml Amp, 25 μg/ml Spc with or without the addition of 50 μg/ml of Kan. Transformants were then grown in LB media supplemented with 50 μg/ml Amp and 25 μg/ml Spc, plasmids were isolated and verified by restriction analysis. The absence of the wt rRNA was additionally confirmed by isolation of the total RNA using the RNeasy Mini Kit (Qiagen) and agarose gel electrophoresis.

c. Elimination of the ptRNA67 Plasmid

The obtained transformants were then cured of the ptRNA67 plasmid. For that, the cells were passaged in LB media supplemented with 100 μg/ml Amp for ˜100 generations. After plating cell dilutions, the absence of the ptRNA67 plasmid in individual clones was verified by their sensitivity to Spc and the lack of visible amounts of the ptRNA67 plasmid bands in the restriction digest of the total plasmid preparation.

d. Inactivation of the recA Gene in the Ribo-T-Expressing Cells

Our initial attempts to introduce poRbs into engineered cells frequently led to the appearance of the aberrant plasmids resulting from recombination between the poRbs and pRibo-Tt plasmids. Therefore, to avoid this problem, we inactivated the recA gene in the cells bearing the pRibo-Tt plasmid. (Of note, inactivating the recA gene before curing off the ptRNA67 plasmid prevented the plasmid loss even after prolonged passaging of the cells in the absence of Spc).

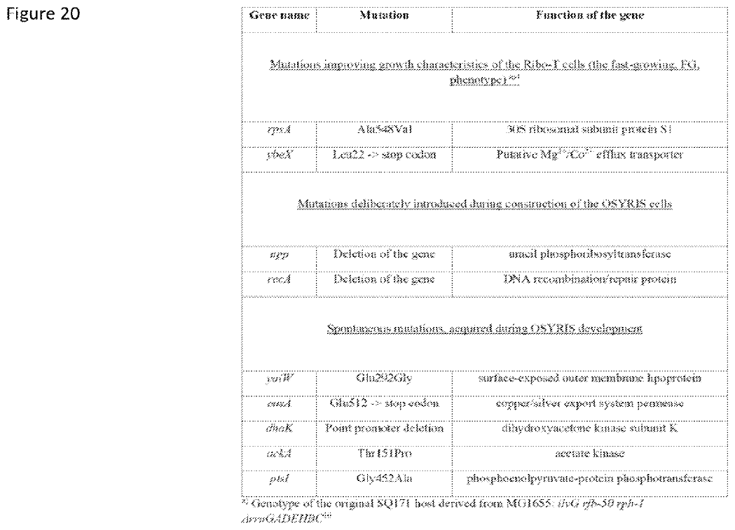

To inactivate the recA gene in the OSYRIS cells by P1 phage transduction, we first prepared the donor strain BW25113 recA::cat by the conventional recombineering procedure using chloramphenicol (Chl)-resistance cassette from the pKD3 plasmid⁵. The cassette was PCR-amplified using the primers NA27 and NA28. PCR fragment was transformed into BW25113 strain carrying the Red recombinase-expressing plasmid pDK46. After the selection and verification of the recA::cat strain, and curing the pKD46 plasmid, the resulting strain was used as a donor for the phage transduction. P1 phages transduction was carried out according to the standard protocol¹¹ except that the recovery incubation was 6 h instead of 1 h before plating the transductants on LB/agar plates supplemented with 50 μg/ml Amp and 15 μg/ml Chl. The genotype of the engineered strain is shown in the table at FIG. 20.

e. Introduction of the poRbs Plasmid

The SQ171 FG ΔrecA/pRibo-Tt strain was then transformed with the poRbs (or when needed, pRbs) plasmid by electroporation and selection of the Amp^{R}/Kan^{R}/Chl^{R} cells. The only deviation from the standard transformation protocol was that recovery of the transformants in the SOC medium lacking antibiotics was prolonged to 6 h prior and transformants were selected on LB/agar plates supplemented with 50 μg/ml Amp, 25 μg/ml Kan and 15 μg/ml Chl. Transformants were verified by restriction analysis of the total plasmid and analysis of rRNA by agarose gel electrophoresis.

f. Introduction of the Reporter Plasmids

Reporter plasmids (poGFP, poRFP/oGFP, poLuc, poGFP-TnaC) were introduced by electroporation into SQ171 FG ΔrecA/pRibo-Tt/poRbs cells and selection of the Amp^{r}/Kan^{r}/Chl^{r}/Spe^{r} cells, essentially as described in the previous section.

g. Verifying the Genome Sequence of the OSYRIS Cells

During the construction of the OSYRIS cells, the original host cells have been passaged multiple times and undergone single-colony purification at multiple steps, possibly leading to the accumulation of spontaneous mutations. Therefore, the total genome of the fully assembled OSYRIS cells was sequenced. Analysis of the resulting sequence showed the presence of mutations in several genes (Table at FIG. 20). Some of these mutations (e.g., in the genes ptsI or ackA) may potentially negatively affect cell growth under some conditions and could be corrected in the future by genome engineering.

h. Monitoring the In Vivo Expression of the Orthogonal gfp Gene

The OSYRIS cells carrying either poRbs or pRbs plasmids (expressing orthogonal or non-orthogonal ribosomes, respectively) and the poGFP reporter plasmid were grown overnight in LB media supplemented with 50 μg/ml Amp, 25 μg/ml Kan, 25 μg/ml Spc and 15 μg/ml Chl at 37° C. with constant shaking. Cultures were diluted 1:40 (v/v) in fresh LB media supplemented with the same antibiotics and additionally containing 1 ng/ml of N-(β-ketocaproyl)-L-homoserine lactone (HSL) (Santa Cruz Biotechnology), the inducer of the reporter gene transcription. The cultures (120 μl) were placed in the wells of the 96-well flat-bottom polystyrene tissue culture plate (Costar) and placed in the plate reader (TECAN Infinite M200 Pro) and incubated at 37° C. with constant linear (3 mm) shaking. Cell culture densities (A600) and GFP fluorescence (an excitation wavelength of 485 nm, an emission wavelength of 520 nm, optimal gain 30% RFU with applying the gain regulation function) were monitored over a time period of 24-48 h. The autofluorescence of cells lacking the reporter was subtracted from all the recorded values.

For the erythromycin sensitivity test, overnight cultures were diluted 1:40 into fresh LB media supplemented with either only HSL (final concentrations: 0-16 ng/ml) or with 1 ng/ml of HSL and varying concentrations of erythromycin (final concentrations: 0-1 mg/ml). Monitoring of cell growth and GFP expression was as described in the previous paragraph.

When OSYRIS cells carried the poRFP/poGFP reporter, the expression of RFP was monitored using an excitation wavelength of 550 nm and an emission wavelength of 675 nm, optimal gain 30% RFU with applying the gain regulation function.

2. In Vivo Expression of the Orthogonal Luciferase Gene

The OSYRIS cells carrying the poLuc plasmid were grown for 24 h in LB media supplemented with 50 μg/ml Amp, 25 μg/ml Kan, 25 μg/ml Spc and 15 μg/ml Chl and then diluted 1:40 into fresh medium containing the same antibiotics and 1 ng/ml of HSL. After 6 hrs, 0.2 A₆₀₀ of each culture was spun down (5 min, 5000 g, 4° C.), and cell pellets were flash-frozen. Luciferase activity was measured using the Luciferase Assay System (Promega) following the manufacturer's protocol. Specifically: cell pellets were thawed in a 20° C. water bath and resuspended in a 25 μl of LB supplemented with 10% (v/v) of dibasic phosphate buffer (1 M K₂HPO₄ pH 7.8, 20 mM EDTA). 20 μl of cell suspension were mixed with 60 μl of freshly prepared lysis mix (25 mM Tris-phosphate pH 7.8, 2 mM dithiothreitol, 2 mM 1,2-diaminocyclohexane-N,N,N′,N′-tetraacetic acid, 10% glycerol, 1% Triton X-100, 1.25 mg/ml lysozyme, 2.5 mg/ml bovine serum albumin) and lysed at room temperature for 10 min. The 10 μl aliquots of cell lysates were then placed into wells of 96-well black/clear bottom assay plate (Corning), 50 μl of Luciferase Assay Reagent (Promega) were added and fluorescence readings were immediately acquired in TECAN microplate reader.

3. Comparison of the Reporter Expression Driven by oRibo-T or oRbs

E. coli BL21 strain was transformed with either poGFP or poLuc plasmids. The transformants were selected on LB/agar plates supplemented with 50 μg/ml of Spc, grown from individual colonies, and then rendered electrocompetent. The reporter-containing cells were then transformed with poRibo-T (the pBR322 ori-based, Amp^{R} plasmid expressing oRibo-T rRNA)³, or with o-pAM552 plasmid (the pBR322 ori-based, Amp^{R} plasmid expressing oRbs rRNA)³.

The expression of o-gfp or o-luc reporters was measured as described above.

4. Analysis of the Mutant rRNA Content

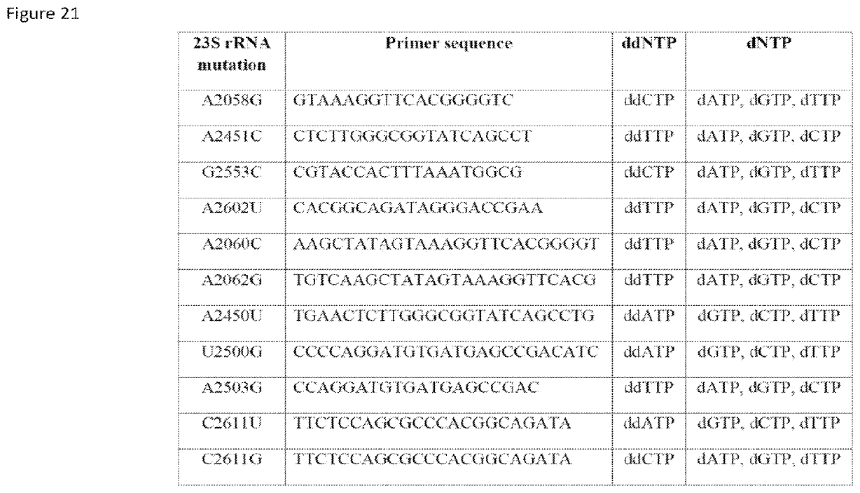

The presence of the engineered mutations in the 23S rRNA of the orthogonal ribosome was analyzed by primer extension. For that, total RNA was isolated from the OSYRIS cells using the RNeasy Mini Kit (Qiagen). The primers and combination of dNTPs and ddNTPs for analysis of each mutation are shown in the table at FIG. 21. For each assay, the appropriate 5′ [³²P]-labeled primer (0.5 pmol) was annealed to 1 μg of total RNA in 1× hybridization buffer (50 mM K-HEPES, pH 7.0, 100 mM KCl) by incubating at 90° C. for 1 min and then cooling over 15 min to 42° C. Annealed primers were extended with 2 units of AMV reverse transcriptase (Roche) in the presence of 0.25 mM of the appropriate ddNTP and 0.2 mM of each of the remaining dNTPs (Table at FIG. 21) for 20 min at 42° C. (final reaction volume of 8 μl). The reaction was stopped by adding 120 μl of stop buffer (84 mM NaOAc, 0.8 mM EDTA, pH 8.0, 70% EtOH), cooling at −80° C. for 15 min and pelleting nucleic acids by centrifugation 1 h at 15000 g (4° C.). The supernatant was removed, the pellet was dried and dissolved in formamide loading dye. The cDNA products were resolved in a 12% denaturing polyacrylamide gel and visualized by phosphorimaging. The intensity of the toeprint bands was determined using the ImageJ software¹². The background was subtracted.

5. Expression of the GFP-TnaC (wt) or GFP-TnaC(W12R) Proteins in the Cell-Free Translation System

The DNA templates containing the T7 RNA polymerase promoter, ribosome binding site from bacteriophage T7 gene 10 and GFP-TnaC or GFP-TnaC (W12R) coding sequences (see Appendix I) were generated by cross-over PCR. First, the T7 promoter and the gfp-coding sequence were PCR amplified from the pY71-T7-GFP plasmid¹³ using the T7 promoter forward primer NA29 (Table at FIG. 19) and either NA30 complementary to the wt tnaC or NA31 complementary to the W12R mutant of the tnaC gene. Independently, 3′segments of the wt or mutant tnaC genes with the 3′ untranslated regions were PCR amplified from the plasmids pGF2500-tnaC-wt or pGF2500-tnaC-mut¹⁴ using forward primers NA32 for wt, or NA33 for the W12R mutant, and a common reverse primer NA34.

Two PCR products corresponding to either the wt or mutant gfp-tnaC constructs were then combined together at 400 pg/μl (final concentration) and reamplified using the T7 and TnaC(rev) primers.

In vitro translation of the gfp-tnaC templates was carried out in the PURExpress, ΔRibosome, ΔtRNAs, Δ amino acids cell-free translation system composed of purified components (New England Biolabs), as described in¹⁵ with minor modifications. Reactions were supplemented with a 19-amino acids mixture (final concentration: 0.3 mM of each amino acid) and L-tryptophan to a final concentration of 50 μM (for reactions with low-tryptophan conditions) or 5 mM Trp (for high-tryptophan conditions). PCR-generated DNA templates were added to a final concentration of 5 ng/μl. The reactions were carried out at 37° C. for 3 h in a total volume of 5 μl in 384-well plates with black walls and clear bottom (Falcon) in a plate reader (TECAN Infinite M200 Pro). GFP fluorescence (excitation at 485 nm, emission at 520 nm, optimal gain 30% RFU with applying the gain regulation function) was monitored over time.

6. Preparation of the PTC Mutant Library

The PTC mutant library was generated by transferring individual mutations from the pT7rrnB library¹⁶ into the 23S rRNA gene in the poRbs plasmid.

To prepare the plasmid backbone, the poRbs plasmid was digested with SgsI and Bst1107I restriction enzymes, resulting in the excision of a 1546 nt fragment from the 23S rRNA gene. The reaction products were separated by agarose gel electrophoresis, and the 7483 bp backbone fragment was purified from the gel using Zymoclean Gel DNA Recovery Kit (Zymo Research) and DNA Clean & Concentrator Kit (Zymo Research) sequentially.

To generate the 1606-bp inserts carrying the PTC mutations, individual plasmids of the pT7rrnB plasmid library were used as a template for the PCR reaction catalyzed by the Q5 High-Fidelity DNA Polymerase (New England Biolabs) and employing the primers NA35 and NA36. PCR products were cleaned up using the DNA Clean & Concentrator Kit (Zymo Research).

The plasmid backbone (35 ng) and the DNA inserts (60 ng) were mixed in a total volume of 5 μl of a Gibson assembly reaction and incubated for 1 h at 50° C.

Individual Gibson-assembly reactions were used to transform chemically-competent POP2136 cells. The high-throughput transformation was carried out in a flat-bottom tissue culture 96-well plates with low evaporation transparent lid (Falcon). In each well of the plate, 20 μl of competent cells were mixed with 2 μl of individual Gibson assembly reactions. Plates were incubated on ice for 30 minutes, at 42° C. for 50 s and again on ice for 15 minutes. One hundred μl of SOC medium were added to each well, and cells were allowed to recover at 30° C. for 2 h on a shaker. Culture volumes were reduced to 40 μl by spinning the plate at 6000 g for 6 min in a swinging bucket rotor and removing 80 μl of supernatant. Six μl of each of the remaining cell suspension were then spot-plated using a multi-channel pipettor on LB/agar rectangular OmniTray Single-Well plates (Nunc) supplemented with 50 μg/ml Kan. Plates were incubated at 30° C. for 20 h.

Individual colonies were inoculated in fresh LB media supplemented with 50 μg/ml Kan and grown for 12 h at 30° C. Plasmids were isolated, and the presence of the desired mutations, as well as the lack of off-target mutations in the PCR-amplified 23S rRNA segments, were confirmed by capillary sequencing.

The individual PTC mutant library plasmids were then introduced into OSYRIS cells by transforming them into SQ171 FG/pRibo-Tt/poGFP-TnaC cells using the high-throughput transformation approach described above with the following modifications: i) 20 ng of the purified individual plasmids were used in transformation; ii) transformants were recovered in SOC medium for 6 h at 37° C. and patched onto LB/agar plates supplemented with 50 μg/ml Amp, 25 μg/ml Kan, 25 μg/ml Spc, and 15 μg/ml Chl; iii) plates were incubated at 37° C. for 48 h; iv) glycerol stocks were prepared in 96-well plates from cultures grown from individual colonies of the transformants.

7. PTC Library Screening

Individual colonies of the OSYRIS cells carrying the PTC library mutants were inoculated in the wells of a 96-well plate containing 120 μl of LB media, supplemented with 50 μg/ml Amp, 25 μg/ml Kan, 25 μg/ml Spc, and 15 μg/ml Chl, and grown for 24 h at 37° C. with constant shaking. Cultures were diluted 1:40 (v/v) in 120 μl of fresh LB supplemented with the same antibiotics, 0.35 mg/ml 1-methyl-tryptophan (Sigma) and 0.016 ng/ml of HSL. Plates were placed into TECAN Infinite M200 Pro plate reader and incubated at 37° C. with constant linear (3 mm) shaking. Optical density (A₆₀₀) of the cultures and oGFP fluorescence were monitored as described above.

The termination arrest bypass score was calculated by comparing the efficiency of GFP expression in the OSYRIS cells carrying GFP-TnaC(W12R) mutant construct to that in the OSYRIS cells carrying wt GFP-TnaC construct. The stalling bypass (SB) score values were computed based on the readings obtained at the 48 h time point using the following formula:

$$Bypass score = \frac{RFU ( WT ) / {A}_{600} ( WT )}{RFU ( W 12 R ) / {A}_{600} ( W 12 R )}$$

where RFU is relative fluorescence units.

The mean SB score values were calculated using data obtained in two independent experiments.

8. Isolation of the 50S Ribosomal Subunits from the OSYRIS Cells

The ribosomes were isolated from the OSYRIS cells following the protocol described by Ohashi et al¹⁷. Specifically, OSYRIS cells expressing ribosomes with mutations U2500G, A2060C or A2450U in 23S rRNA were grown overnight at 37° C. in LB medium supplemented with 50 μg/ml Amp, 25 μg/ml Kan, 25 μg/ml Spc, and 15 μg/ml Chl. The cultures were diluted to the final A₆₀₀=0.003 into 1 L of fresh LB media supplemented with the same antibiotics and grown for approximately 15 h with vigorous shaking until optical density reached A₆₀₀=0.35. Cells were collected by centrifugation for 15 min at 5000 g (4° C.), and cell pellets were flash-frozen in liquid nitrogen and stored at −80° C. Frozen cell pellets were resuspended in 20 ml of lysis buffer (10 mM HEPES-KOH, pH 7.6, 50 mM KCl, 10 mM Mg(OAc)₂, 7 mM β-mercaptoethanol), lysed in EmulsiFlex-C3 homogenizer (AVESTIN Inc.) at 15000 psi for 5 min and then lysates were clarified by 30 min centrifugation at 20000 g (4° C.) and transferred to new centrifuge tubes. Ammonium sulfate was added to the final concentration of 1.5 M and tubes were centrifuged for 1 h at 20000 g (4° C.). The ribosome (Ribo-T+dissociable ribosomes)-containing supernatant was filtered through a 0.22-μm Ø 30 mm polyethersulfone (PES) membrane filter (CELLTREAT Scientific Products). Ribosome material was purified by hydrophobic chromatography using a 5 ml HiTrap Butyl FF column (GE Healthcare Life Sciences), equilibrated with 20 mM HEPES-KOH, pH 7.6, 10 mM Mg(OAc)₂, 7 mM β-mercaptoethanol, 1.5 M (NH₄)₂SO₄, on an AKTApurifier UPC 10 (GE Healthcare). After loading the material, the column was washed with 20 mM HEPES-KOH, pH 7.6, 10 mM Mg(OAc)₂, 7 mM β-mercaptoethanol, 1.2 M (NH₄)₂SO₄, and the ribosomes were then eluted with the buffer containing 20 mM HEPES-KOH, pH 7.6, 10 mM Mg(OAc)₂, 7 mM β-mercaptoethanol, 0.75 M (NH₄)₂SO₄. Eluate fractions containing ribosomes were pulled together and loaded onto 16 ml 30% sucrose cushion prepared in the buffer 20 mM HEPES-KOH, pH 7.6, 10 mM Mg(OAc)₂, 30 mM NH₄Cl, 7 mM β-mercaptoethanol in 35 ml centrifuge tubes. Ribosomes were pelleted by centrifugation at 36000 rpm for 18 h at 4° C. in the Type 70 T1 rotor (Beckman). Ribosome pellets were resuspended in the dissociation/storage buffer (20 mM HEPES-KOH pH 7.6, 30 mM KCl, 1.5 mM Mg(OAc)₂, 7 mM β-mercaptoethanol) and aliquots were flash-frozen and stored at −80° C.

To isolate individual 50S ribosomal subunits, the ribosome preparations were loaded on 10-40% sucrose gradients prepared in buffer 20 mM Tris-HCl, pH 7.5, 1.5 mM Mg(OAc)₂, 100 mM NH₄Cl, 2 mM β-mercaptoethanol in the centrifuge tubes for the SW41 rotor (Beckman). The gradients were centrifuged for 16 h at 27000 rpm at 4° C. and fractionated on a gradient fractionator (BioComp) with A₂₅₄ monitoring. Fractions corresponding to the large ribosomal subunits were pooled, concentrated on Vivaspin 2 ml concentrators with cellulose triacetate membrane (Sartorius Stedim Biotech GmbH) and recovered in the ribosome storage buffer (20 mM HEPES-KOH pH 7.6, 30 mM KCl, 6 mM Mg(OAc)₂, 7 mM β-mercaptoethanol). The aliquots were flash-frozen and stored at −80° C.

9. Isolation of Ribosomes with Non-Lethal 23S rRNA Mutations

Ribosomes carrying non-lethal mutations in the 23S rRNA (A2503G, A2062G, C2611G, and C2611U) were isolated from the SQ171 cells carrying pAM552 plasmids⁴ with the corresponding mutations. The corresponding strains expressing pure populations of the mutant ribosomes were prepared as described previously¹⁸. The ribosomes were isolated as described above except that after sucrose cushion centrifugation, the ribosomal pellets were resuspended in the ribosome storage buffer (20 mM HEPES-KOH pH 7.6, 30 mM KCl, 6 mM Mg(OAc)₂, 7 mM β-mercaptoethanol). The aliquots were flash-frozen and stored at −80° C.

10. Toe-Printing Analysis

Primer extension inhibition (toeprinting) analysis¹⁹ was performed as described previously²⁰. When needed, the prolyl-tRNA synthetase inhibitor 5′-O-[N-(L-prolyl)-sulfamoyl] adenosine (L-PSA)²¹ was added to the reactions to the final concentrations of 50 μM. After separation of the primer extension products in the sequencing gel and phosphorimaging, the intensity of the toeprint bands was determined using the ImageJ software¹². The efficiency of the TnaC-induced translation arrest at the tnaC stop codon was calculated by comparing the intensity of the stop codon toeprint band (SB) (arrowhead in FIG. 16c) with the intensity of the toeprint band at the preceding codon in the L-PSA-containing samples (PB) (open arrowhead in FIG. 16c) using the formula:

$$TnaC - induced translation arrest = \frac{SB - {SB}_{BG}}{PB - {PB}_{BG}} * 100$$

where SB_{BG} and PB_{BG} are backgrounds for the corresponding bands.

11. Structural Analysis and Figure Preparation

For calculating the distances of the 23S rRNA nucleotides to the attacking a-amino group of the A-site amino acid, the crystal structure of the Thermus thermophilus ribosomes with P- and A-site tRNAs in the pre-attack state (PDB 1VY4)²² were aligned on the basis of the full-length 23S rRNA with the high-resolution structure of the partially rotated vacant E. coli ribosome (PDB 4YBB)²³. The distance measurements and figure rendering were performed in PyMOL (Molecular Graphics System, Version 2.0 Schrödinger, LLC.). FIG. 4g was prepared by aligning the cryo-EM structure of the E. coli ribosomes stalled with the TnaC-tRNA in the P site (PDB 4UY8)²⁴ with the crystallographic structure of T. thermophilus ribosome complexed with RF2 (PDB 4V67)²⁵.

12. Statistical Analysis

Where relevant, statistical values can be found in the figure legends. The mean of the value was defined as the arithmetic mean. Depending on the numbers of the independent biological replicates (n), deviation ranges represent either standard deviation (s.d.) (n≥3) or experimental error (n=2). All statistical values were calculated and all graphs were plotted using the Microsoft Excel 365 software. The Student's t-test was performed using GraphPad Prism version 8.00 for Windows (GraphPad Software, La Jolla Calif. USA).

### REFERENCES FOR MATERIALS AND METHODS

1 Gibson, D. G. et al. Enzymatic assembly of DNA molecules up to several hundred kilobases. Nat Methods 6, 343-345 (2009).
2 Kusters, J. G., Jager, E. J. & van der Zeijst, B. A. Improvement of the cloning linker of the bacterial expression vector pEX. Nucleic Acids Res 17, 8007 (1989).
3 Carlson, E. D. et al. Engineered ribosomes with tethered subunits for expanding biological function. Nat Commun 10, 3920 (2019).
4 Orelle, C. et al. Protein synthesis by ribosomes with tethered subunits. Nature 524, 119-124 (2015).
5 Datsenko, K. A. & Wanner, B. L. One-step inactivation of chromosomal genes in Escherichia coli K-12 using PCR products. Proc Natl Acad Sci USA 97, 6640-6645 (2000).
6 Zaporojets, D., French, S. & Squires, C. L. Products transcribed from rearranged rrn genes of Escherichia coli can assemble to form functional ribosomes. J Bacteriol 185, 6921-6927 (2003).
7 Canton, B., Labno, A. & Endy, D. Refinement and standardization of synthetic biological parts and devices. Nat Biotechnol 26, 787-793 (2008).
8 Davis, J. H. et al. Modular assembly of the bacterial large ribosomal subunit. Cell 167, 1610-1622 (2016).
9 Monk, J. W. et al. Rapid and inexpensive evaluation of nonstandard amino acid incorporation in Escherichia coli. ACS Synth Biol 6, 45-54 (2017).
10 Quan, S., Skovgaard, O., McLaughlin, R. E., Buurman, E. T. & Squires, C. L. Markerless Escherichia coli rrn deletion strains for genetic determination of ribosomal binding sites. G3 5, 2555-2557 (2015).
11 Thomason, L. C., Costantino, N. & Court, D. L. E. coli genome manipulation by P1 transduction. Curr Protoc Mol Biol/edited by Frederick M Ausubel . . . [et al.] Chapter 1, Unit 1 17 (2007).
12 Schneider, C. A., Rasband, W. S. & Eliceiri, K. W. NIH Image to ImageJ: 25 years of image analysis. Nat Methods 9, 671-675 (2012).
13 Bundy, B. C. & Swartz, J. R. Site-specific incorporation of p-propargyloxyphenylalanine in a cell-free environment for direct protein-protein click conjugation. Bioconjug Chem 21, 255-263 (2010).
14 Gong, F. & Yanofsky, C. Reproducing tna operon regulation in vitro in an S-30 system. Tryptophan induction inhibits cleavage of TnaC peptidyl-tRNA. J Biol Chem 276, 1974-1983 (2001).
15 Martinez, A. K. et al. Interactions of the TnaC nascent peptide with rRNA in the exit tunnel enable the ribosome to respond to free tryptophan. Nucleic Acids Res 42, 1245-1256, doi:10.1093/nar/gkt923 (2014).
16 d'Aquino, A. E., Azim, T., Aleksashin, N. A., Hockenberry, A. H., Jewett, M. C. Mutating the ribosomal peptidyl transferase center in vitro. Nucleic Acids Res., in press (2020).
17 Ohashi, H., Shimizu, Y., Ying, B. W. & Ueda, T. Efficient protein selection based on ribosome display system with purified components. Biochem Biophys Res Commun 352, 270-276 (2007).
18 Vazquez-Laslop, N., Thum, C. & Mankin, A. S. Molecular mechanism of drug-dependent ribosome stalling. Mol Cell 30, 190-202 (2008).
19 Hartz, D., McPheeters, D. S., Traut, R. & Gold, L. Extension inhibition analysis of translation initiation complexes. Methods Enzymol 164, 419-425 (1988).
20 Orelle, C. et al. Identifying the targets of aminoacyl-tRNA synthetase inhibitors by primer extension inhibition. Nucleic Acids Res 41, e144 (2013).
21 Heacock, D., Forsyth, C. J., Shiba, K. & MusierForsyth, K. Synthesis and aminoacyl-tRNA synthetase inhibitory activity of prolyl adenylate analogs. Bioorg Chem 24, 273-289 (1996).
22 Polikanov, Y. S., Steitz, T. A. & Innis, C. A. A proton wire to couple aminoacyl-tRNA accommodation and peptide-bond formation on the ribosome. Nat Struct Mol Biol 21, 787-793 (2014).
23 Noeske, J. et al. High-resolution structure of the Escherichia coli ribosome. Nat Struct Mol Biol 22, 336-341 (2015).
24 Bischoff, L., Berninghausen, 0. & Beckmann, R. Molecular basis for the ribosome functioning as an L-tryptophan sensor. Cell Rep 9, 469-475 (2014).
25 Korostelev, A. et al. Crystal structure of a translation termination complex formed with release factor RF2. Proc Natl Acad Sci USA 105, 19684-19689 (2008).
26 Keseler, I. M. et al. The EcoCyc database: reflecting new knowledge about Escherichia coli K-12. Nucleic Acids Res 45, D543-D550 (2017).
27 Sato, N. S., Hirabayashi, N., Agmon, I., Yonath, A. & Suzuki, T. Comprehensive genetic selection revealed essential bases in the peptidyl-transferase center. Proc Natl Acad Sci USA 103, 15386-15391 (2006).
28 Gong, F. & Yanofsky, C. Instruction of translating ribosome by nascent peptide. Science 297, 1864-1867 (2002).
29 Cruz-Vera, L. R., Rajagopal, S., Squires, C. & Yanofsky, C. Features of ribosome-peptidyl-tRNA interactions essential for tryptophan induction of tna operon expression. Mol Cell 19, 333-343 (2005).
30 Vazquez-Laslop, N., Ramu, H., Klepacki, D., Kannan, K., Mankin, A. S. The key role of a conserved and modified rRNA residue in the ribosomal response to the nascent peptide. EMBO J. 29, 3108-3117 (2010)
31 Yanisch-Perron, C., Vieira, J. & Messing, J. Improved M13 phage cloning vectors and host strains: nucleotide sequences of the M13mp18 and pUC19 vectors. Gene 33, 103-119 (1985).
33 Gibson, D. G. et al. Enzymatic assembly of DNA molecules up to several hundred kilobases. Nat. Methods 6, 343-345 (2009).
34 Kusters, J. G., Jager, E. J. & van der Zeijst, B. A. Improvement of the cloning linker of the bacterial expression vector pEX. Nucleic Acids Res. 17, 8007 (1989).
35 Datsenko, K. A. & Wanner, B. L. One-step inactivation of chromosomal genes in Escherichia coli K-12 using PCR products. Proc. Nat.l Acad. Sci. USA 97, 6640-6645 (2000).
36 Zaporojets, D., French, S. & Squires, C. L. Products transcribed from rearranged rrn genes of Escherichia coli can assemble to form functional ribosomes. J. Bacteriol. 185, 6921-6927 (2003).
37 Canton, B., Labno, A. & Endy, D. Refinement and standardization of synthetic biological parts and devices. Nat. Biotechnol. 26, 787-793 (2008).
38. Davis, J. H. et al. Modular assembly of the bacterial large ribosomal subunit. Cell 167, 1610-1622 (2016)
39 Monk, J. W. et al. Rapid and inexpensive evaluation of nonstandard amino acid incorporation in Escherichia coli. ACS Synth. Bio.l 6, 45-54 (2017).
40 Thomason, L. C., Costantino, N. & Court, D. L. E. coli genome manipulation by P1 transduction. Curr. Protoc. Mol. Biol./edited by Frederick M. Ausubel . . . [et al.] Chapter 1, Unit 1 17 (2007).
41 Schneider, C. A., Rasband, W. S. & Eliceiri, K. W. NIH Image to ImageJ: 25 years of image analysis. Nat. Methods 9, 671-675 (2012).
42 Bundy, B. C. & Swartz, J. R. Site-specific incorporation of p-propargyloxyphenylalanine in a cell-free environment for direct protein-protein click conjugation. Bioconjug. Chem. 21, 255-263 (2010).
43 Gong, F. & Yanofsky, C. Reproducing tna operon regulation in vitro in an S-30 system. Tryptophan induction inhibits cleavage of TnaC peptidyl-tRNA. J. Biol. Chem. 276, 1974-1983 (2001).
44 d'Aquino, A. E., Azim, T., Aleksashin, N. A., Hockenberry, A. H., Jewett, M. C. Mutational characterization and mapping of the 70S ribosome active site. Nucleic Acids Res 48, 2777-2789 (2020).
45 Ohashi, H., Shimizu, Y., Ying, B. W. & Ueda, T. Efficient protein selection based on ribosome display system with purified components. Bioch. Biophys. Res. Commun. 352, 270-276 (2007).
46 Vazquez-Laslop, N., Thum, C. & Mankin, A. S. Molecular mechanism of drug-dependent ribosome stalling. Mol. Cell 30, 190-202 (2008).
47 Hartz, D., McPheeters, D. S., Traut, R. & Gold, L. Extension inhibition analysis of translation initiation complexes. Methods Enzymol. 164, 419-425 (1988).
48 Orelle, C. et al. Identifying the targets of aminoacyl-tRNA synthetase inhibitors by primer extension inhibition. Nucleic Acids Res. 41, e144 (2013).
49 Heacock, D., Forsyth, C. J., Shiba, K. & MusierForsyth, K. Synthesis and aminoacyl-tRNA synthetase inhibitory activity of prolyl adenylate analogs. Bioorg. Chem. 24, 273-289 (1996).
50 Noeske, J. et al. High-resolution structure of the Escherichia coli ribosome. Nat. Struct. Molec. Biol. 22, 336-341 (2015).
51 Korostelev, A. et al. Crystal structure of a translation termination complex formed with release factor RF2. Proc. Natl. Acad. Sci. USA 105, 19684-19689 (2008).

### ADDITIONAL REFERENCES

Rackham, O.; Chin, J. W., Compositions and methods relating to orthogonal ribosome mRNA pairs. U.S. Ser. No. 11/982,877: Filing date Nov. 6, 2007.
Chin, J.; Wang, K.; Neumann, H., Orthogonal Q-Ribosomes. U.S. Ser. No. 13/517,372: Filing date Dec. 20, 2010.
Chin, J.; Wang, K.; Neumann, H., Evolved orthogonal ribosomes. U.S. Ser. No. 12/516,230: Filing date Nov. 28, 2007.

E. Applications and Advantages of the “Flipped” Orthogonal Translation System of Example 1

1. Applications

By way of example, but not by way of limitation, applications of the compositions and methods disclosed herein include, but are not limited to: Ribosome evolution/engineering (for example towards more efficient non-canonical amino acid incorporation); Expanded genetic codes for non-canonical amino acid incorporation; Enabling detailed in vivo studies of antibiotic resistance mechanisms, enabling antibiotic development process; Biopharmaceutical production; Orthogonal circuits in cells; Synthetic biology; Producing engineered peptide by incorporating new functionality inaccessible to peptides synthesized by native (or wildtype) ribosome or their post-translationally modified derivatives; Producing novel protease-resistant peptides that could transform medicinal chemistry; Allows for the development of engineered ribosomes in cells.

2. Advantages

By way of example, but not by way of limitation, advantages of the compositions and methods disclosed herein include but are not limited to the following.

The unusual design of Ribo-T limits its functionality as an orthogonal translation system (oRiboT). Specifically, Ribo-T translates proteins with only half the rate of the dissociable ribosome. It is slower in departing from the start codons in comparison with the wt ribosomes. Furthermore, the biogenesis of even ‘wt’ Ribo-T is rather slow and inefficient and the assembly problems could be additionally exacerbated if the ribosome's functional centers are subjected to additional alterations.

In order to overcome the shortcomings of the original oRibo-T-based approach for engineering cells with two functionally-independent translation machineries, we have now created a conceptually new design of an in vivo system that utilizes dissociable, yet fully segregated, ribosomes dedicated to translation of only specialized mRNAs. By ‘flipping’ the roles of Ribo-T and dissociable ribosomes, we engineered bacterial cells where translation of the proteome is carried out by Ribo-T, whereas the ribosome, composed of the dissociable orthogonal 30S (o-30S) subunit and wt 50S subunit functions as a fully orthogonal translation machine. In the resulting setup, that we named OSYRIS (Orthogonal translation SYstem based on Ribosomes with Isolated Subunits), complete orthogonality is achieved because the tethered nature of Ribo-T precludes it from associating with either the o-30S or the 50S of the dissociable ribosome. Therefore, in OSYRIS cells, the physically-unlinked o-30S and 50S ribosomal subunits are nevertheless compelled to interact with each other and function as fully orthogonal ribosomes (o-ribosomes). As a result, not only the o-30S, but also the free 50S subunit can be engineered to achieve new functionalities without interfering with the expression of the cellular proteome.

When compared OSYRIS cells in a side by side the expression of two orthogonal reporters (o-gfp and the newly engineered o-luc) driven by either the dissociable orthogonal ribosomes (oRbs) or the orthogonal tethered ribosomes (oRibo-T) in the same host (E. coli BL21). Noteworthy, in spite of oRibo-T being expressed from a high copy number vector while oRbs was transcribed from a low copy number plasmid, oRbs outperformed oRibo-T. This result clearly demonstrates the advantage offered by oRbs over oRibo-T in translating orthogonal mRNAs and solidifies the notion that the OSYRIS design is superior to the one based on oRibo-T.

Ribosome engineering is of great interests to the fields of biotechnology, chemistry, and material science, but previous approaches have not been able to evolve the large subunit of the ribosome, which comprises the catalytic active site and the protein excretion tunnel. The development of a tethered ribosome removes these limitations and expands the possibilities of ribosome engineering. Ribosomes may be engineered to incorporate unnatural amino acids for expanded protein functionality or to perform new chemistry for the production of non-protein polymers.

This invention details the first ever orthogonal ribosome-mRNA system where mRNA decoding, catalysis of polypeptide synthesis, and protein excretion can all be optimized for new substrates and functions. The key difference from the prior art is that not only the small (decoding) ribosomal subunit, but also the large (catalytic) ribosomal subunit function as a single, combined and undividable orthogonal genetic synthetic machine.

Moreover, this is unique in that a tethered ribosome keeps the cell alive and a freely dissociable ribosome is used for engineering.

We stress that this invention represents the first of its kind. We anticipate that the innovations reported here will help to inspire larger ribosome construction and engineering efforts to push the limits of engineered biological systems, opening new commercial opportunities in research areas that are currently beyond the adjacent possible.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. An engineered cell comprising a first protein translation mechanism and a second protein translation mechanism,
a) the first protein translation mechanism comprising a first engineered ribosome, the first engineered ribosome comprising:
i) a small subunit comprising ribosomal RNA (rRNA) and protein;
ii) a large subunit comprising a ribosomal RNA (rRNA) and protein; and
iii) a linking moiety,
wherein the linking moiety comprises a polynucleotide sequence and tethers the rRNA of the small subunit with the rRNA of the large subunit;
b) the second protein translation mechanism comprising a second engineered ribosome, the second engineered ribosome comprising:
i) a small subunit comprising rRNA and protein; and
ii) a large subunit comprising rRNA and protein;
wherein the second engineered ribosome lacks a linking moiety between the large subunit and the small subunit; and
wherein the small subunit of the second engineered ribosome comprises a modified anti-Shine-Dalgarno sequence to permit translation of templates having complementary and/or cognate Shine-Dalgarno sequence different from endogenous cellular mRNAs of the cell, and/or

2. The engineered cell of claim 1, wherein the first and the second protein translation mechanisms are capable of supporting translation of a sequence defined polymer.

3. The engineered cell of claim 1, wherein the first protein translation mechanism is capable of supporting translation of native, endogenous RNAs.

4. The engineered cell of claim 1, wherein the second protein translation mechanism is capable of supporting translation of non-native, exogenous RNAs.

5. The engineered cell of claim 1, wherein the second engineered ribosome comprises one or more change-of-function mutations, wherein the change-of-function mutation is not at the anti-Shine Dalgarno sequence.

6. The engineered cell of claim 1, wherein the small subunit of the second engineered ribosome comprises a modified anti-Shine-Dalgarno sequence selected from the group consisting of 3′-GGUGUU-5′, 3′-UGGUGU-5′, 3′-GGUGUC-5′, 3′-GUUUAG-5′, 3′-UGGAAU-5′, 3′GGAUCU-5′, 3′-UGGAUC-5′, 3′-UGGUAA-5′, and 3′-UGGAUC-5′.

7. The engineered cell of claim 1, wherein the second engineered ribosome comprises a change-of-function mutation in one or more of:
a) peptidyl transferase center (PTC);
b) nascent peptide exit tunnel (NPET);
c) interaction site with elongation factors;
d) tRNA binding sites;
e) chaperone binding sites;
f) nascent chain modifying enzyme biding sites;
g) GTPase center.

8. The engineered cell of claim 1, wherein the large subunit of the second engineered ribosome comprises a change-of-function mutations at one or more of the following residues of a 23S rRNA: G2061, C2452, U2585, G2251, G2252, A2057, A2058, C2611, A2062, A2503, U2609, G2454, and G2455.

9. The engineered cell of claim 1, wherein the first, the second, or both the first and the second engineered ribosomes comprises an antibiotic resistance mutation.

10. The engineered cell of claim 1, wherein the large subunit of the first engineered ribosome comprises a permuted variant or mutant of a 23 SrRNA and/or the small subunit comprises a permuted variant or mutant of a 16S rRNA.

11. The engineered cell of claim 1, wherein the linking moiety covalently bonds a helix of the large subunit selected from the group consisting of helix 10, helix, 38, helix 42, helix, 54, helix 58, helix, 63, helix 78, helix, 101, to a helix of the small subunit selected from the group consisting of helix 11, helix, 26, helix 33, and helix 44.

12. A method for preparing a sequence-defined polymer, the method comprising:
(a) providing one or more of:
(i) the cell of claim 1;
(ii) a cell-free extract derived from the cells of claim 1;
(iii) purified translation system derived from the cell of claim 1;
b) providing an mRNA encoding the sequence-defined polymer to the cell or the cell-free extract; and
c) translating the mRNA in the cell or cell-free extract to provide the sequence-defined polymer.

13-14. (canceled)

15. The method of claim 12, wherein the cell-free extract comprises an S150 extract prepared from mid- to late-exponential growth phase cell cultures or cultures having an OD₆₀₀ or at least about 2.0, 2.5, or 3.0 at time of harvest.

16. The method of claim 12, wherein the mRNA encoding the sequence-defined polymer comprise a modified Shine-Dalgarno sequence and the engineered ribosome of the second translation system comprises an anti-Shine-Dalgarno sequence complementary to the modified Shine-Dalgarno sequence of the mRNA.

17-22. (canceled)

23. One or more polynucleotides, the one or more polynucleotides encoding the rRNA of the engineered ribosome of a) the first protein translation mechanism and/or encoding the rRNA of the engineered ribosome of b) the second protein translation mechanism of the engineered cell of claim 1.

24. The polynucleotide of claim 23, wherein the polynucleotide is a vector.

25. The polynucleotide of claim 23, wherein the polynucleotide further comprises a gene to be expressed by the engineered ribosome.

26-32. (canceled)

33. A method for preparing an engineered ribosome, the method comprising expressing the polynucleotide of claim 23 in a host cell, optionally wherein the host cell comprises an engineered cell of claim 1.

34. The method of claim 33, the method further comprising subjecting the host cell to selection and selecting a host cell comprising a mutant ribosome.

35. The method of claim 34, wherein the mutant comprises a mutation in one or more of:
a) peptidyl transferase center (PTC);
b) nascent peptide exit tunnel (NPET);
c) interaction site with elongation factors;
d) tRNA binding sites;
e) chaperone binding sites;
f) nascent chain modifying enzyme biding sites;
g) GTPase center;
h) interaction site with the translocon; and
i) interaction sites with the auxiliary proteins facilitating translation.

36. (canceled)
